(19) United States
(12) Patent Application Publication
(10) Pub. No.: US 2017/0304182 A1
(43) Pub. Date: Oct. 26, 2017

(54) CONDITIONING COMPOSITIONS

(71) Applicant: Lubrizol Advanced Materials, Inc., Cleveland, OH (US)

(72) Inventors: Murat Kadir, Brecksville, OH (US); Dongcui Li, North Royalton, OH (US); Neil Howard, Hudson, OH (US); Brian D. Figura, Cleveland, OH (US)

(21) Appl. No.: 15/507,334

(22) PCT Filed: Aug. 25, 2015
(86) PCT No.: PCT/US2015/046651

§ 371 Date: Feb. 28, 2017

Related U.S. Application Data
(60) Provisional application No. 62/042,824, filed on Aug. 28, 2014.

(51) Int. Cl. A61K 8/81; A61K 8/06; A61K 8/19; C08F 220/28; A61Q 5/12; A61K 8/02; A61K 8/60; A61K 8/49; A61K 8/41; A61K 8/34; A61K 8/86; A61K 8/89

(52) U.S. Cl. CPC A61K 8/8152; A61K 8/86; A61K 8/06; A61K 8/19; C08F 220/28; A61Q 5/12; A61K 8/89; A61K 8/0241; A61K 8/608; A61K 8/49; A61K 8/416; A61K 8/345; A61K 2800/262; A61K 2800/5422; C08F 2220/281

(57) ABSTRACT

A hair conditioning composition comprising: a) at least one cationic surfactant compound; b) at least one fatty acid ester of an ethoxylated alkyl glucoside; c) at least one nonionic, amphiphilic, emulsion polymer; d) an aqueous carrier; and optionally, e) at least one other auxiliary conditioning agent different than (a) said other conditioning agent is selected from a silicone, a hydrocarbon oil, a natural oil, an ester oil, a cationic compound, a cationic polymer, and combinations thereof.

### FIELD

Certain embodiments of the present technology relate to clear hair conditioning compositions that impart improved conditioning properties to the hair. Additionally, certain embodiments of the present technology concern a phase stable hair conditioning composition comprising a cationic surfactant, a fatty acid ester of an ethoxylated alkyl glucoside and a nonionic, amphiphilic, emulsion polymer capable of indefinitely suspending an insoluble agent for topical delivery to the hair and scalp.

### BACKGROUND

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds and aqueous carrier, are believed to provide a gel matrix which is suitable for providing a variety of conditioning benefits such as slippery feel during the application to wet hair and softness and moisturized feel on dry hair. However, the high melting point fatty compounds, low melting oils and silicone emulsions are not water soluble and result in products which are translucent or opaque. Many consumers prefer clear products for their personal care needs and associate clarity with cleanliness, freshness and environmental friendliness. In addition, components that provide clear compositions give the manufacturer wider latitude in formulating the final product.

Consumers also expect personal care products to have an aesthetically pleasing viscosity. Formulations that flow with a watery consistency are aesthetically unpopular to consumers with expectations of thick and rich products. A thicker, richer product appearance is appealing to consumers from an aesthetic and sensory perspective. While low viscosity products may be effective for their intended purpose, they are perceived to be of low quality by the consumer. Formulations that flow with a watery consistency run off when applied to the hair and scalp. For a cosmetic conditioning composition to be effective, it often must have substantively to the hair and scalp. Higher viscosity also is desired for controlled handling and dispensing of the product during use as compared to a thinner product.

Many common thickeners such as xanthan gum, CMC (carboxymethylcellulose), carrageenan, and polyacrylates (e.g., crosslinked (meth)acrylic acid polymers and copolymers) are anionic and therefore, can react with the cationic surfactants and cause precipitation of the thickener or reduce the efficacy of the cationic surfactant. Some forms of polyacrylate polymers are effective at thickening cationic systems but they can be limited by pH, require high concentrations, have high cost-in-use, and often have narrow limits of compatibility with cationic surfactants. Non-ionic thickeners such as hydroxyethylcellulose (HEC), and hydroxypropylmethylcellulose (HPMC) can provide viscosity in cationic systems, however, in the case of HEC and HPMC, very little suspension properties are imparted to the composition. Cationic thickeners such as polyquaternium 10 (cationic HEC) and cationic guar provide thickening in cationic systems but not suspension.

There is a need in industry to provide reliable suspension as well as viscosity and clarity to cationic conditioning systems. In these systems, the suspension of particles and insoluble materials is often desired and such particulates and insoluble materials include aesthetic agents (decorative beads, pearlizing agents, air bubbles, fragrance beads, etc.) or active ingredients (insoluble enzymes, insoluble silicones, insoluble vitamins, etc.), encapsulated actives (moisturizers, peptides, proteins, and vitamins).

Suspending agents are commonly employed in a variety of hair treatment compositions to improve stability against separation of the components, especially settling of suspended materials. Examples of suspending agents commonly used in hair treatment compositions include crystalline suspending agents (such as ethylene glycol distearate) and inorganic structurants (such as swelling clays). Although these materials are effective for suspending particulates and insoluble materials, they can adversely impart an undesirable cloudy appearance to the composition. Furthermore, during use of the composition they tend to get co-deposited along with the ingredients that is desired to deposit, which can lead to dulling of the hair through excessive build up and reduced performance.

Rheology modifiers have been used in aqueous hair treatment products, such as, for example, shampoos and hair conditioners, to increase their viscosity and/or to increase the yield stress (suspension stability) of the composition. While a certain rheology modifier may thicken or enhance the viscosity of a composition in which it is included, it does not necessarily have desirable yield stress properties. A desirable yield stress property is critical to achieving certain physical and aesthetic characteristics in a liquid medium, such as the indefinite suspension of particles, insoluble liquid droplets, or the stabilization of gas bubbles within a liquid medium. Particles dispersed in a liquid medium will remain suspended if the yield stress (yield value) of the medium is sufficient to overcome the effect of gravity or buoyancy on those particles. Insoluble liquid droplets can be prevented from rising and coalescing and gas bubbles can be suspended and uniformly distributed in a liquid medium using yield value as a formulating tool. A yield stress fluid is used generally to adjust or modify the rheological properties of aqueous compositions. Such properties include, without limitation, viscosity improvement, flow rate improvement, stability to viscosity change over time, and the ability to suspend particles for indefinite periods of time.

Many rheology modifiers that are employed as suspending agents operate on the principle of thickening a liquid product to a great enough viscosity to retard the phase separation of particulate and/or insoluble materials to such an extent that the product is stable over its lifetime. However, a suspending agent relying only on thickening must be incorporated in such a high percentage to provide long term suspension that an unacceptably viscous product results. An increase in viscosity alone is not sufficient to afford permanent suspension of a dispersed phase. Stokes' law provides that merely increasing viscosity will delay but not stop separation or sedimentation of particles or droplets suspended in a liquid. This assumes of course that the particles are too large to be suspended by Brownian motion. Conditioning products having too high a viscosity are not acceptable to consumers since they are hard to dispense and difficult to spread evenly on the hair and scalp, and often do not generate adequate foam. The ideal conditioner should be thick enough to appear concentrated and rich and not run out of the container or hands too easily during application, and be thin enough for easy dispensing from the container, as well as ease of application to the hair and even distribution over the scalp.

There are drawbacks associated with increasing the viscosity of a product beyond its ideal viscosity. Highly viscous products are typically difficult to apply and rinse away, especially if the shear thinning profile of the viscosity building agent is poor. High viscosities can also adversely affect packaging, dispensing, dissolution, and the sensory properties of the product.

There remains the challenge of formulating cationic surfactant based hair conditioner compositions which can effectively suspend particulate and insoluble materials, while at the same time achieving good viscosity profiles, clarity and suspension stability.

### SUMMARY

It has been discovered that aqueous, cationic surfactant containing hair conditioning compositions achieving good viscosity profiles, sensory aesthetics, clarity and suspension stability are obtained by incorporating at least one nonionic, amphiphilic polymer in combination with at least one fatty acid ester of an ethoxylated alkyl glucoside into the formulation to provide stable hair conditioning composition.

In one aspect, the disclosed technology relates to a hair conditioning composition containing in an aqueous medium:

a) at least one cationic surfactant compound;
b) at least one fatty acid ester of an ethoxylated alkyl glucoside;
c) at least one nonionic, amphiphilic, emulsion polymer;
d) aqueous carrier; and optionally
e) at least one other auxiliary conditioning agent different than (a) said other conditioning agent is selected from a silicone, a hydrocarbon oil, a natural oil, an ester oil, a cationic compound, a cationic polymer, and combinations thereof wherein the emulsion polymer is prepared from a polymerizable monomer mixture comprising at least one hydrophilic monomer and at least one hydrophobic monomer; wherein said hydrophilic monomer is selected from hydroxy(C₁-C₅)alkyl (meth)acrylates, N-vinyl amides, amino group containing monomers, or mixtures thereof; wherein said hydrophobic monomer is selected from esters of (meth)acrylic acid with alcohols containing 1 to 30 carbon atoms, vinyl esters of aliphatic carboxylic acids containing 1 to 22 carbon atoms, vinyl ethers of alcohols containing 1 to 22 carbon atoms, vinyl aromatic monomers, vinyl halides, vinylidene halides, associative monomers, semi-hydrophobic monomers, or mixtures thereof.

In one aspect, embodiments of the present technology relate to a stable hair conditioning composition containing in an aqueous medium:

a) at least one cationic surfactant compound;
b) at least one fatty acid ester of an ethoxylated alkyl glucoside;
c) at least one nonionic, amphiphilic, emulsion polymer;
d) aqueous carrier;
e) at least one silicone conditioning agent; and optionally
f) at least one auxiliary conditioning agent different than (a) said other conditioning agent is selected from a silicone, a hydrocarbon oil, a natural oil, an ester oil, a cationic compound (different from (a)), a cationic polymer, and combinations thereof; wherein the emulsion polymer is prepared from a polymerizable monomer mixture comprising at least one hydrophilic monomer and at least one hydrophobic monomer, wherein said hydrophilic monomer is selected from hydroxy(C₁-C₅)alkyl (meth)acrylates, N-vinyl amides, amino group containing monomers, or mixtures thereof; wherein said hydrophobic monomer is selected from esters of (meth)acrylic acid with alcohols containing 1 to 30 carbon atoms, vinyl esters of aliphatic carboxylic acids containing 1 to 22 carbon atoms, vinyl ethers of alcohols containing 1 to 22 carbon atoms, vinyl aromatic monomers, vinyl halides, vinylidene halides, associative monomers, semi-hydrophobic monomers, or mixtures thereof.

In another aspect, an embodiment of the disclosed technology relates to a composition and method for improving the suspension stability of a thickened aqueous hair conditioning composition comprising:

a) at least one cationic surfactant compound;
b) at least one fatty acid ester of an ethoxylated alkyl glucoside;
c) at least one nonionic, amphiphilic, emulsion polymer;
d) aqueous carrier;
e) at least one silicone conditioning agent; and optionally
f) at least one other auxiliary conditioning agent, said other conditioning agent is selected from a silicone, a hydrocarbon oil, a natural oil, an ester oil, a cationic compound (different than (a)), a cationic polymer, and combinations thereof; wherein the emulsion polymer is prepared from a polymerizable monomer mixture comprising at least one hydrophilic monomer and at least one hydrophobic monomer, wherein said hydrophilic monomer is selected from hydroxy(C₁-C₅)alkyl (meth)acrylates, N-vinyl amides, amino group containing monomers, or mixtures thereof; wherein said hydrophobic monomer is selected from esters of (meth)acrylic acid with alcohols containing 1 to 30 carbon atoms, vinyl esters of aliphatic carboxylic acids containing 1 to 22 carbon atoms, vinyl ethers of alcohols containing 1 to 22 carbon atoms, vinyl aromatic monomers, vinyl halides, vinylidene halides, associative monomers, semi-hydrophobic monomers, or mixtures thereof; and wherein the yield stress of the composition is at least 0.1 Pa with a shear thinning index of less than 0.5 at shear rates between about 0.1 and about 1 reciprocal seconds, and wherein the yield stress, elastic modulus and optical clarity of the composition are substantially independent of pH ranging from about 2 to about 14.

In one aspect of the disclosed technology, the nonionic, amphiphilic emulsion polymer (c) is prepared from a free radically polymerizable monomer composition comprising at least one hydrophilic monomer, at least one hydrophobic monomer, and at least one crosslinking monomer, wherein the hydrophilic monomer is selected from hydroxy(C₁-C₅)alkyl (meth)acrylates, N-vinyl amides, amino(C₁-C₅)alkyl (meth)acrylates, amino group containing monomers, or mixtures thereof and the hydrophobic monomer is selected from vinyl ester of an aliphatic carboxylic acid containing an acyl moiety having 2 to 22 carbon atoms, esters of (meth)acrylic acid with alcohols containing 1 to 30 carbon atoms, vinyl ethers of alcohols containing 1 to 22 carbon atoms, vinyl aromatic monomers, vinyl halides, vinylidene halides, associative monomers, semi-hydrophobic monomers, or mixtures thereof, and the crosslinking monomer is selected from at least one polyunsaturated monomer containing at least two polymerizable unsaturated moieties.

In one aspect of the disclosed technology, the nonionic, amphiphilic emulsion polymer (c) is prepared from a free radically polymerizable monomer composition comprising at least one hydroxy(C₁-C₅)alkyl (meth)acrylate, at least one ester of (meth)acrylic acid with alcohols containing 1 to 30 carbon atoms, at least one associative monomer, and at least one crosslinking monomer.

### DESCRIPTION OF EXEMPLARY EMBODIMENTS

Exemplary embodiments in accordance with the disclosed technology will be described. Various modifications, adaptations or variations of the exemplary embodiments described herein may become apparent to those skilled in the art as such are disclosed. It will be understood that all such modifications, adaptations or variations that rely upon the teachings of the disclosed technology, and through which these teachings have advanced the art, are considered to be within the scope and spirit of the presently disclosed technology.

The compositions, polymers and methods of the disclosed technology may suitably comprise, consist of, or consist essentially of the components, elements, steps, and process delineations described herein. The technology illustratively disclosed herein suitably may be practiced in the absence of any element which is not specifically disclosed herein.

Except as otherwise noted, the articles “a”, “an”, and “the” mean one or more.

The phrase “at least one” as used herein means one or more and thus includes individual components as well as mixtures or combinations of individual components.

Unless otherwise stated, all percentages, parts, and ratios expressed herein are based upon weight of the total compositions of the disclosed technology.

When referring to a specified monomer(s) that is incorporated into a polymer of the disclosed technology, it will be recognized that the monomer(s) will be incorporated into the polymer as a unit(s) derived from the specified monomer(s) (e.g., repeating unit).

As used herein, the term “amphiphilic polymer” means that the polymeric material has distinct hydrophilic and hydrophobic portions. “Hydrophilic” typically means a portion that interacts intermolecularly as well as intramolecularly with water and other polar molecules. “Hydrophobic” typically means a portion that interacts preferentially with oils, fats or other non-polar molecules rather than aqueous media.

As used herein, the term “hydrophilic monomer” means a monomer that is substantially water soluble. “Substantially water soluble” refers to a material that is soluble in distilled (or equivalent) water, at 25° C., at a concentration of about 3.5% by weight in one aspect, and soluble at about 10% by weight in another aspect (calculated on a water plus monomer weight basis).

As used herein, the term “hydrophobic monomer” means a monomer that is substantially water insoluble. “Substantially water insoluble” refers to a material that is not soluble in distilled (or equivalent) water, at 25° C., at a concentration of about 3% by weight in one aspect, and not soluble at about 2.5% by weight in another aspect (calculated on a water plus monomer weight basis).

By “nonionic” is meant that a monomer, monomer composition or a polymer polymerized from a monomer composition is devoid of ionic or ionizable moieties (“nonionizable”).

An ionizable moiety is any group that can be made ionic by neutralization with an acid or a base.

An ionic or an ionized moiety is any moiety that has been neutralized by an acid or a base.

By “substantially nonionic” is meant that the monomer, monomer composition or polymer polymerized from a monomer composition contains less than 5 wt. % in one aspect, less than 3 wt. % in another aspect, less than 1 wt. % in a further aspect, less than 0.5 wt. % in a still further aspect, less than 0.1 wt. % in an additional aspect, and less than 0.05 wt. % in a further aspect, of an ionizable and/or an ionized moiety.

The prefix “(meth)acryl” includes “acryl” as well as “methacryl”. For example, the term (meth)acrylic includes both acrylic and methacrylic, and the term (meth)acrylate includes acrylate as well as methacrylate. By way of further example, the term “(meth)acrylamide” includes both acrylamide and methacrylamide.

Here, as well as elsewhere in the specification and claims, individual numerical values (including carbon atom numerical values), or limits, can be combined to form additional non-disclosed and/or non-stated ranges.

While overlapping weight ranges for the various components and ingredients that can be contained in the compositions of the disclosed technology have been expressed for selected embodiments and aspects of the technology, it should be readily apparent that the specific amount of each component in the disclosed compositions will be selected from its disclosed range such that the amount of each component is adjusted such that the sum of all components in the composition will total 100 weight percent. The amounts employed will vary with the purpose and character of the desired product and can be readily determined by one skilled in the art.

The headings provided herein serve to illustrate, but not to limit the disclosed technology in any way or manner.

### Cationic Surfactant Compound

In one aspect, the at least one cationic surfactant component (a) of the hair conditioning composition of the present technology is selected from a monoalkyl quaternary compound. Monoalkyl quaternary compounds can be represented by the formula:

(R′″)(R″)(R′)(R)N⁺A⁻

wherein R′, R″, and R′″ are independently selected from a C1 to C3 alkyl group; R is selected from an alkyl group of from 12 to 22 carbon atoms; and A is a salt-forming anion such as, for example, those selected from halogen, (e.g. chloride, bromide), acetate, citrate, lactate, glycolate, phosphate, nitrate, sulphate, and alkylsulfate (e.g, methosulfate).

In one aspect, the at least one cationic surfactant compound is selected from a monoalkyl quaternary ammonium compound where R represents a C₁₄-C₂₂ alkyl radical, R′, R″, R′″ independently represent a C₁-C₃ alkyl (e.g., methyl, ethyl, and propyl); and A is a salt-forming anion such as described above.

In one aspect, the at least one cationic compound component (a) is selected from a monoalkyl quaternary compound represented by the formula:

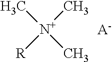

where R represents a C₁₂-C₂₂ alkyl radical, and A is an anion selected from chloride, bromide and methosulfate.

Exemplary cationic surfactant compounds include, but are not limited to, dodecyltrimethylammonium chloride, cetyltrimethylammonium chloride (cetrimonium chloride), palmityltrimethylammonium chloride, stearyltrimethylammonium chloride, tallowtrimethylammonium chloride, soytrimonium chloride, cocotrimethylammonium chloride, behenyltrimethylammonium chloride, eicosyltrimethylammonium chloride, and salts of these compounds where the chloride anion is replaced by another halide, (e.g., bromide), acetate, citrate, lactate, glycolate, phosphate nitrate, sulfate, or alkylsulfate (e.g., methosulfate).

In one aspect, a useful cationic conditioning surfactant is cetyltrimethylammonium chloride (CTAC), available commercially, for example, from the Stepan Company under the trade name Ammonyx® Cetac-30 or from Clariant Corporation under the trade name Genamin® CTAC 25.

The amount of cationic surfactant (active material basis) utilized in the compositions of the present technology range from about 1 to about 10 wt. % in one aspect, from about 2 to about 6 wt. % in another aspect, and from about 3 to about 5 wt. % in still another aspect, based on the weight of the total composition.

### Fatty Acid Ester of an Ethoxylated Alkyl Glucoside (Glucoside Ester)

The at least one fatty acid ester of the ethoxylated alkyl glucoside component (b) is selected from a compound represented by the formula:

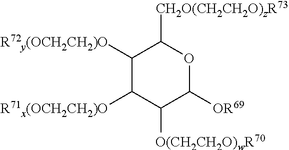

wherein R⁶⁹ represents C₁ to C₅ alkyl, R⁷⁰, R⁷¹, R⁷² and R⁷³ independently represent hydrogen or an acyl substituent represented by —C(O)R⁷⁴, where R⁷⁴ is selected from C₅ to C₂₁ alkyl, C₅ to C₂₁ alkenyl, and combinations thereof, subject to the proviso that at least one of R⁷⁰ to R⁷⁴ must be selected from said acyl substituent, and wherein the sum of w+x+y+z ranges from about 50 to about 400 in one aspect, from about 80 to about 180 in another aspect, from about 100 to about 160 in a further aspect, and from about 110 to about 130 in a still further aspect.

In one aspect, the acyl substituent represented by the radical —C(O)R⁷⁴ is derived from a saturated or unsaturated carboxylic or fatty acid obtained from natural or synthetic sources. These acids can be linear or branched. Suitable acids are selected from, but are not limited to caproic acid, enanthic acid, caprylic acid, pelargonic acid, capric acid, undecanoic acid lauric acid, myristic acid, palmitic acid, stearic acid, isosteric acid, oleic acid, linoleic acid, ricinoleic acid, and behenic acid which are typically obtained by hydrolyzing vegetable oils and animal oils such as coconuts oils, palm oil, tallow, linseed oil and soybean oil. Examples of synthetic fatty acids, include, but are not limited to, linear or branched fatty acids prepared by oxidizing olefin polymers. It is also possible to use fatty acids derived from microorganisms such as, for example, γ-linolenic acid. Further, as the lower alkyl ester of the fatty acid, alkyl esters having 1 to 8 carbon atoms such as methyl, ethyl or propyl ester of the fatty acid described above can be used.

In one aspect of the present technology, the average substitution level of the acyl substituent is about 3 in one aspect, from about 2.5 to about 4 in another aspect, from about 2.5 to about 3.9 in a further aspect, and from about 2.8 to 3.6 in a still further aspect. Here, as well as elsewhere in the specification and claims, individual numerical values, or limits, can be combined to form additional non-disclosed and/or non-stated ranges. Details concerning the derivatization of the glucoside ester component of the hair conditioning compositions of the present technology are known to those skilled in the art such as disclosed in U.S. Pat. No. 6,573,375 and International Publication No. 2012/033783, the disclosures of which are herein incorporated by reference.

In accordance with one aspect of the present technology, the glucoside ester comprises a mixture of compounds substituted with varying amounts of the acyl substituent depending upon the available hydroxyl groups on the polyol starting material. At least 5 percent of the polyol compounds in the composition have about three moles of the acyl substituent per mole of polyol. For example, in the case of an ethoxylated and esterified methyl glucoside, at least about 5 percent of the compounds are substituted with about three moles of the acyl substituent per mole of the methyl glucoside. Typically, at least about 25 percent, or at least about 50 percent, or even at least about 75 percent of the ethoxylated and esterified polyol derivatives in the composition have about three moles of the acyl substituent per mole of polyol starting material. Here, as well as elsewhere in the specification and claims, individual numerical values, or limits, can be combined to form additional non-disclosed and/or non-stated ranges. Typically, the balance of the composition comprises ethoxylated and esterified polyol derivatives having one, two or four moles of the acyl substituent per mole of polyol starting material. In one aspect, less than about 75 percent, or less than about 50 percent, or even less than about 25 percent of the polyols in the composition comprise one, two or four moles of the acyl substituent per mole of polyol. Here, as well as elsewhere in the specification and claims, individual numerical values, or limits, can be combined to form additional non-disclosed and/or non-stated ranges.

In accordance with another aspect of the present technology, the glucoside ester component comprises a mixture of compounds substituted with varying amounts of the acyl substituent depending upon the available hydroxyl groups on the polyol starting material. At least 5 percent of the polyol compounds in the composition have about two moles of the acyl substituent per mole of polyol. For example, in the case of an ethoxylated, esterified methyl glucoside, at least about 5 percent of the compounds are substituted with about two moles of the acyl substituent per mole of the methyl glucoside. Typically, at least about 25 percent, or at least about 50 percent, or even at least about 75 percent ethoxylated and esterified polyol derivatives in the composition have about two moles of the acyl substituent per mole of polyol starting material. Here, as well as elsewhere in the specification and claims, individual numerical values, or limits, can be combined to form additional non-disclosed and/or non-stated ranges. Typically, the balance of the composition comprises polyol derivatives having one, two or four moles of the acyl substituent per mole of polyol. In one embodiment, less than about 75 percent, or less than about 50 percent, or even less than about 25 percent of the polyols in the composition comprise one, two or four moles of the lipophilic substituent per mole of polyol. Here, as well as elsewhere in the specification and claims, individual numerical values, or limits, can be combined to form additional non-disclosed and/or non-stated ranges.

In one aspect, the glucoside ester is in the form of a solid provided in a granulated, flake or powdered form. In one aspect the solid glucoside ester is a methyl glucose derivative which is esterified with about 2 moles of oleic acid and ethoxylated with about 110 to about 130 moles of ethylene oxide. In one aspect, the solid glucoside ester is commercially available under the trade name Glucamate™ DOE-120 (CTFA/INCI Designation: PEG-120 Methyl Glucose Dioleate) available from Lubrizol Advanced Materials, Inc.

In another aspect, the glucoside ester compounds of the present technology are dissolved in a suitable solvent to provide a liquid composition suitable for use in liquid compositions. Any suitable solvent, or solvents, capable of dissolving the glucoside ester is suitable for use in accordance with the present technology. In one aspect, the solvent is water with or without additional water miscible solvents. Suitable solvents include, but are not limited to, alkylene glycols having about 2 to about 5 carbon atoms per molecule (e.g., propylene glycol, ethylene glycol, butylene glycol), propanediol (e.g., 1,2-propanediol, 1,3-propanediol) and butanediol. Dialkylene glycols (e.g., diethylene and dipropylene glycols) also can be utilized as suitable solvents. Other solvents, such as for example, polyalkylene glycols such as Carbowax™ PEG and Ucon™ fluids available from Dow Chemical can be utilized. Mixtures of two or more of the above mentioned solvents can be utilized.

In one aspect of the disclosed technology, the glucoside ester component is provided in a liquid composition containing a methyl glucose derivative which is esterified with about 3 moles of oleic acid and ethoxylated with about 110 to about 130 moles of ethylene oxide. In one aspect, the liquid composition comprises from about 10 to 30 wt. % water, from about 30 to 50 wt. % propylene glycol and from about 30 to 50 wt. % of the glucoside ester. In one aspect the liquid composition comprises about 20 wt. % water, about 40 wt. % propylene glycol and about 40 wt. % of the glucoside ester derivative. In one aspect, the liquid glucoside ester is commercially available under the trade name Glucamate™ LT (CTFA/INCI Designation: PEG-120 Methyl Glucose Trioleate (and) Propylene Glycol (and) Water) available from Lubrizol Advanced Materials, Inc.

In one aspect of the disclosed technology, the glucoside ester component is provided in a liquid composition containing a methyl glucose derivative which is esterified with about 3 moles of oleic acid and ethoxylated with about 110 to about 130 moles of ethylene oxide. In one aspect, the liquid composition comprises from about 15 to about 25 wt. % water, from about 8 to about 22 wt. % of an alkylene glycol such as propanediol (e.g., 1,2-propanediol, 1,3-propanediol) and from about 55 to about 75 wt. % of the glucoside ester. In one aspect, the liquid glucoside ester is commercially available under the trade name Glucamate™ VLT (CTFA/INCI Designation: PEG-120 Methyl Glucose Trioleate (and) Propanediol) available from Lubrizol Advanced Materials, Inc.

The amount of glucoside ester (active material) utilized in the compositions of the present technology range from about 0.5 to about 10 wt. % in one aspect, from about 1 to about 6 wt. % in another aspect, and from about 1.5 to about 3 wt. % in still another aspect, based on the weight of the total composition.

### Ampholytic Polymer

The nonionic, amphiphilic emulsion polymer component (c) useful in the compositions of the disclosed technology are polymerized from monomer components that contain free radical polymerizable unsaturation. In one embodiment, the nonionic, amphiphilic polymers useful in the practice of the disclosed technology are polymerized from a monomer composition comprising at least one nonionic, hydrophilic unsaturated monomer, and at least one unsaturated hydrophobic monomer. In another embodiment, the nonionic, amphiphilic polymers useful in the practice of the disclosed technology are crosslinked. The crosslinked polymers are prepared from a monomer composition comprising at least one nonionic, hydrophilic unsaturated monomer, at least one unsaturated hydrophobic monomer, and at least one polyunsaturated crosslinking monomer.

In one embodiment, the copolymers can be prepared from a monomer composition typically having a hydrophilic monomer to hydrophobic monomer ratio of from about 5:95 wt. % to about 95:5 wt. % in one aspect, from about 15:85 wt. % to about 85:15 wt. % in another aspect, and from about 30:70 wt. % to about 70:30 wt. % in a further aspect, based on the total weight of the hydrophilic and hydrophobic monomers present. The hydrophilic monomer component can be selected from a single hydrophilic monomer or a mixture of hydrophilic monomers, and the hydrophobic monomer component can be selected from a single hydrophobic monomer or a mixture of hydrophobic monomers.

### Hydrophilic Monomer

The hydrophilic monomers suitable for the preparation of the crosslinked, nonionic, amphiphilic polymer compositions of the disclosed technology are selected from but are not limited to hydroxy(C₁-C₅)alkyl (meth)acrylates; open chain and cyclic N-vinylamides (N-vinyllactams containing 4 to 9 atoms in the lactam ring moiety, wherein the ring carbon atoms optionally can be substituted by one or more lower alkyl groups such as methyl, ethyl or propyl); amino group containing vinyl monomers selected from (meth)acrylamide, N—(C₁-C₅)alkyl(meth)acrylamides, N,N-di(C₁-C₅)alkyl(meth)acrylamides, N—(C₁-C₅)alkylamino(C₁-C₅)alkyl(meth)acrylamides and N,N-di(C₁-C₅)alkylamino(C₁-C₅)alkyl(meth)acrylamides, wherein the alkyl moieties on the disubstituted amino groups can be the same or different, and wherein the alkyl moieties on the monosubstituted and disubstituted amino groups can be optionally substituted with a hydroxyl group; other monomers include vinyl alcohol; vinyl imidazole; and (meth)acrylonitrile. Mixtures of the foregoing monomers also can be utilized.

The hydroxy(C₁-C₅)alkyl (meth)acrylates can be structurally represented by the following formula:

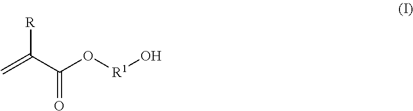

wherein R is hydrogen or methyl and R¹ is an divalent alkylene moiety containing 1 to 5 carbon atoms, wherein the alkylene moiety optionally can be substituted by one or more methyl groups. Representative monomers include 2-hydroxyethyl(meth)acrylate, 3-hydroxypropyl(meth)acrylate, 4-hydroxybutyl(meth)acrylate, and mixtures thereof.

Representative open chain N-vinylamides include N-vinylformamide, N-methyl-N-vinylformamide, N-(hydroxymethyl)-N-vinylformamide, N-vinylacetamide, N-vinylmethylacetamide, N-(hydroxymethyl)-N-vinylacetamide, and mixtures thereof.

Representative cyclic N-vinylamides (also known as N-vinyllactams) include N-vinyl-2-pyrrolidinone, N-(1-methyl vinyl) pyrrolidinone, N-vinyl-2-piperidone, N-vinyl-2-caprolactam, N-vinyl-5-methyl pyrrolidinone, N-vinyl-3,3-dimethyl pyrrolidinone, N-vinyl-5-ethyl pyrrolidinone and N-vinyl-6-methyl piperidone, and mixtures thereof. Additionally, monomers containing a pendant N-vinyl lactam moiety can also be employed, e.g., N-vinyl-2-ethyl-2-pyrrolidone (meth)acrylate.

The amino group containing vinyl monomers include (meth)acrylamide, diacetone acrylamide and monomers that are structurally represented by the following formulas:

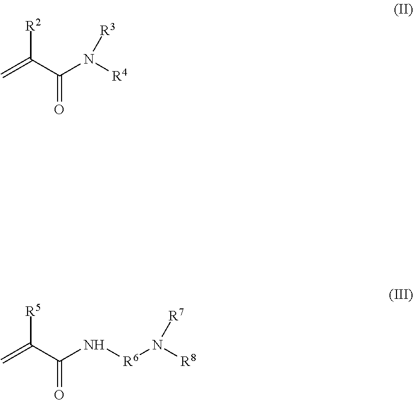

Formula (II) represents N—(C₁-C₅)alkyl(meth)acrylamide or N,N-di(C₁-C₅)alkyl(meth)acrylamide wherein R² is hydrogen or methyl, R³ independently is selected from hydrogen, C₁ to C₅ alkyl and C₁ to C₅ hydroxyalkyl, and R⁴ independently is selected from is C₁ to C₅ alkyl or C₁ to C₅ hydroxyalkyl.

Formula (III) represents N—(C₁-C₅)alkylamino(C₁-C₅)alkyl(meth)acrylamide or N,N-di(C₁-C₅)alkylamino(C₁-C₅)alkyl(meth)acrylamide wherein R⁵ is hydrogen or methyl, R⁶ is C₁ to C₅ alkylene, R⁷ independently is selected from hydrogen or C₁ to C₅ alkyl, and R⁸ independently is selected from C₁ to C₅ alkyl.

Representative N-alkyl(meth)acrylamides include but are not limited to N-methyl(meth)acrylamide, N-ethyl(meth)acrylamide, N-propyl(meth)acrylamide, N-isopropyl(meth)acrylamide, N-tert-butyl(meth)acrylamide, N-(2-hydroxyethyl)(meth)acrylamide, N-(3-hydroxypropyl)(meth)acrylamide, and mixtures thereof.

Representative N,N-dialkyl(meth)acrylamides include but are not limited to N,N-dimethyl(meth)acrylamide, N,N-diethyl(meth)acrylamide, N,N-(di-2-hydroxyethyl)(meth)acrylamide, N,N-(di-3-hydroxypropyl)(meth)acrylamide, N-methyl, N-ethyl(meth)acrylamide, and mixtures thereof.

Representative N,N-dialkylaminoalkyl(meth)acrylamides include but are not limited to N,N-dimethylaminoethyl(meth)acrylamide, N,N-diethylaminoethyl(meth)acrylamide, N,N-dimethylaminopropyl(meth)acrylamide, and mixtures thereof.

### Hydrophobic Monomer

Hydrophobic monomers suitable for the preparation of the crosslinked, nonionic, amphiphilic polymer compositions of the disclosed technology are selected from but are not limited to one or more of alkyl esters of (meth)acrylic acid having an alkyl group containing 1 to 30 carbon atoms; vinyl esters of aliphatic carboxylic acids containing 1 to 22 carbon atoms; vinyl ethers of alcohols containing 1 to 22 carbon atoms; vinyl aromatics containing 8 to 20 carbon atoms; vinyl halides; vinylidene halides; linear or branched alpha-monoolefins containing 2 to 8 carbon atoms; an associative monomer having a hydrophobic end group containing 8 to 30 carbon atoms, and mixtures thereof.

### Semi-Hydrophobic Monomer

Optionally, at least one alkoxylated semi-hydrophobic monomer can be used in the preparation of the amphiphilic polymers of the disclosed technology. A semi-hydrophobic monomer is similar in structure to an associative monomer, but has a substantially non-hydrophobic end group selected from hydroxyl or a moiety containing 1 to 4 carbon atoms.

In one aspect, of the disclosed technology, alkyl esters of (meth)acrylic acid having an alkyl group containing 1 to 22 carbon atoms can be represented by the following formula:

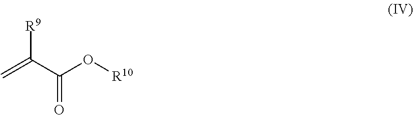

wherein R⁹ is hydrogen or methyl and R¹⁰ is C₁ to C₂₂ alkyl

Representative monomers under formula (IV) include but are not limited to methyl (meth)acrylate, ethyl (meth)acrylate, butyl (meth)acrylate, sec-butyl (meth)acrylate, iso-butyl (meth)acrylate, hexyl (meth)acrylate), heptyl (meth)acrylate, octyl (meth)acrylate, 2-ethylhexyl (meth)acrylate, decyl (meth)acrylate, isodecyl (meth)acrylate, lauryl (meth)acrylate, tetradecyl (meth)acrylate, hexadecyl (meth)acrylate, stearyl (meth)acrylate, behenyl (meth)acrylate, and mixtures thereof.

Vinyl esters of aliphatic carboxylic acids containing 1 to 22 carbon atoms can be represented by the following formula:

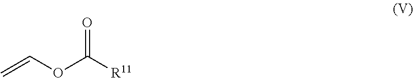

wherein R¹¹ is a C₁ to C₂₂ aliphatic group which can be an alkyl or alkenyl. Representative monomers under formula (V) include but are not limited to vinyl acetate, vinyl propionate, vinyl butyrate, vinyl isobutyrate, vinyl valerate, vinyl hexanoate, vinyl 2-methylhexanoate, vinyl 2-ethylhexanoate, vinyl iso-octanoate, vinyl nonanoate, vinyl neodecanoate, vinyl decanoate, vinyl versatate, vinyl laurate, vinyl palmitate, vinyl stearate, and mixtures thereof.

In one aspect, the vinyl ethers of alcohols containing 1 to 22 carbon atoms can be represented by the following formula:

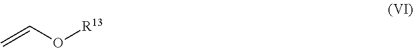

wherein R¹³ is a C₁ to C₂₂ alkyl. Representative monomers of formula (VI) include methyl vinyl ether, ethyl vinyl ether, butyl vinyl ether, isobutyl vinyl ether, 2-ethylhexyl vinyl ether, decyl vinyl ether, lauryl vinyl ether, stearyl vinyl ether, behenyl vinyl ether, and mixtures thereof.

Representative vinyl aromatic monomers include but are not limited to styrene, alpha-methylstyrene, 3-methyl styrene, 4-methyl styrene, 4-propyl styrene, 4-tert-butyl styrene, 4-n-butyl styrene, 4-n-decyl styrene, vinyl naphthalene, and mixtures thereof.

Representative vinyl and vinylidene halides include but are not limited to vinyl chloride and vinylidene chloride, and mixtures thereof.

Representative alpha-olefins include but are not limited to ethylene, propylene, 1-butene, iso-butylene, 1-hexene, and mixtures thereof.

The alkoxylated associative monomer of the disclosed technology has an ethylenically unsaturated end group portion (i) for addition polymerization with the other monomers of the disclosed technology; a polyoxyalkylene mid-section portion (ii) for imparting selective hydrophilic and/or hydrophobic properties to the product polymer, and a hydrophobic end group portion (iii) for providing selective hydrophobic properties to the polymer.

The portion (i) supplying the ethylenically unsaturated end group can be a residue derived from an α,β-ethylenically unsaturated monocarboxylic acid. Alternatively, portion (i) of the associative monomer can be a residue derived from an allyl ether or vinyl ether; a nonionic vinyl-substituted urethane monomer, such as disclosed in U.S. Reissue Pat. No. 33,156 or U.S. Pat. No. 5,294,692; or a vinyl-substituted urea reaction product, such as disclosed in U.S. Pat. No. 5,011,978; the relevant disclosures of each are incorporated herein by reference.

The mid-section portion (ii) is a polyoxyalkylene segment of about 2 to about 150 in one aspect, from about 10 to about 120 in another aspect, and from about 15 to about 60 in a further aspect of repeating C₂-C₄ alkylene oxide units. The mid-section portion (ii) includes polyoxyethylene, polyoxypropylene, and polyoxybutylene segments, and combinations thereof comprising from about 2 to about 150 in one aspect, from about 5 to about 120 in another aspect, from about 10 to about 60 in a further aspect, and from about 15 to about 30 in a still further aspect of ethylene, propylene and/or butylene oxide units, arranged in random or block sequences of ethylene oxide, propylene oxide and/or butylene oxide units.

The hydrophobic end group portion (iii) of the associative monomer is a hydrocarbon moiety belonging to one of the following hydrocarbon classes: a C₈-C₃₀ linear alkyl, a C₈-C₃₀ branched alkyl, a C₂-C₃₀ alkyl-substituted phenyl, aryl-substituted C₂-C₃₀ alkyl groups, a C₇-C₃₀ saturated or unsaturated carbocyclic alkyl group. The saturated or unsaturated carbocyclic moiety can be a C₁-C₅ alkyl substituted or unsubstituted monocyclic or bicyclic moiety. In one aspect the bicyclic moiety is selected from bicycloheptyl or bicycloheptenyl. In another aspect the bicycloheptenyl moiety is disubstituted with the alkyl substituent(s). In a further aspect the bicycloheptenyl moiety is disubstituted with methyl on the same carbon atom.

Non-limiting examples of suitable hydrophobic end group portions (iii) of the associative monomers are linear or branched alkyl groups having about 8 to about 30 carbon atoms, such as capryl (C₈), iso-octyl (branched C₈), decyl (C₁₀), lauryl (C₁₂), myristyl (C₁₄), cetyl (C₁₆), cetearyl (C₁₆-C₁₈), stearyl (C₁₈), isostearyl (branched C₁₈), arachidyl (C₂₀), behenyl (C₂₂), lignoceryl (C₂₄), cerotyl (C₂₆), montanyl (C₂₈), melissyl (C₃₀), and the like.

Examples of linear and branched alkyl groups having about 8 to about 30 carbon atoms that are derived from a natural source include, without being limited thereto, alkyl groups derived from hydrogenated peanut oil, soybean oil and canola oil (all predominately C₁₈), hydrogenated tallow oil (C₁₆-C₁₈), and the like; and hydrogenated C₁₀-C₃₀ terpenols, such as hydrogenated geraniol (branched C₁₀), hydrogenated farnesol (branched C₁₅), hydrogenated phytol (branched C₂₀), and the like.

Non-limiting examples of suitable C₂-C₃₀ alkyl-substituted phenyl groups include octylphenyl, nonylphenyl, decylphenyl, dodecylphenyl, hexadecylphenyl, octadecylphenyl, isooctylphenyl, sec-butylphenyl, and the like.

Exemplary aryl-substituted C₂-C₄₀ alkyl groups include, without limitation, styryl (e.g., 2-phenylethyl), distyryl (e.g., 2,4-diphenylbutyl), tristyryl (e.g., 2,4,6-triphenylhexyl), 4-phenylbutyl, 2-methyl-2-phenylethyl, tristyrylphenolyl, and the like.

Suitable C₇-C₃₀ carbocyclic groups include, without limitation, groups derived from sterols from animal sources, such as cholesterol, lanosterol, 7-dehydrocholesterol, and the like; from vegetable sources, such as phytosterol, stigmasterol, campesterol, and the like; and from yeast sources, such as ergosterol, mycosterol, and the like. Other carbocyclic alkyl hydrophobic end groups useful in the disclosed technology include, without limitation, cyclooctyl, cyclododecyl, adamantyl, decahydronaphthyl, and groups derived from natural carbocyclic materials, such as pinene, hydrogenated retinol, camphor, isobornyl alcohol, norbornyl alcohol, nopol and the like.

Useful alkoxylated associative monomers can be prepared by any method known in the art. See, for example, U.S. Pat. No. 4,421,902 to Chang et al.; U.S. Pat. No. 4,384,096 to Sonnabend; U.S. Pat. No. 4,514,552 to Shay et al.; U.S. Pat. No. 4,600,761 to Ruffner et al.; U.S. Pat. No. 4,616,074 to Ruffner; U.S. Pat. No. 5,294,692 to Barron et al.; U.S. Pat. No. 5,292,843 to Jenkins et al.; U.S. Pat. No. 5,770,760 to Robinson; U.S. Pat. No. 5,412,142 to Wilkerson, III et al.; and U.S. Pat. No. 7,772,421, to Yang et al., the pertinent disclosures of which are incorporated herein by reference.

In one aspect, exemplary alkoxylated associative monomers include those represented by formulas (VII) and (VIIA) as follows:

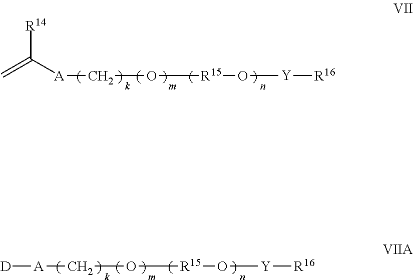

wherein R¹⁴ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; D represents a vinyl or an allyl moiety; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or C₄H₈, and combinations thereof; and n is an integer in the range of about 2 to about 150 in one aspect, from about 10 to about 120 in another aspect, and from about 15 to about 60 in a further aspect; Y is —R¹⁵O—, —R¹⁵NH—, —C(O)—, —C(O)NH—, —R¹⁵NHC(O)NH—, —C(O)NHC(O)—, or a divalent alkylene radical containing 1 to 5 carbon atoms, e.g., methylene, ethylene, propylene, butylene, pentylene; R¹⁶ is a substituted or unsubstituted alkyl selected from a C₈-C₃₀ linear alkyl, a C₈-C₃₀ branched alkyl, a C₇-C₃₀ carbocyclic, a C₂-C₃₀ alkyl-substituted phenyl, an aralkyl substituted phenyl, and an aryl-substituted C₂-C₃₀ alkyl; wherein the R¹⁶ alkyl group, aryl group, phenyl group, or carbocyclic group optionally comprises one or more substituents selected from the group consisting of a methyl group, a hydroxyl group, an alkoxyl group, benzyl group phenylethyl group, and a halogen group. In one aspect, Y is ethylene and R¹⁶ is

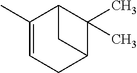

In one aspect, the hydrophobically modified alkoxylated associative monomer is an alkoxylated (meth)acrylate having a hydrophobic group containing 8 to 30 carbon atoms represented by the following Formula VIIB as follows:

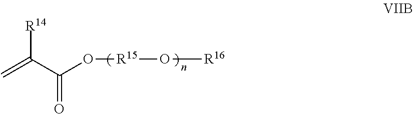

wherein R¹⁴ is hydrogen or methyl; R¹⁵ is a divalent alkylene moiety independently selected from C₂H₄, C₃H₆, and C₄H₈, and n represents an integer ranging from about 2 to about 150 in one aspect, from about 5 to about 120 in another aspect, from about 10 to about 60 in a further aspect, and from about 15 to about 30 in a still further aspect, (R¹⁵—O) can be arranged in a random or a block configuration; R¹⁶ is a substituted or unsubstituted alkyl selected from a C₈-C₃₀ linear alkyl, a C₈-C₃₀ branched alkyl, an alkyl substituted and unsubstituted C₇-C₃₀ carbocyclic alkyl, a C₂-C₃₀ alkyl-substituted phenyl, and an aryl-substituted C₂-C₃₀ alkyl.

Representative monomers under Formula VIIB include lauryl polyethoxylated (meth)acrylate (LEM), cetyl polyethoxylated (meth)acrylate (CEM), cetearyl polyethoxylated (meth)acrylate (CSEM), stearyl polyethoxylated (meth)acrylate, arachidyl polyethoxylated (meth)acrylate, behenyl polyethoxylated (meth)acrylate (BEM), cerotyl polyethoxylated (meth)acrylate, montanyl polyethoxylated (meth)acrylate, melissyl polyethoxylated (meth)acrylate, phenyl polyethoxylated (meth)acrylate, nonylphenyl polyethoxylated (meth)acrylate, ω-tristyrylphenyl polyoxyethylene (meth)acrylate, where the polyethoxylated portion of the monomer contains about 2 to about 150 ethylene oxide units in one aspect, from about 5 to about 120 in another aspect, from about 10 to about 60 in a further aspect and from about 15 to about 30 in a still further aspect; octyloxy polyethyleneglycol (8) polypropyleneglycol (6) (meth)acrylate, phenoxy polyethylene glycol (6) polypropylene glycol (6) (meth)acrylate, and nonylphenoxy polyethylene glycol polypropylene glycol (meth)acrylate.

The alkoxylated semi-hydrophobic monomers of the disclosed technology are structurally similar to the associative monomer described above, but have a substantially non-hydrophobic end group portion. The alkoxylated semi-hydrophobic monomer has an ethylenically unsaturated end group portion (i) for addition polymerization with the other monomers of the disclosed technology; a polyoxyalkylene mid-section portion (ii) for imparting selective hydrophilic and/or hydrophobic properties to the product polymer and a semi-hydrophobic end group portion (iii). The unsaturated end group portion (i) supplying the vinyl or other ethylenically unsaturated end group for addition polymerization is preferably derived from an α,β-ethylenically unsaturated mono carboxylic acid. Alternatively, the end group portion (i) can be derived from an allyl ether residue, a vinyl ether residue or a residue of a nonionic urethane monomer.

The polyoxyalkylene mid-section (ii) specifically comprises a polyoxyalkylene segment, which is substantially similar to the polyoxyalkylene portion of the associative monomers described above. In one aspect, the polyoxyalkylene portions (ii) include polyoxyethylene, polyoxypropylene, and/or polyoxybutylene units comprising from about 2 to about 150 in one aspect, from about 5 to about 120 in another aspect, from about 10 to about 60, and from about 15 to about 30 in a still further aspect in a further aspect of ethylene oxide, propylene oxide, and/or butylene oxide units, arranged in random or blocky sequences.

In one aspect, the alkoxylated semi-hydrophobic monomer can be represented by the following formulas:

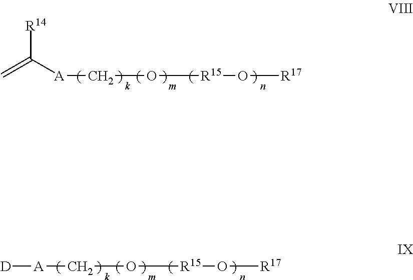

wherein R¹⁴ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or C₄H₈, and combinations thereof; and n is an integer in the range of about 2 to about 150 in one aspect, from about 5 to about 120 in another aspect, and from about 10 to about 60, and from about 15 to about 30 in a still further aspect in a further aspect; R¹⁷ is selected from hydrogen and a linear or branched C₁-C₄ alkyl group (e.g., methyl, ethyl, propyl, iso-propyl, butyl, iso-butyl, and tert-butyl); and D represents a vinyl or an allyl moiety.

In one aspect, the alkoxylated semi-hydrophobic monomer under formula VIII can be represented by the following formulas:

CH₂═C(R¹⁴)C(O)O—(C₂H₄O)ₐ(C₃H₆O)_{b}—H VIIIA

CH₂═C(R¹⁴)C(O)O—(C₂H₄O)ₐ(C₃H₆O)_{b}—CH₃ VIIIB

wherein R¹⁴ is hydrogen or methyl, and “a” is an integer ranging from 0 or 2 to about 120 in one aspect, from about 5 to about 45 in another aspect, and from about 10 to about 0.25 in a further aspect, and “b” is an integer ranging from about 0 or 2 to about 120 in one aspect, from about 5 to about 45 in another aspect, and from about 10 to about 25 in a further aspect, subject to the proviso that “a” and “b” cannot be 0 at the same time.

Examples of alkoxylated semi-hydrophobic monomers under formula VIIIA include polyethyleneglycol methacrylate available under the product names Blemmer® PE-90 (R¹⁴=methyl, a=2, b=0), PE-200 (R¹⁴=methyl, a=4.5, b=0), and PE-350 (R¹⁴=methyl a=8, b=0,); polypropylene glycol methacrylate available under the product names Blemmer® PP-1000 (R¹⁴=methyl, b=4-6, a=0), PP-500 (R¹⁴=methyl, a=0, b=9), PP-800 (R¹⁴=methyl, a=0, b=13); polyethyleneglycol polypropylene glycol methacrylate available under the product names Blemmer® 50PEP-300 (R¹⁴=methyl, a=3.5, b=2.5), 70PEP-350B (R¹⁴=methyl, a=5, b=2); polyethyleneglycol acrylate available under the product names Blemmer® AE-90 (R¹⁴=hydrogen, a=2, b=0), AE-200 (R¹⁴=hydrogen, a=2, b=4.5), AE-400 (R¹⁴=hydrogen, a=10, b=0); polypropyleneglycol acrylate available under the product names Blemmer® AP-150 (R¹⁴=hydrogen, a=0, b=3), AP-400 (R¹⁴=hydrogen, a=0, b=6), AP-550 (R¹⁴=hydrogen, a=0, b=9). Blemmer® is a trademark of NOF Corporation, Tokyo, Japan.

Examples of alkoxylated semi-hydrophobic monomers under formula VIIB include methoxypolyethyleneglycol methacrylate available under the product names Visiomer® MPEG 750 MA W (R¹⁴=methyl, a=17, b=0), MPEG 1005 MA W (R¹⁴=methyl, a=22, b=0), MPEG 2005 MA W (R¹⁴=methyl, a=45, b=0), and MPEG 5005 MA W (R¹⁴=methyl, a=113, b=0) from Evonik Röhm GmbH, Darmstadt, Germany); Bisomer® MPEG 350 MA (R¹⁴=methyl, a=8, b=0), and MPEG 550 MA (R¹⁴=methyl, a=12, b=0) from GEO Specialty Chemicals, Ambler PA; Blemmer® PME-100 (R¹⁴=methyl, a=2, b=0), PME-200 (R¹⁴=methyl, a=4, b=0), PME-400 (R¹⁴=methyl, a=9, b=0), PME-1000 (R¹⁴=methyl, a=23, b=0), PME-4000 (R¹⁴=methyl, a=90, b=0).

In one aspect, the alkoxylated semi-hydrophobic monomer set forth in formula IX can be represented by the following formulas:

CH₂═CH—O—(CH₂)_{d}—O—(C₃H₆O)ₑ—(C₂H₄O)_{f}—H IXA

CH₂═CH—CH₂—O—(C₃H₆O)_{g}—(C₂H₄O)ₕ—H IXB

wherein d is an integer of 2, 3, or 4; e is an integer in the range of from about 1 to about 10 in one aspect, from about 2 to about 8 in another aspect, and from about 3 to about 7 in a further aspect; f is an integer in the range of from about 5 to about 50 in one aspect, from about 8 to about 40 in another aspect, and from about 10 to about 30 in a further aspect; g is an integer in the range of from 1 to about 10 in one aspect, from about 2 to about 8 in another aspect, and from about 3 to about 7 in a further aspect; and h is an integer in the range of from about 5 to about 50 in one aspect, and from about 8 to about 40 in another aspect; e, f, g, and h can be 0 subject to the proviso that e and f cannot be 0 at the same time, and g and h cannot be 0 at the same time.

Monomers under formulas IXA and IXB are commercially available under the trade names Emulsogen® R109, R208, R307, RAL109, RAL208, and RAL307 sold by Clariant Corporation; BX-AA-E5P5 sold by Bimax, Inc.; and combinations thereof. EMULSOGEN7 R109 is a randomly ethoxylated/propoxylated 1,4-butanediol vinyl ether having the empirical formula CH₂═CH—O(CH₂)₄O(C₃H₆O)₄(C₂H₄O)₁₀H; Emulsogen® R208 is a randomly ethoxylated/propoxylated 1,4-butanediol vinyl ether having the empirical formula CH₂═CH—O(CH₂)₄O(C₃H₆O)₄(C₂H₄O)₂₀H; Emulsogen® R307 is a randomly ethoxylated/propoxylated 1,4-butanediol vinyl ether having the empirical formula CH₂═CH—O(CH₂)₄O(C₃H₆O)₄(C₂H₄O)₃₀H; Emulsogen® RAL109 is a randomly ethoxylated/propoxylated allyl ether having the empirical formula CH₂═CHCH₂O(C₃H₆O)₄(C₂H₄O)₁₀H; Emulsogen® RAL208 is a randomly ethoxylated/propoxylated allyl ether having the empirical formula CH₂═CHCH₂O(C₃H₆O)₄(C₂H₄O)₂₀H; Emulsogen® RAL307 is a randomly ethoxylated/propoxylated allyl ether having the empirical formula CH₂═CHCH₂O(C₃H₆O)₄(C₂H₄O)₃₀H; and BX-AA-E5P5 is a randomly ethoxylated/propoxylated allyl ether having the empirical formula CH₂═CHCH₂O(C₃H₆O)₅(C₂H₄O)₅H.

Referring to the alkoxylated associative and the alkoxylated semi-hydrophobic monomers of the disclosed technology, the polyoxyalkylene mid-section portion contained in these monomers can be utilized to tailor the hydrophilicity and/or hydrophobicity of the polymers in which they are included. For example, mid-section portions rich in ethylene oxide moieties are more hydrophilic while mid-section portions rich in propylene oxide moieties are more hydrophobic. By adjusting the relative amounts of ethylene oxide to propylene oxide moieties present in these monomers the hydrophilic and hydrophobic properties of the polymers in which these monomers are included can be tailored as desired.

The amount of alkoxylated associative and/or semi-hydrophobic monomer utilized in the preparation of the polymers of the present disclosed technology can vary widely and depends, among other things, on the final rheological and aesthetic properties desired in the polymer. When utilized, the monomer reaction mixture contains one or more monomers selected from the alkoxylated associative and/or semi-hydrophobic monomers disclosed above in amounts ranging from about 0.5 to about 10 wt. % in one aspect, and from about 1, 2 or 3 to about 5 wt. % in a further aspect, based on the weight of the total monomers.

### Ionizable Monomer

In one aspect of the disclosed technology, the nonionic, amphiphilic polymer compositions of the disclosed technology can be polymerized from a monomer composition including 0 to 5 wt. % of an ionizable and/or ionized monomer, based on the weight of the total monomers, so long as the conditioning effect and the yield stress value of the surfactant compositions in which the polymers of the disclosed technology are included are not deleteriously affected.

In another aspect, the amphiphilic polymer compositions of the disclosed technology can be polymerized from a monomer composition comprising less than 3 wt. % in one aspect, less than 1 wt. % in a further aspect, less than 0.5 wt. % in a still further aspect, less than 0.1 wt. % in an additional aspect, and less than 0.05 wt. % in a further aspect, of an ionizable and/or an ionized moiety, based on the weight of the total monomers.

Ionizable monomers include monomers having a base neutralizable moiety and monomers having an acid neutralizable moiety. Base neutralizable monomers include olefinically unsaturated monocarboxylic and dicarboxylic acids and their salts containing 3 to 5 carbon atoms and anhydrides thereof. Examples include (meth)acrylic acid, itaconic acid, maleic acid, maleic anhydride, and combinations thereof. Other acidic monomers include styrenesulfonic acid, acrylamidomethylpropanesulfonic acid (AMPS® monomer), vinylsulfonic acid, vinylphosphonic acid, allylsulfonic acid, methallylsulfonic acid; and salts thereof.

Acid neutralizable monomers include olefinically unsaturated monomers which contain a basic nitrogen atom capable of forming a salt or a quaternized moiety upon the addition of an acid. For example, these monomers include vinylpyridine, vinylpiperidine, vinylimidazole, vinylmethylimidazole, dimethylaminomethyl (meth)acrylate, dimethylaminoethyl (meth)acrylate, diethylaminomethyl (meth)acrylate and methacrylate, dimethylaminoneopentyl (meth)acrylate, dimethylaminopropyl (meth)acrylate, and diethylaminoethyl (meth)acrylate.

### Crosslinking Monomer

In one aspect, the crosslinked, nonionic, amphiphilic polymers useful in the practice of the disclosed technology are polymerized from a monomer composition comprising a first monomer comprising at least one nonionic, hydrophilic unsaturated monomer, at least one nonionic, unsaturated hydrophobic monomer, and mixtures thereof, and a third monomer comprising at least one polyunsaturated crosslinking monomer. The crosslinking monomer(s) is utilized to polymerize covalent crosslinks into the polymer backbone. In one aspect, the crosslinking monomer is a polyunsaturated compound containing at least 2 unsaturated moieties. In another aspect, the crosslinking monomer contains at least 3 unsaturated moieties. Exemplary polyunsaturated compounds include di(meth)acrylate compounds such as ethylene glycol di(meth)acrylate, polyethylene glycol di(meth)acrylate, triethylene glycol di(meth)acrylate, 1,3-butylene glycol di(meth)acrylate, 1,6-butylene glycol di(meth)acrylate, 1,6-hexanediol di(meth)acrylate, neopentyl glycol di(meth)acrylate, 1,9-nonanediol di(meth)acrylate, 2,2′-bis(4-(acryloxy-propyloxyphenyl)propane, and 2,2′-bis(4-(acryloxydiethoxy-phenyl)propane; tri(meth)acrylate compounds such as, trimethylolpropane tri(meth)acrylate, trimethylolethane tri(meth)acrylate, and tetramethylolmethane tri(meth)acrylate; tetra(meth)acrylate compounds such as ditrimethylolpropane tetra(meth)acrylate, tetramethylolmethane tetra(meth)acrylate, and pentaerythritol tetra(meth)acrylate; hexa(meth)acrylate compounds such as dipentaerythritol hexa(meth)acrylate; allyl compounds such as allyl (meth)acrylate, diallylphthalate, diallyl itaconate, diallyl fumarate, and diallyl maleate; polyallyl ethers of sucrose having from 2 to 8 allyl groups per molecule, polyallyl ethers of pentaerythritol such as pentaerythritol diallyl ether, pentaerythritol triallyl ether, and pentaerythritol tetraallyl ether, and combinations thereof; polyallyl ethers of trimethylolpropane such as trimethylolpropane diallyl ether, trimethylolpropane triallyl ether, and combinations thereof. Other suitable polyunsaturated compounds include divinyl glycol, divinyl benzene, and methylenebisacrylamide.

In another aspect, suitable polyunsaturated monomers can be synthesized via an esterification reaction of a polyol made from ethylene oxide or propylene oxide or combinations thereof with unsaturated anhydride such as maleic anhydride, citraconic anhydride, itaconic anhydride, or an addition reaction with unsaturated isocyanate such as 3-isopropenyl-α-α-dimethylbenzene isocyanate.

Mixtures of two or more of the foregoing polyunsaturated compounds can also be utilized to crosslink the nonionic, amphiphilic polymers of the disclosed technology. In one aspect, the mixture of unsaturated crosslinking monomer contains an average of 2 unsaturated moieties. In another aspect, the mixture of crosslinking monomers contains an average of 2.5 unsaturated moieties. In still another aspect, the mixture of crosslinking monomers contains an average of about 3 unsaturated moieties. In a further aspect, the mixture of crosslinking monomers contains an average of about 3.5 unsaturated moieties. In one embodiment of the disclosed technology, the amount of the crosslinking monomer ranges from 0 to about 1 wt. % in one aspect, from about 0.01 to about 0.75 wt. % in another aspect, from about 0.1 to about 0.5 in still another aspect, and from about 0.15 to about 0.3 wt. % in a still further aspect, all weight percentages are based on the weight of the monomer composition.

In another embodiment of the disclosed technology, the crosslinking monomer component contains an average of about 3 unsaturated moieties and can be used in an amount ranging from about 0.01 to about 0.3 wt. % in one aspect, from about 0.02 to about 0.25 wt. % in another aspect, from about 0.05 to about 0.2 wt. % in a further aspect, and from about 0.075 to about 0.175 wt. % in a still further aspect, and from about 0.1 to about 0.15 wt. % in another aspect, based on the weight of the monomer composition.

In one aspect, the crosslinking monomer is selected from trimethylolpropane tri(meth)acrylate, trimethylolethane tri(meth)acrylate, tetramethylolmethane tri(meth)acrylate, pentaerythritol triallylether and polyallyl ethers of sucrose having 3 allyl groups per molecule.

In one aspect, the crosslinking monomer is selected from a reactive surfactant compound containing an average of at least two polymerizable unsaturated groups. In one aspect, the reactive surfactant conforms to the formula:

R₁O—(CH₂CHR₂O)ₓ—(CH₂CH₂O)_{y}—(CH₂CHR₃O)_{z}—R₄ X

wherein R₁ is selected from alkyl, aryl, alkylaryl, and aralkylaryl of 8-30 carbon atoms, R₂ is —CH₂OCH₂CH═CH₂; R₃ is selected from H, CH₃, and CH₂CH₃; R₄ is H or —SO₃⁻M⁻ or —PO₃⁻M⁻, where M is selected from H or K, Na, NH₄, and alkanolamine; and x=2 to 100; y=4 to 200 and z=0 or 1 to 50.

In one aspect, the reactive surfactant crosslinker conforms to the formula:

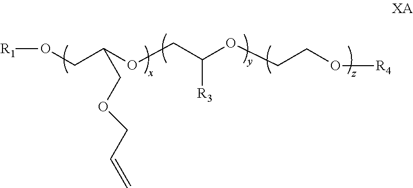

wherein R₁ is selected from a C₁₀₋₂₄ alkyl, alkaryl, alkenyl, or cycloalkyl, R₃ is selected from CH₃, CH₂CH₃, C₆H₅, and C₁₄H₂₉; x is 2-10; y is 0-200; and z is 4-200 in one aspect, 5 to 60 in another aspect, and 5 to 40 in a further aspect; and R₄ is selected from hydrogen, SO₃⁻M⁺ or PO₃⁻M⁺, and M is Na, K, NH₄, and an alkanolamine, such as monoethanolamine, diethanolamine, and triethanolamine.

In one aspect, the reactive surfactant crosslinker conforms to the formula:

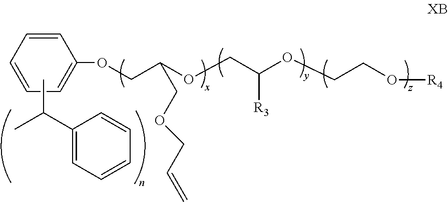

wherein R₃ is selected from CH₃, CH₂CH₃, C₆H₅, and C₁₄H₂₉; n is 1, 2, or 3; x is 2 to 10; y is 0 or 1 to 200; and z is 4 to 200 in one aspect, 5 to 60 in another aspect, and 5 to 40 in a further aspect; and R₄ is selected from hydrogen, SO₃⁻M⁺ or PO₃⁻M⁺, and M is Na, K, NH₄, and an alkanolamine, such as monoethanolamine, diethanolamine, and triethanolamine.

In one aspect, the reactive surfactant crosslinker conforms to the formula:

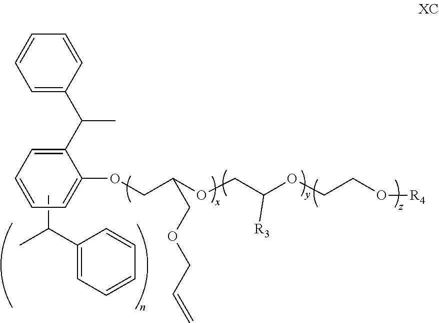

wherein R₃ is selected from CH₃, CH₂CH₃, C₆H₅, and C₁₄H₂₉; n is 1, or 2; x is 2 to 10; y is 0 or 1 to 200; and z is 4 to 200 in one aspect, 5 to 60 in another aspect, and 5 to 40 in a further aspect; and R⁴ is selected from hydrogen, SO₃⁻M⁺ or PO₃⁻M⁺, and M is Na, K, NH₄, and an alkanolamine, such as monoethanolamine, diethanolamine, and triethanolamine.

In one aspect, the reactive surfactant crosslinker conforms to the formula:

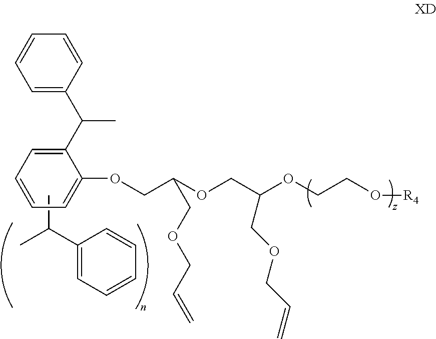

where n is 1 or 2; z is 4 to 40 in one aspect, 5 to 38 in another aspect, and 10 to 20 in a further aspect; and R₄ is SO₃⁻M⁺ or PO₃⁻M⁺, and M is selected from Na, K, and NH₄.

The reactive surfactant crosslinkers conforming to Formulas X, XA, XB, XC, and XD are disclosed in U.S. Patent Application Publication No. US 2014/0114006, the disclosure of which is herein incorporated by reference. The reactive surfactant crosslinkers disclosed under the foregoing formulas (Formulas X through XD) are commercially available under the E-Sperse™ RS Series trade name (e.g., product designations RS-1617, RS-1618, RS-1684) of reactive surfactants from Ethox Chemicals, LLC.

The amount of reactive surfactant that is utilized to crosslink the nonionic, amphiphilic polymers useful in the practice of the disclosed technology ranges from about 0.1 to about 5 wt. % in one aspect, from about 0.5 to about 3 wt. % in another aspect, and from about 0.75 to about 1.5 wt. % in a further aspect (based on the total weight of monomers).

Combinations of the crosslinking monomers and crosslinking reactive surfactant monomers can be utilized in the practice of the disclosed technology.

### Amphiphilic Polymer Synthesis

The linear and crosslinked, nonionic, amphiphilic, irritation mitigant polymers of the disclosed technology can be made using conventional free-radical emulsion polymerization techniques. The polymerization processes are carried out in the absence of oxygen under an inert atmosphere such as nitrogen. The polymerization can be carried out in a suitable solvent system such as water. Minor amounts of a hydrocarbon solvent, organic solvent, as well as mixtures thereof can be employed. The polymerization reactions are initiated by any means which results in the generation of a suitable free-radical. Thermally derived radicals, in which the radical species is generated from thermal, homolytic dissociation of peroxides, hydroperoxides, persulfates, percarbonates, peroxyesters, hydrogen peroxide and azo compounds can be utilized. The initiators can be water soluble or water insoluble depending on the solvent system employed for the polymerization reaction.

The initiator compounds can be utilized in an amount of up to 30 wt. % in one aspect, 0.01 to 10 wt. % in another aspect, and 0.2 to 3 wt. % in a further aspect, based on the total weight of the dry polymer.

Exemplary free radical water soluble initiators include, but are not limited to, inorganic persulfate compounds, such as ammonium persulfate, potassium persulfate, and sodium persulfate; peroxides such as hydrogen peroxide, benzoyl peroxide, acetyl peroxide, and lauryl peroxide; organic hydroperoxides, such as cumene hydroperoxide and t-butyl hydroperoxide; organic peracids, such as peracetic acid, and water soluble azo compounds, such as 2,2′-azobis(tert-alkyl) compounds having a water solubilizing substituent on the alkyl group. Exemplary free radical oil soluble compounds include, but are not limited to 2,2′-azobisisobutyronitrile, and the like. The peroxides and peracids can optionally be activated with reducing agents, such as sodium bisulfite, sodium formaldehyde, or ascorbic acid, transition metals, hydrazine, and the like.

In one aspect, azo polymerization catalysts include the Vazo® free-radical polymerization initiators, available from DuPont, such as Vazo® 44 (2,2′-azobis(2-(4,5-dihydroimidazolyl)propane), Vazo® 56 (2,2′-azobis(2-methylpropionamidine) dihydrochloride), Vazo® 67 (2,2′-azobis(2-methylbutyronitrile)), and Vazo® 68 (4,4′-azobis(4-cyanovaleric acid)).

In emulsion polymerization processes, it can be advantageous to stabilize the monomer/polymer droplets or particles by means of surface active auxiliaries. Typically, these are emulsifiers or protective colloids. Emulsifiers used can be anionic, nonionic, cationic or amphoteric. Examples of anionic emulsifiers are alkylbenzenesulfonic acids, sulfonated fatty acids, sulfosuccinates, fatty alcohol sulfates, alkylphenol sulfates and fatty alcohol ether sulfates. Examples of usable nonionic emulsifiers are alkylphenol ethoxylates, primary alcohol ethoxylates, fatty acid ethoxylates, alkanolamide ethoxylates, fatty amine ethoxylates, EO/PO block copolymers and alkylpolyglucosides. Examples of cationic and amphoteric emulsifiers used are quaternized amine alkoxylates, alkylbetaines, alkylamidobetaines and sulfobetaines.

Optionally, the use of known redox initiator systems as polymerization initiators can be employed. Such redox initiator systems include an oxidant (intiator) and a reductant. Suitable oxidants include, for example, hydrogen peroxide, sodium peroxide, potassium peroxide, t-butyl hydroperoxide, t-amyl hydroperoxide, cumene hydroperoxide, sodium perborate, perphosphoric acid and salts thereof, potassium permanganate, and ammonium or alkali metal salts of peroxydisulfuric acid, typically at a level of 0.01% to 3.0% by weight, based on dry polymer weight, are used. Suitable reductants include, for example, alkali metal and ammonium salts of sulfur-containing acids, such as sodium sulfite, bisulfite, thiosulfate, hydrosulfite, sulfide, hydrosulfide or dithionite, formadinesulfinic acid, hydroxymethanesulfonic acid, acetone bisulfite, amines such as ethanolamine, glycolic acid, glyoxylic acid hydrate, ascorbic acid, isoascorbic acid, lactic acid, glyceric acid, malic acid, 2-hydroxy-2-sulfinatoacetic acid, tartaric acid and salts of the preceding acids typically at a level of 0.01% to 3.0% by weight, based on dry polymer weight, is used. In one aspect, combinations of peroxodisulfates with alkali metal or ammonium bisulfites can be used, for example, ammonium peroxodisulfate and ammonium bisulfite. In another aspect, combinations of hydrogen peroxide containing compounds (t-butyl hydroperoxide) as the oxidant with ascorbic or erythorbic acid as the reductant can be utilized. The ratio of peroxide-containing compound to reductant is within the range from 30:1 to 0.05:1.

Examples of typical protective colloids are cellulose derivatives, polyethylene glycol, polypropylene glycol, copolymers of ethylene glycol and propylene glycol, polyvinyl acetate, poly(vinyl alcohol), partially hydrolyzed poly(vinyl alcohol), polyvinyl ether, starch and starch derivatives, dextran, polyvinylpyrrolidone, polyvinylpyridine, polyethyleneimine, polyvinylimidazole, polyvinylsuccinimide, polyvinyl-2-methylsuccinimide, polyvinyl-1,3-oxazolid-2-one, polyvinyl-2-methylimidazoline and maleic acid or anhydride copolymers. The emulsifiers or protective colloids are customarily used in concentrations from 0.05 to 20 wt. %, based on the weight of the total monomers.

The polymerization reaction can be carried out at temperatures ranging from 20 to 200° C. in one aspect, from 50 to 150° C. in another aspect, and from 60 to 100° C. in a further aspect.

The polymerization can be carried out the presence of chain transfer agents. Suitable chain transfer agents include, but are not limited to, thio- and disulfide containing compounds, such as C₁-C₁₈ alkyl mercaptans, such as tert-butyl mercaptan, n-octyl mercaptan, n-dodecyl mercaptan, tert-dodecyl mercaptan hexadecyl mercaptan, octadecyl mercaptan; mercaptoalcohols, such as 2-mercaptoethanol, 2-mercaptopropanol; mercaptocarboxylic acids, such as mercaptoacetic acid and 3-mercaptopropionic acid; mercaptocarboxylic acid esters, such as butyl thioglycolate, isooctyl thioglycolate, dodecyl thioglycolate, isooctyl 3-mercaptopropionate, and butyl 3-mercaptopropionate; thioesters; C₁-C₁₈ alkyl disulfides; aryldisulfides; polyfunctional thiols such as trimethylolpropane-tris-(3-mercaptopropionate), pentaerythritol-tetra-(3-mercaptopropionate), pentaerythritol-tetra-(thioglycolate), pentaerythritol-tetra-(thiolactate), dipentaerythritol-hexa-(thioglycolate), and the like; phosphites and hypophosphites; C₁-C₄ aldehydes, such as formaldehyde, acetaldehyde, propionaldehyde; haloalkyl compounds, such as carbon tetrachloride, bromotrichloromethane, and the like; hydroxylammonium salts such as hydroxylammonium sulfate; formic acid; sodium bisulfite; isopropanol; and catalytic chain transfer agents such as, for example, cobalt complexes (e.g., cobalt (II) chelates).

The chain transfer agents are generally used in amounts ranging from 0.1 to 10 wt. %, based on the total weight of the monomers present in the polymerization medium.

### Emulsion Process

In one exemplary aspect of the disclosed technology, the crosslinked, nonionic, amphiphilic polymer is polymerized via an emulsion process. The emulsion process can be conducted in in a single reactor or in multiple reactors as is well-known in the art. The monomers can be added as a batch mixture or each monomer can be metered into the reactor in a staged process. A typical mixture in emulsion polymerization comprises water, monomer(s), an initiator (usually water-soluble) and an emulsifier. The monomers may be emulsion polymerized in a single-stage, two-stage or multi-stage polymerization process according to well-known methods in the emulsion polymerization art. In a two-stage polymerization process, the first stage monomers are added and polymerized first in the aqueous medium, followed by addition and polymerization of the second stage monomers. The aqueous medium optionally can contain an organic solvent. If utilized the organic solvent is less than about 5 wt. % of the aqueous medium. Suitable examples of water-miscible organic solvents include, without limitation, esters, alkylene glycol ethers, alkylene glycol ether esters, lower molecular weight aliphatic alcohols, and the like.

To facilitate emulsification of the monomer mixture, the emulsion polymerization is carried out in the presence of at least one surfactant. In one embodiment, the emulsion polymerization is carried out in the presence of surfactant (active weight basis) ranging in the amount of about 0.2% to about 5% by weight in one aspect, from about 0.5% to about 3% in another aspect, and from about 1% to about 2% by weight in a further aspect, based on a total monomer weight basis. The emulsion polymerization reaction mixture also includes one or more free radical initiators which are present in an amount ranging from about 0.01% to about 3% by weight based on total monomer weight. The polymerization can be performed in an aqueous or aqueous alcohol medium. Surfactants for facilitating the emulsion polymerization include anionic, nonionic, amphoteric, and cationic surfactants, as well as mixtures thereof. Most commonly, anionic and nonionic surfactants can be utilized as well as mixtures thereof.

Suitable anionic surfactants for facilitating emulsion polymerizations are well known in the art and include, but are not limited to (C₆-C₁₈) alkyl sulfates, (C₆-C₁₈) alkyl ether sulfates (e.g., sodium lauryl sulfate and sodium laureth sulfate), amino and alkali metal salts of dodecylbenzenesulfonic acid, such as sodium dodecyl benzene sulfonate and dimethylethanolamine dodecylbenzenesulfonate, sodium (C₆-C₁₆) alkyl phenoxy benzene sulfonate, disodium (C₆-C₁₆) alkyl phenoxy benzene sulfonate, disodium (C₆-C₁₆) di-alkyl phenoxy benzene sulfonate, disodium laureth-3 sulfosuccinate, sodium dioctyl sulfosuccinate, sodium di-sec-butyl naphthalene sulfonate, disodium dodecyl diphenyl ether sulfonate, disodium n-octadecyl sulfosuccinate, phosphate esters of branched alcohol ethoxylates, and the like.

Nonionic surfactants suitable for facilitating emulsion polymerizations are well known in the polymer art, and include, without limitation, linear or branched C₈-C₃₀ fatty alcohol ethoxylates, such as capryl alcohol ethoxylate, lauryl alcohol ethoxylate, myristyl alcohol ethoxylate, cetyl alcohol ethoxylate, stearyl alcohol ethoxylate, cetearyl alcohol ethoxylate, sterol ethoxylate, oleyl alcohol ethoxylate, and, behenyl alcohol ethoxylate; alkylphenol alkoxylates, such as octylphenol ethoxylates; and polyoxyethylene polyoxypropylene block copolymers, and the like. Additional fatty alcohol ethoxylates suitable as non-ionic surfactants are described below. Other useful nonionic surfactants include C₈-C₂₂ fatty acid esters of polyoxyethylene glycol, ethoxylated mono- and diglycerides, sorbitan esters and ethoxylated sorbitan esters, C₈-C₂₂ fatty acid glycol esters, block copolymers of ethylene oxide and propylene oxide, and combinations thereof. The number of ethylene oxide units in each of the foregoing ethoxylates can range from 2 and above in one aspect, and from 2 to about 150 in another aspect.

Optionally, other emulsion polymerization additives and processing aids which are well known in the emulsion polymerization art, such as auxiliary emulsifiers, protective colloids, solvents, buffering agents, chelating agents, inorganic electrolytes, polymeric stabilizers, biocides, and pH adjusting agents can be included in the polymerization system.

In one aspect of the disclosed technology, the protective colloid or auxiliary emulsifier is selected from poly(vinyl alcohol) that has a degree of hydrolysis ranging from about 80 to 95% in one aspect, and from about 85 to 90% in another aspect.

In a typical two stage emulsion polymerization, a mixture of the monomers is added to a first reactor under inert atmosphere to a solution of emulsifying surfactant (e.g., anionic surfactant) in water. Optional processing aids can be added as desired (e.g., protective colloids, auxiliary emulsifier(s)). The contents of the reactor are agitated to prepare a monomer emulsion. To a second reactor equipped with an agitator, an inert gas inlet, and feed pumps are added under inert atmosphere a desired amount of water and additional anionic surfactant and optional processing aids. The contents of the second reactor are heated with mixing agitation. After the contents of the second reactor reaches a temperature in the range of about 55 to 98° C., a free radical initiator is injected into the so formed aqueous surfactant solution in the second reactor, and the monomer emulsion from the first reactor is gradually metered into the second reactor over a period typically ranging from about one half to about four hours. The reaction temperature is controlled in the range of about 45 to about 95° C. After completion of the monomer addition, an additional quantity of free radical initiator can optionally be added to the second reactor, and the resulting reaction mixture is typically held at a temperature of about 45 to 95° C. for a time period sufficient to complete the polymerization reaction to obtain the polymer emulsion.

In one embodiment, the crosslinked, nonionic, amphiphilic polymers of the disclosed technology are selected from an emulsion polymer polymerized from a monomer mixture comprising at least 30 wt. % of at least one C₁-C₄ hydroxyalkyl (meth)acrylate (e.g., hydroxyethyl methacrylate), 15 to 70 wt. % of at least one C₁-C₁₂ alkyl acrylate, 0 to 40 wt. % or 5 to 40 wt. % of at least one vinyl ester of a C₁-C₁₀ carboxylic acid (based on the weight of the total monomers), and 0.01 to 1 wt. % at least one crosslinker (based on the weight of the monomer composition.

In another aspect, the crosslinked, nonionic, amphiphilic polymers of the disclosed technology are selected from an emulsion polymer polymerized from a monomer mixture comprising at least 30 wt. % hydroxyethyl methacrylate, 15 to 35 wt. % ethyl acrylate, 5 to 25 wt. % butyl acrylate, 0 to 25 wt. % or 10 to 25 wt. % of a vinyl ester of a C₁-C₅ carboxylic acid selected from vinyl, acetate, vinyl propionate, vinyl butyrate, vinyl isobutyrate, and vinyl valerate (said weight percent is based on the weight of the total monomers), and from about 0.01 to about 0.3 wt. % of a crosslinking monomer having an average of at least 3 crosslinkable unsaturated groups (based on the weight of the monomer composition).

In another embodiment, the crosslinked, nonionic, amphiphilic polymers of the disclosed technology are selected from an emulsion polymer polymerized from a monomer mixture comprising from about 30 to 60 wt. % of at least one C₁-C₄ hydroxyalkyl (meth)acrylate (e.g., hydroxyethyl methacrylate), 15 to 70 wt. % of at least one C₁-C₁₂ alkyl acrylate (at least one C₁-C₅ alkyl acrylate in another aspect), from about 0.1 to about 10 wt. of at least one associative and/or semi-hydrophobic monomer (based on the weight of the total monomers), and from 0.01 to about 1 wt. % at least one crosslinker (based on the weight of the monomer composition).

In another embodiment, the crosslinked, nonionic, amphiphilic polymers of the disclosed technology are selected from an emulsion polymer polymerized from a monomer mixture comprising from about 35 to 50 wt. % of at least one C₁-C₄ hydroxyalkyl (meth)acrylate (e.g., hydroxyethyl methacrylate), 15 to 60 wt. % of at least one C₁-C₁₂ alkyl acrylate (at least one C₁-C₅ alkyl acrylate in another aspect), from about 0.1 to about 10 wt. % of at least one associative and/or semi-hydrophobic monomer (based on the weight of the total monomers), and from 0.01 to about 1 wt. % at least one crosslinker (based on the weight of the monomer composition).

In one aspect, the crosslinked, nonionic, amphiphilic polymers of the disclosed technology are selected from an emulsion polymer polymerized from a monomer mixture comprising from about 40 to 45 wt. % of at least one C₁-C₄ hydroxyalkyl (meth)acrylate (e.g., hydroxyethyl methacrylate), 15 to 60 wt. % of at least two different C₁-C₅ alkyl acrylate monomers, from about 1 to about 5 wt. % of at least one associative and/or semi-hydrophobic monomer (based on the weight of the total monomers), and from 0.01 to about 1 wt. % at least one crosslinker (based on the weight of the monomer composition).

In one aspect, the crosslinked, nonionic, amphiphilic polymers of the disclosed technology are selected from an emulsion polymer polymerized from a monomer mixture comprising from about 40 to 45 wt. % of hydroxyethyl acrylate, 30 to 50 wt. % of ethyl acrylate, 10 to 20 wt. % of butyl acrylate and from about 1 to about 5 wt. % of at least one associative and/or semi-hydrophobic monomer (based on the weight of the total monomers), and from 0.01 to about 1 wt. % at least one crosslinker (based on the weight of the dry polymer).

In one aspect, the at least one nonionic, amphiphilic polymer utilized in formulating the hair conditioning compositions of the disclosed technology is a linear polymer. In one aspect, the number average molecular weight (Mn) of the linear copolymer of the disclosed technology as measured by gel permeation chromatography (GPC) calibrated with a poly(methyl methacrylate) (PMMA) standard is 500,000 daltons or less. In another aspect the molecular weight is 100,000 daltons or less. In still another aspect, the molecular weight ranges between about 5,000 and about 80,000 daltons, in a further aspect between about 10,000 and 50,000 daltons, and in a still further aspect between about 15,000 and 40,000 daltons.

The crosslinked nonionic, amphiphilic polymers of the technology are random copolymers and have weight average molecular weights ranging from above about 500,000 to at least about a billion Daltons or more in one aspect, and from about 600,000 to about 4.5 billion Daltons in another aspect, and from about 1,000,000 to about 3,000,000 Daltons in a further aspect, and from about 1,500,000 to about 2,000,000 Daltons in a still further aspect (see TDS-222, Oct. 15, 2007, Lubrizol Advanced Materials, Inc., which is herein incorporated by reference).

The amount of the linear or crosslinked nonionic, amphiphilic polymer (active material basis) utilized in the compositions of the present technology ranges from about 0.5 to about 6.5 wt. % in one aspect, from about 1 to about 5 wt. % in another aspect, and from about 2 to about 4 wt. % in still another aspect, based on the weight of the total composition.

### Aqueous Carrier

The hair conditioning composition of the present technology comprises an aqueous carrier component (d) which is substantially water. In one aspect, the aqueous carrier can include other solvents that are miscible with water. In one aspect, the water miscible solvents include lower alkyl alcohols and polyhydric alcohols. The lower alkyl alcohols useful herein are monohydric alcohols having 1 to 6 carbon atoms including ethanol and isopropanol. The polyhydric alcohols include, for example, alkylene glycols having about 2 to about 5 carbon atoms per molecule (e.g., propylene glycol, ethylene glycol, butylene glycol), propanediol (e.g., 1,2-propanediol, 1,3-propanediol) and butanediol, as well as dialkylene glycols (e.g., diethylene and dipropylene glycols).

In one aspect, the substantially aqueous carrier component comprises deionized water, although water from natural, municipal or commercial sources can be utilized as long as any mineral cations that may be present in such water do not deleteriously affect the intended function of any of the components contained in the present hair conditioning composition. By substantially aqueous is meant that the aqueous carrier component contains from about 100 to about 80 wt. % in one aspect, from about 85 to about 99 wt. % in another aspect, and from about 90 to about 95 wt. % water in still another aspect, with the remaining wt. % selected from at least one water miscible solvent mentioned above.

Generally, the aqueous carrier component is present in the hair conditioning composition an amount ranging from about 20 to about 95 wt. % in one aspect, from about 30 to 90 wt. % in another aspect, and from about 50 to 85 wt. % in still another aspect, based on the weight of the total composition.

### Auxiliary Conditioning Agent

The compositions of the present technology can contain auxiliary conditioning agents in addition to the cationic surfactant component (a). These optional auxiliary conditioning agents include any material which is used to give a particular conditioning benefit to hair, scalp and/or skin. Suitable conditioning agents are those which deliver one or more benefits relating to shine, softness, combability (wet and/or dry), antistatic properties, wet-handling, damage, manageability, body, and volume. Exemplary classes of auxiliary conditioning agents for use in the composition are characterized generally as silicones (e.g., silicone oils, cationic silicones, silicone gums, high refractive silicones, silicone resins and dimethicone copolyols), organic conditioning oils (e.g., hydrocarbon oils, polyolefin oils, fluorinated oils, natural oils, and fatty ester oils), cationic compounds (different than (a)), cationic polymers, and combinations thereof. Such conditioning agents should be physically and chemically compatible with the components of the composition, and should not otherwise unduly impair product stability, aesthetics or performance.

### A. Silicones

In one aspect of the present technology, the hair conditioning composition optionally contains a silicone conditioning agent. The silicone conditioning agent may comprise volatile silicones, non-volatile silicones, water soluble silicones (e.g., dimethicone copolyols), and mixtures thereof. If volatile silicones are present, they are typically employed as a solvent or carrier for commercially available forms of non-volatile silicone fluid conditioning agents such as oils and gums and resins. Volatile silicone fluids are often included in the conditioning package to improve silicone fluid deposition efficacy or to enhance the shine, sheen or glossiness of the hair. Volatile silicone materials are frequently included in formulations to enhance sensory attributes (e.g., feel) on the hair, scalp and skin.

In one aspect, the silicone conditioning agent is non-volatile and includes silicone oils, gums, resins and mixtures thereof. By non-volatile is meant that the silicone has a very low vapor pressure at ambient temperature conditions (e.g., less than 2 mm Hg at 20° C.). The non-volatile silicone conditioning agent has a boiling point above about 250° C. in one aspect, above about 260° C. in another aspect, and above about 275° C. in a further aspect. Background information on silicones including sections discussing silicone oils, gums, and resins, as well as their manufacture, are found in Encyclopedia of Polymer Science and Engineering, vol. 15, 2d ed., pp 204-308, John Wiley & Sons, Inc. (1989).

In one aspect, the silicone oil conditioning agent includes amino functional and quaternized polyorganosiloxanes represented by the formula:

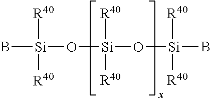

wherein B independently represents hydroxy, methyl, methoxy, ethoxy, propoxy, and phenoxy; R⁴⁰ independently represents methyl, ethyl, propyl, phenyl, methylphenyl, phenylmethyl, a primary, secondary or tertiary amine, a quaternary group selected from a group selected from:

—R⁴¹—N(R⁴²)CH₂CH₂N(R⁴²)₂;

—R⁴¹—N(R⁴²)₂;

—R⁴¹—N⁺(R⁴²)₃CA⁻; and

—R⁴¹—N(R⁴²)CH₂CH₂N⁺(R⁴²)H₂CA⁻

wherein R⁴¹ is a linear or branched, hydroxyl substituted or unsubstituted alkylene or alkylene ether moiety containing 2 to 10 carbon atoms; R⁴² independently is hydrogen, C₁-C₂₀ alkyl (e.g, methyl), phenyl or benzyl; q is an integer ranging from about 2 to about 8; CA⁻ is a halide ion selected from chlorine, bromine, iodine and fluorine; and x is an integer ranging from about 7 to about 8000 in one aspect, from about 50 to about 5000 in another aspect, form about 100 to about 3000 in still another aspect, and from about 200 to about 1000 in a further aspect.

In one aspect, the amino functional and quaternized polyalkylsiloxane can be represented by the formula:

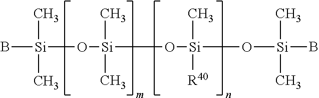

wherein B independently represents hydroxy, methyl, methoxy, ethoxy, propoxy, and phenoxy; and R⁴⁰ is selected from:

—R⁴¹—N(R⁴²)CH₂CH₂N(R⁴²)₂;

—R⁴¹—N(R⁴²)₂;

—R⁴¹—N⁺(R⁴²)₃CA⁻; and

—R⁴¹—N(R⁴²)CH₂CH₂N⁺(R⁴²)H₂CA⁻

wherein R⁴¹ is a linear or branched, hydroxyl substituted or unsubstituted alkylene or alkylene ether moiety containing 2 to 10 carbon atoms; R⁴² independently is hydrogen, C₁-C₂₀ alkyl, phenyl or benzyl; CA⁻ is a halide ion selected from chlorine, bromine, iodine and fluorine; and the sum of m+n ranges from about 7 to about 1000 in one aspect, from about 50 to about 250 in another aspect, and from about 100 to about 200 in another aspect, subject to the proviso that m or n is not 0. In one aspect, B is hydroxy and R⁴⁰ is —(CH₂)₃NH(CH₂)₃NH₂. In another aspect, B is methyl and R⁴⁰ is —(CH₂)₃NH(CH₂)₃NH₂. In still another aspect, B is methyl and R⁴⁰ is a quaternary ammonium moiety represented by —(CH₂)₃OCH₂CH(OH)CH₂N⁺(R⁴²)₃ CA⁻; wherein R⁴² and CA⁻ are as previously defined.

The silicone oil conditioning agents can have a viscosity ranging from about above about 25 to about 1,000,000 mPa·s at 25° C. in one aspect, from about 100 to about 600,000 mPa·s in another aspect, and from about 1000 to about 100,000 mPa·s still another aspect, from about 2,000 to about 50,000 mPa·s in yet another aspect, and from about 4,000 to about 40,000 mPa·s in a further aspect. The viscosity is measured by means of a glass capillary viscometer as described by Dow Corning Corporate Test Method CTM004, dated Jul. 20, 1970. In one aspect the silicone oils have an average molecular weight below about 200,000 daltons. The average molecular weight can typically range from about 400 to about 199,000 daltons in one aspect, from about 500 to about 150,000 daltons in another aspect, from about 1,000 to about 100,000 daltons in still another aspect, from about 5,000 to about 65,000 daltons in a further aspect.

Exemplary silicone oil conditioning agents include, but are not limited to, polydimethylsiloxanes (dimethicones), polydiethylsiloxanes, polydimethyl siloxanes having terminal hydroxyl groups (dimethiconols), polymethylphenylsiloxanes, phenylmethylsiloxanes, amino functional polydimethylsiloxanes (amodimethicones), and mixtures thereof.

In addition to the quaternized silicone conditioning agents set forth under the formulas above, suitable non-limiting examples (which may or may not fall under the above formulas are Silicone Quaternium-1, Silicone Quaternium-2, Silicone Quaternium-2 Panthenol Succinate, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-16/Glycidoxy Dimethicone Crosspolymer, Silicone Quaternium-17, Silicone Quaternium-18, Silicone Quaternium-20, Silicone Quaternium-21 and Quaternium-80.

Another silicone conditioning agent useful in the disclosed technology is a silicone gum. A silicone gum is a polyorganosiloxane material of the same general structure of the silicone oils set forth above wherein B independently represents hydroxy, methyl, methoxy, ethoxy, propoxy, and phenoxy; R⁴⁰ independently represents methyl, ethyl, propyl, phenyl, methylphenyl, phenylmethyl, and vinyl. Silicone gums have a viscosity measured at 25° C. of greater than 1,000,000 mPa·s. The viscosity can be measured by means of a glass capillary viscometer as described above for the silicone oils. In one aspect the silicone gums have an average molecular weight about 200,000 daltons and above. The molecular weight can typically range from about 200,000 to about 1,000,000 daltons. It is recognized that the silicone gums described herein can also have some overlap with the silicone oils described previously. This overlap is not intended as a limitation on any of these materials.

Suitable silicone gums for use in the silicone component of compositions of the disclosed technology are polydimethylsiloxanes (dimethicones), optionally having terminal end groups such as hydroxyl (dimethiconols), polymethylvinylsiloxane, polydiphenylsiloxane, and mixtures thereof.

Silicone resins can be included as a silicone conditioning agent suitable for use in the compositions of the disclosed technology. These resins are crosslinked polysiloxanes. The crosslinking is introduced through the incorporation of trifunctional and tetrafunctional silanes with monofunctional and/or difunctional silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetra-functional siloxane monomer units (and hence, a sufficient level of crosslinking) such that they form a rigid or hard film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. In one aspect, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinyl-chlorosilanes, and terachlorosilane, with the methyl substituted silanes being most commonly utilized.

Silicone materials and silicone resins can be identified according to a shorthand nomenclature system known to those of ordinary skill in the art as “MDTQ” nomenclature. Under this naming system, the silicone is described according to the presence of various siloxane monomer units which make up the silicone. The “MDTQ” nomenclature system is described in the publication entitled “Silicones: Preparation, Properties and Performance”; Dow Corning Corporation, 2005, and in U.S. Pat. No. 6,200,554.

Exemplary silicone resins for use in the compositions of the disclosed technology include, but are not limited to MQ, MT, MTQ, MDT and MDTQ resins. In one aspect, methyl is the silicone resin substituent. In another aspect, the silicone resin is selected from a MQ resins, wherein the M:Q ratio is from about 0.5:1.0 to about 1.5:1.0 and the average molecular weight of the silicone resin is from about 1000 to about 10,000 daltons.

The optional volatile silicones referred to above include linear polydimethylsiloxanes and cyclic polydimethylsiloxanes (cyclomethicones), and mixtures thereof. Volatile linear polydimethylsiloxanes (dimethicones) typically contain about 2 to about 9 silicon atoms, alternating with oxygen atoms in a linear arrangement. Each silicon atom is also substituted with two alkyl groups (the terminal silicon atoms are substituted with three alkyl groups), such as, for example, methyl groups. The cyclomethicones typically contain about 3 to about 7 dimethyl substituted silicon atoms in one aspect and from about 3 to about 5 in another aspect, alternating with oxygen atoms, in a cyclic ring structure. The term “volatile” means that the silicone has a measurable vapor pressure, or a vapor pressure of at least 2 mm of Hg at 20° C. The volatile silicones have a viscosity of 25 mPa·s or less at 25° C. in one aspect, from about 0.65 about to about 10 mPa·s in another aspect, from about 1 to about 5 mPa·s in still another aspect, and from about 1.5 to about 3.5 mPa·s in a further aspect. A description of linear and cyclic volatile silicones is found in Todd and Byers, “Volatile Silicone Fluids for Cosmetics”, Cosmetics and Toiletries, Vol. 91(1), pp. 27-32 (1976), and in Kasprzak, “Volatile Silicones”, Soap/Cosmetics/Chemical Specialties, pp. 40-43 (December 1986).

Exemplary volatile linear dimethicones include, but are not limited to, hexamethyldisiloxane, octamethyltrisiloxane, decamethyltetrasiloxane, dodecamethylpentasiloxane and blends thereof. Volatile linear dimethicones and dimethicone blends are commercially available from Dow Corning Corporation as Dow Corning 200® Fluid (e.g., product designations 0.65 CST, 1 CST, 1.5 CST, and 2 CST) and Dow Corning® 2-1184 Fluid.

Exemplary volatile cyclomethicones are D4 cyclomethicone (octamethylcyclotetrasiloxane), D5 cyclomethicone (decamethylcyclopentasiloxane), D6 cyclomethicone, and blends thereof (e.g., D4/D5 and D5/D6). Volatile cyclomethicones and cyclomethicone blends are commercially available from Momentive Performance Materials Inc. as SF1173, SF1202, SF1256, and SF1258 silicone fluids, and Dow Corning Corporation as Dow Corning® 244, 245, 246, 345, and 1401 silicone fluids. Blends of volatile cyclomethicones and volatile linear dimethicones also can be employed.

The amount of silicone conditioner(s) in the compositions of the present technology should be sufficient to provide the desired conditioning performance to the hair, and generally ranges from about 0.01 to about 20 wt. % in one aspect, from about 0.05 to about 15 wt. % in another aspect, from about 0.1% to about 10 wt. % in still another aspect, and from about 1 to about 5 wt. % in a further aspect, based on the total weight of the composition.

Another class of silicone conditioning agent useful in the disclosed technology is a dimethicone copolyol. The dimethicone copolyols are linear or branched copolymers of dimethylsiloxane (dimethicone) modified with terminal and/or pendant alkylene oxide units. Suitable alkylene oxide units are selected from ethylene oxide, propylene oxide, butylene oxide, and combinations thereof. When mixtures of alkylene oxides are present, the alkylene oxide units can be arranged as random or block segments. Dimethicone copolyols can be water soluble or oil soluble depending on the amount and type of polyalkylene oxide present in the dimethicone polymer. The dimethicone copolyols can be derivatized to be anionic, cationic, amphoteric or nonionic in character.

In one aspect, the nonionic dimethicone copolyol contains pendant polyoxyalkylene moieties and can be represented by the formula:

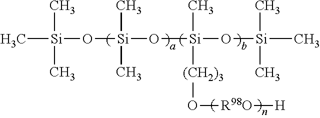

wherein a represents an integer ranging from about 0 or 1 to about 500; b is an integer ranging from about 1 to about 100; (R⁹⁸O)ₙ is a polyoxyalkylene moiety which can be arranged as a homopolymer, a random copolymer, or a block copolymer of oxyalkylene units; R⁹⁸ is a divalent alkylene moiety selected from C₂H₄, C₃H₆, or C₄H₈, and combinations thereof; and n is an integer ranging from about 1 to about 50 in one aspect, from about 3 to about 35 in another aspect, from about 5 to about 25 in a still another aspect, and from about 8 to about 20 in a further aspect.

Exemplary nonionic dimethicone copolyols containing pendant polyoxyalkylene moieties are commercially available under the Silsoft® trade name from Momentive Performance Materials. Specific product designations include, but are not limited to, Silsoft product designations 430 and 440 (PEG/PPG 20/23 Dimethicone), 475 (PEG/PPG 20/6 Dimethicone), 805 (PEG-8 Dimethicone), 875 and, 880 (PEG-12 Dimethicone), 895 (PEG-17 Dimethicone), and 910 (PPG-12 Dimethicone). Other commercially available dimethicone copolyols include Silsense™ Copolyol-1 a dimethicone copolyol blend (PEG-33 Dimethicone and PEG-8 Dimethicone and PEG-14 Dimethicone) from Lubrizol Advanced Materials, Inc.

In another aspect, the nonionic dimethicone copolyol contains terminal polyoxyalkylene moieties and can be represented by the formula:

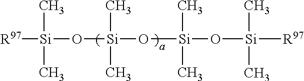

wherein R⁹⁷ independently is selected from methyl and the radical —(CH₂)ₘ—O—(R⁹⁸O)ₙ—H; a is an integer ranging from about 1 to about 500; (R⁹⁸O)ₙ is a polyoxyalkylene moiety which can be arranged as a homopolymer, a random copolymer, or a block copolymer of oxyalkylene units; R⁹⁸ is a divalent alkylene moiety selected from C₂H₄, C₃H₆, or C₄H₈, and combinations thereof; m is an integer ranging from about 1 to about 5; and n is an integer ranging from about 1 to about 50 in one aspect, from about 3 to about 35 in another aspect, from about 5 to about 25 in a still another aspect, and from about 8 to about 20 in a further aspect; subject to the proviso that R⁹⁷ cannot both be methyl at the same time.

Exemplary nonionic dimethicone copolyols containing a terminal polyoxyalkylene moietie(s) also are commercially available under the Silsoft® trade name from Momentive Performance Materials under product designations 810 (PEG-8 Dimethicone), 860 (PEG-10 Dimethicone), 870 (PEG-12 Dimethicone), and 900 (PPG-12 Dimethicone).

In one aspect, the nonionic dimethicone copolyol contains esterified pendant polyoxyalkylene moieties and can be represented by the formula:

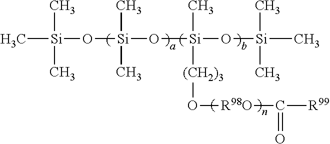

wherein a represents an integer ranging from about 0 or 1 to about 500; b is an integer ranging from about 1 to about 100; (R⁹⁸O)ₙ is a polyoxyalkylene moiety which can be arranged as a homopolymer, a random copolymer, or a block copolymer of oxyalkylene units; R⁹⁸ is a divalent alkylene moiety selected from C₂H₄, C₃H₆, or C₄H₈, and combinations thereof; and n is an integer ranging from about 1 to about 50 in one aspect, from about 3 to about 35 in another aspect, from about 5 to about 25 in a still another aspect, and from about 8 to about 20 in a further aspect; and R⁹⁹ is C₁ to C₂₁ alkyl. In one aspect the acyl radical —C(O)R⁹⁹ that terminates the polyoxyalkylene moiety is derived from a saturated or unsaturated carboxylic or fatty acid obtained from natural or synthetic sources. These acids can be linear or branched. Suitable acids are selected from, but are not limited to caproic acid, enanthic acid, caprylic acid, pelargonic acid, capric acid, undecanoic acid lauric acid, myristic acid, palmitic acid, stearic acid, isosteric acid, oleic acid, linoleic acid, ricinoleic acid, and behenic acid which are typically obtained by hydrolyzing vegetable oils and animal oils such as coconuts oils, palm oil, tallow, linseed oil and soybean oil.

Dimethicone copolyol esters and methods for their preparation are disclosed in U.S. Pat. No. 5,136,063, which is herein incorporated by reference. Exemplary dimethicone copolyol esters are commercially available under the Silsence™ trade name as product designations SW-12 (Dimethicone PEG-7 Cocoate) and DW-18 (Dimethicone PEG-7 isosterate) from Lubrizol Advanced Materials, Inc.

Other useful dimethicone copolyol esters contain at least one terminal polyoxyalkylene ester moiety as described in U.S. Pat. No. 5,180,843, which is herein incorporated by reference.

In one aspect, the dimethicone copolyol contains a quaternium moiety and can be represented by the formula:

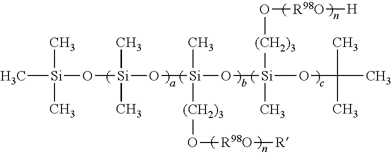

wherein a represents an integer ranging from about 0 or 1 to about 200; b is an integer ranging from about 1 to about 100; c is an integer ranging from about 0 or 1 to about 200; (R⁹⁸O)ₙ is a polyoxyalkylene moiety which can be arranged as a homopolymer, a random copolymer, or a block copolymer of oxyalkylene units; R⁹⁸ is a divalent alkylene moiety selected from C₂H₄, C₃H₆, or C₄H₈, and combinations thereof; and n independently is an integer ranging from about 1 to about 50 in one aspect, from about 3 to about 35 in another aspect, from about 5 to about 25 in a still another aspect, and from about 8 to about 20 in a further aspect; R′ is selected from a radical of the following formulas:

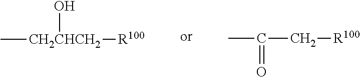

wherein R¹⁰⁰ is a quaternary nitrogen containing moiety selected from the formulas:

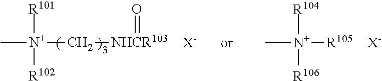

wherein R¹⁰¹ and R¹⁰² independently are selected from methyl or ethyl; R¹⁰³ is a C₅ to C₂₁ alkyl group; R¹⁰⁴, R¹⁰⁵, R¹⁰⁶ independently represent C₁ to C₂₀ alkyl; and X⁻ is a salt forming anion. In one aspect, R¹⁰¹ and R¹⁰² are both methyl or both ethyl and R¹⁰³ is a C₁₁ to C₂₁ alkyl group. In one aspect, two of R¹⁰⁴, R¹⁰⁵ or R¹⁰⁶ are methyl and the remaining R¹⁰⁴, R¹⁰⁵ or R¹⁰⁶ that is not methyl is selected from a C₁₂ to C₂₀ alkyl group. In one aspect, X⁻ is a chloride anion.

Dimethicone copolyol quaternary nitrogen containing compounds are disclosed in U.S. Pat. Nos. 5,098,979 and 5,166,297, the disclosures of which are herein incorporated by reference. A suitable commercially available dimethicone copolyol quaternary compound (Silicone Quaternium-8) is available under the Silsence™ Q-Plus trade name from Lubrizol Advanced Materials, Inc.

Other exemplary quaternary nitrogen containing silicones that are useful in the disclosed technology Are Quaternium-80, Silicone Quaternium-1, Silicone Quaternium-2, Silicone Quaternium-2 Panthenol Succinate, Silicone Quaternium-3, Silicone Quaternium-4, Silicone Quaternium-5, Silicone Quaternium-6, Silicone Quaternium-7, Silicone Quaternium-9, Silicone Quaternium-10, Silicone Quaternium-11, Silicone Quaternium-12, Silicone Quaternium-15, Silicone Quaternium-16, Silicone Quaternium-16/Glycidoxy Dimethicone Crosspolymer, Silicone Quaternium-17, Silicone Quaternium-18, Silicone Quaternium-20 and Silicone Quaternium-21.

In one aspect, the dimethicone copolyol contains an amine functional group. Amine functional dimethicone copolyols can be represented by the formula:

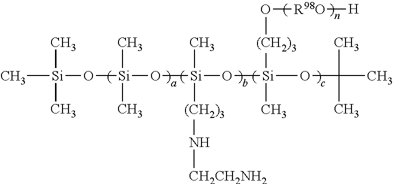

wherein a represents an integer ranging from about 0 or 1 to about 200; b is an integer ranging from about 1 to about 100; c is an integer ranging from about 1 to about 200; (R⁹⁸O)ₙ is a polyoxyalkylene moiety which can be arranged as a homopolymer, a random copolymer, or a block copolymer of oxyalkylene units; R⁹⁸ is a divalent alkylene moiety selected from C₂H₄, C₃H₆, or C₄H₈, and combinations thereof; and n independently is an integer ranging from about 1 to about 50 in one aspect, from about 3 to about 35 in another aspect, from about 5 to about 25 in a still another aspect, and from about 8 to about 20 in a further aspect. A commercially available amine functional dimethicone copolyol is marketed under the trade name Silsense™ A-21 silicone (PEG-7 Amodimethicone) by Lubrizol Advanced Materials, Inc.

In one aspect, the dimethicone copolyol contains phosphate ester functionality. These compounds can be represented by the formula:

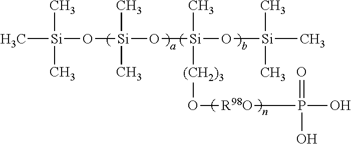

wherein a represents an integer ranging from about 0 or 1 to about 500; b is an integer ranging from about 1 to about 100; (R⁹⁸O)ₙ is a polyoxyalkylene moiety which can be arranged as a homopolymer, a random copolymer, or a block copolymer of oxyalkylene units; R⁹⁸ is a divalent alkylene moiety selected from C₂H₄, C₃H₆, or C₄H₈, and combinations thereof; and n is an integer ranging from about 1 to about 50 in one aspect, from about 3 to about 35 in another aspect, from about 5 to about 25 in a still another aspect, and from about 8 to about 20 in a further aspect. A commercially available amine functional dimethicone copolyol is marketed under the trade name Silsense PE-100 silicone (Dimethicone PEG-8 Phosphate) by Lubrizol Advanced Materials, Inc.

### B. Organic Oils

The conditioning component of the compositions of the disclosed technology optionally can also contain hydrocarbon oil conditioners. Suitable conditioning oils for use as conditioning agents in the compositions of the disclosed technology include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils typically contain about 12 to 19 carbon atoms. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2,2,4,4,6,6,8,8-dimethyl-10-methylundecane and 2,2,4,4,6,6-dimethyl-8-methylnonane, available from Permethyl Corporation. Hydrocarbon polymers such as polybutene and polydecene. A preferred hydrocarbon polymer is polybutene, such as the copolymer of isobutylene and butene. A commercially available material of this type is L-14 polybutene from BP Chemical Company.

Liquid polyolefin conditioning oils can be used in the hair conditioning compositions of the present technology. The liquid polyolefin conditioning agents are typically poly-α-olefins that have been hydrogenated. Polyolefins for use herein can be prepared by the polymerization of C₄ to about C₁₄ olefinic monomers. Non-limiting examples of olefinic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. In one aspect of the disclosed technology, hydrogenated α-olefin monomers include, but are not limited to: 1-hexene to 1-hexadecenes, 1-octene to 1-tetradecene, and mixtures thereof.

Fluorinated or perfluorinated oils are also contemplated within the scope of the present technology. Fluorinated oils include perfluoropolyethers described in European Patent 0 486 135 and the fluorohydrocarbon compounds described in WO 93/11103. The fluorinated oils may also be fluorocarbons such as fluoramines, e.g., perfluorotributylamine, fluorinated hydrocarbons, such as perfluorodecahydronaphthalene, fluoroesters, and fluoroethers.

Natural oil conditioners are also useful in the practice of the disclosed technology and include but are not limited to peanut, sesame, avocado, coconut, cocoa butter, almond, safflower, corn, cotton seed, sesame seed, walnut oil, castor, olive, jojoba, palm, palm kernel, soybean, wheat germ, linseed, sunflower seed; eucalyptus, lavender, vetiver, litsea, cubeba, lemon, sandalwood, rosemary, chamomile, savory, nutmeg, cinnamon, hyssop, caraway, orange, geranium, cade, and bergamot oils, fish oils, glycerol tricaprocaprylate; and mixtures thereof.

Ester oil conditioners include, but are not limited to, fatty esters having at least 10 carbon atoms. These fatty esters include esters derived from fatty acids or alcohols (e.g., mono-esters, polyhydric alcohol esters, and di- and tri-carboxylic acid esters). The fatty esters hereof may include or have covalently bonded thereto other compatible functionalities, such as amides and alkoxy moieties (e.g., ethoxy or ether linkages, etc.).

Exemplary fatty esters include, but are not limited to isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the compositions of the disclosed technology are mono-carboxylic acid esters of the general formula R⁶⁰C(O)OR⁶¹, wherein R⁶⁰ and R⁶¹ are alkyl or alkenyl radicals, and the sum of carbon atoms in R⁶⁰ and R⁶¹ is at least 10 in one aspect, and at least 22 in another aspect of the disclosed technology.

Still other fatty esters suitable for use in the compositions of the disclosed technology are di- and tri-alkyl and alkenyl esters of carboxylic acids, such as esters of C₄ to C₈ dicarboxylic acids (e.g., C₁ to C₂₂ esters, preferably C₁ to C₆, of succinic acid, glutaric acid, adipic acid). Specific non-limiting examples of di- and tri-alkyl and alkenyl esters of carboxylic acids include isocetyl stearyol stearate, diisopropyl adipate, and tristearyl citrate.

Other fatty esters suitable for use in the compositions of the disclosed technology are those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Specific non-limiting examples of suitable synthetic fatty esters include: P-43 (C₈ to C₁₀ triester of trimethylolpropane), MCP-684 (tetraester of 3,3 diethanol-1,5 pentadiol), MCP 121 (C₈ to C₁₀ diester of adipic acid), all of which are available from ExxonMobil Chemical Company.

The amount of organic oil conditioning agents can range from about 0.05 to about 10 wt. %, in one aspect, from about 0.5 to about 5 wt. % in another aspect, and from about 1 to about 3 wt. % in a further aspect, based on the total weight of the composition.

### C. Cationic Compounds and Polymers

Cationic Compounds are non-polymeric compounds that contain at least one cationic moiety or at least one moiety that can be ionized to form a cationic moiety. Cationic polymers are polymers that contain at least one repeating unit that contains at least one cationic moiety or at least one moiety that can be ionized to form a cationic moiety. Typically these cationic moieties are nitrogen containing groups such as quaternary ammonium salts or protonated amino groups. The cationic protonated amines can be primary, secondary, or tertiary amines. In one aspect, the cationic conditioning agents include quaternary nitrogen containing non-polymeric and polymeric materials that well known in the art for hair conditioning.

In one aspect, the auxiliary conditioning agent different than (a) is a dialkyl quaternary ammonium compound corresponding to the general formula: (R⁷⁵)(R⁷⁶)(R⁷⁷)(R⁷⁸)N⁺CA⁻ wherein two of R⁷⁵, R⁷⁶, R⁷⁷ and R⁷⁸ are selected from an alkyl group containing from 12 to 22 carbon atoms or an aromatic, alkoxy, polyoxyalkylene, alkylamido, hydroxyalkyl, aryl or alkylaryl group having up to about 30 carbon atoms with or without an ester group; and the remainder of R⁷⁵, R⁷⁶, R⁷⁷ and R⁷⁸ are independently selected from an alkyl group containing from 1 to about 4 carbon atoms or an alkoxy, polyoxyalkylene, alkylamido, hydroxyalkyl, aryl or alkylaryl group having up to about 4 carbon atoms; and CA⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate (e.g., methosulfate and ethosulfate) moieties. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated or branched. In one embodiment, two of R⁷⁵, R⁷⁶, R⁷⁷ and R⁷⁸ are selected from an alkyl group containing from 12 to 22 carbon atoms in one aspect, from 14 to 20 carbon atoms in another aspect, and from 16 to 18 carbon atoms in a further aspect; the remainder of R⁷⁵, R⁷⁶, R⁷⁷ and R⁷⁸ are independently selected from CH₃, C₂H₅, C₂H₄OH, and mixtures thereof. Any two of R⁷⁵, R⁷⁶, R⁷⁷, and R⁷⁸ together with the nitrogen atom to which they are attached can be taken together to form a ring structure containing 5 to 6 carbon atoms, one of said carbon atoms can optionally be replaced with a heteroatom selected from nitrogen, oxygen or sulfur. CA⁻ is a salt-forming anion selected from halogen, (e.g. chloride, bromide), acetate, citrate, lactate, glycolate, phosphate, nitrate, sulfate, and alkylsulfate (e.g., methosulfate, ethosulfate).

Non-limiting examples of dialkyl quaternized ammonium compounds include dicocodimonium chloride; dicocodimonium bromide; dimyristyldimonium chloride; dimyristyldimonium bromide; dicetyldimonium chloride; dicetyldimonium bromide; dicetylmethylbenzylmonium chloride; distearyldimonium chloride; distearyldimonium bromide; dimetyldi(hydrogenated tallow)monium chloride; hydroxypropylbisstearylmonium chloride; distearylmethylbenzylmonium chloride; dibehenyl/diarachidyldimonium chloride; dibehenyl/diarachidyldimonium bromide; dibehenyldimonium chloride; dibehenyldimonium bromide; dibehenyldimonium methosulfate; dibehenylmethylbenzylmonium chloride; dihydrogenated tallow benzylmonium chloride; dihydrogenated tallowethyl hydroxyethylmonium methosulfate; dihydrogenated tallow hydroxyethylmonium methosulfate; di-C₁₂-C₁₅ alkyldimonium chloride; di-C₁₂-C₁₈ alkyldimonium chloride; di-C₁₄-C₁₈ alkyldimonium chloride; dicocoylethyl hydroxyethylmonium methosulfate; disoyoylethyl hydroxyethylmonium methosulfate; dipalmitoylethyldimonium chloride; dihydrogenated palmoylethyl hydroxyethylmonium methosulfate; dihydrogenated tallowamidoethyl hydroxyethylmonium chloride; dihydrogenated tallowamidoethyl hydroxyethylmonium methosulfate; dihydrogenated tallowoylethyl hydroxyethylmonium methosulfate; distearoylethyl hydroxyethylmonium methosulfate; and Quaternium-82.

In one aspect, the auxiliary conditioning agent different than (a) is an asymmetric dialkyl quaternary ammonium compound corresponding to the general formula: (R⁸⁰)(R⁸¹)(R⁸²)(R⁸³)N⁺CA⁻ wherein R⁸⁰ is selected from an alkyl group containing from 12 to 22 carbon atoms or an aromatic, alkoxy, polyoxyalkylene, alkylamido, hydroxyalkyl, aryl or alkylaryl group containing up to about 22 carbon atoms; R⁸¹ is selected from an alkyl group containing from 5 to 12 carbon atoms or an aromatic, alkoxy, polyoxyalkylene, alkylamido, hydroxyalkyl, aryl or alkylaryl group containing up to about 12 carbon atoms; R⁸² and R⁸³ are independently selected from an alkyl group containing from 1 to about 4 carbon atoms or an aromatic, alkoxy, polyoxyalkylene, alkylamido, hydroxyalkyl, aryl or alkylaryl group containing up to about 4 carbon atoms; and CA⁻ is a salt-forming anion such as, for example, halogen, (e.g. chloride, bromide), acetate, citrate, lactate, glycolate, phosphate, nitrate, sulfate, and alkylsulfate (e.g., methosulfate, ethosulfate). The alkyl groups can contain, in addition to carbon and hydrogen atoms, ether linkages, ester linkages, and other moieties such as amino groups. The longer chain alkyl groups, e.g., those of about 12 carbons, or higher, can be saturated or unsaturated and/or straight or branched. In one embodiment, R⁸⁰ is selected from a non-functionalized alkyl group containing from 12 to 22 carbon atoms in one aspect, from 14 to 20 carbon atoms in another aspect, and from 16 to 18 carbon atoms in a further aspect; R⁸¹ is selected from a non-functionalized alkyl group containing from 5 to 12 carbon atoms in one aspect, from 6 to 10 carbon atoms in another aspect, and 8 carbon atoms in a further aspect; R⁸² and R⁸³ are independently selected from CH₃, C₂H₅, C₂H₄OH, and mixtures thereof; and CA⁻ is selected from Cl, Br, CH₃OSO₃, C₂H₅OSO₃, and mixtures thereof. In one aspect, R⁸⁰ is a straight, saturated non-functionalized alkyl group, and R⁸¹ is a branched, saturated non-functionalized alkyl group. In one aspect, the branched group of R⁸¹ is a straight, saturated alkyl group containing from 1 to 4 carbon atoms, and in another aspect, R⁸¹ is an alkyl group containing 2 carbon atoms.

Non-limiting examples of asymmetric dialkyl quaternized ammonium salt compounds include: stearylethylhexyldimonium chloride, stearylethylhexyldimonium bromide; stearyl ethylhexyl dimonium methosulfate; cetearyl ethylhexyldimonium methosulfate.

A number of quaternary nitrogen containing compounds their manufacturers and general descriptions of their chemical characteristics are found in the CTFA Dictionary and in the International Cosmetic Ingredient Dictionary, Vol. 1 and 2, 5th Ed., published by the Cosmetic Toiletry and Fragrance Association, Inc. (CTFA) (1993), the pertinent disclosures of which are incorporated herein by reference. The name assigned to the ingredients by the CTFA or by the manufacturer is used for convenience.

Other non-limiting examples of quaternary ammonium compounds useful as auxiliary conditioning agents different than (a) include acetamidopropyl trimonium chloride, behenamidopropyl ethyldimonium ethosulfate, cetethyl morpholinium ethosulfate, cocoamidopropyl ethyldimonium ethosulfate, dicetyldimonium chloride, hydroxyethyl behenamidopropyl dimonium chloride, Quaternium-18, Quaternium-26, Quaternium-27, Quaternium-53, Quaternium-63, Quaternium-70, Quaternium-72, Quaternium-76 PPG-9 diethylmonium chloride, PPG-25 diethylmonium chloride, PPG-40 stearalkonium chloride, isostearamidopropyl ethyldimonium ethosulfate, and mixtures thereof.

In one aspect, the auxiliary conditioning agent different than (a) is a quaternary nitrogen containing ether substituted, ethoxylated alkyl glucoside compound represented by the formula:

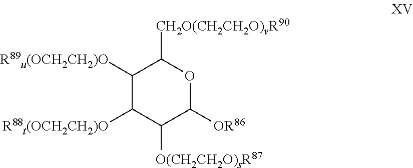

wherein R⁸⁶ represents C₁ to C₅ alkyl, e.g., methyl, ethyl, propyl; R⁸⁷, R⁸⁸, R⁸⁹ and R⁹⁰ independently represent hydrogen; a C₁ to C₂₂ alkyl group; a C₂ to C₂₂ alkenyl group; an acyl substituent represented by —C(O)R⁹⁵, where R⁹⁵ is selected from C₅ to C₂₁ alkyl or C₅ to C₂₁ alkenyl; and where at least one of R⁸⁷, R⁸⁸, R⁸⁹ and R⁹⁰ represents a quaternary nitrogen moiety represented by the formula:

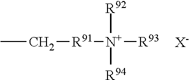

wherein R⁹¹ is a C₁ to C₅ alkylene, e.g., methylene, ethylene, propylene, or a C₁ to C₅ hydroxy substituted alkylene, e.g., hydroxyemethylene, hydroxyethylene, hydroxypropylene; and R⁹², R⁹³, and R⁹⁴ independently represent C₁ to C₂₂ alkyl, e.g., methyl, ethyl, propyl, butyl, decyl, dodecyl, hexadecyl, octadecyl, behenyl; C₆ to C₁₀ aryl, e.g, phenyl, tolyl, benzyl; and X⁻ is a salt forming anion, such as, for example, halogen, (e.g. chloride, bromide), acetate, citrate, lactate, glycolate, phosphate, nitrate, sulfate, and alkylsulfate (e.g., methosulfate, ethosulfate); and wherein the sum of s+t+u+v ranges from about 1 to about 200 in one aspect, from about 5 to about 100 in another aspect, from about 8 to about 50 in a further aspect, and from about 10 to about 25 in a still further aspect.

In one aspect, R⁸⁶ is a methyl group; at least one of the R⁸⁷ to R⁹⁰ substituents is a quaternary nitrogen containing moiety, with the remaining R⁸⁷ to R⁹⁰ substituent(s) not substituted with the quaternary nitrogen moiety being selected from hydrogen; R⁹¹ is hydroxyalkylene, with two of R⁹² to R⁹⁴ representing methyl and the remaining substituent of R⁹² to R⁹⁴ that is not methyl is selected from a C₁₀ to C₂₂ alkyl or C₁₀ to C₂₂ alkenyl group.

The quaternary nitrogen containing ether substituted, ethoxylated alkyl glucoside compounds set forth under formula XV above are disclosed in U.S. Pat. No. 5,138,043, which is herein incorporated by reference. In one aspect, a suitable quaternary nitrogen containing ether substituted, ethoxylated alkyl glucoside compound is Lauryl Methyl Gluceth-10 Hydroxypropyldimonium Chloride which is commercially available under the Gluquat™ 125 trade name from Lubrizol Advanced Materials, Inc.

Cationic polymers are also useful as auxiliary conditioning agents alone or in combination with the other auxiliary conditioning agents described herein. Suitable cationic polymers can be synthetically derived or natural polymers can be synthetically modified to contain cationic moieties. A number of cationic moiety containing polymers their manufacturers and general descriptions of their chemical characteristics are found in the CTFA Dictionary and in the International Cosmetic Ingredient Dictionary, Vol. 1 and 2, 5th Ed., published by the Cosmetic Toiletry and Fragrance Association, Inc. (CTFA) (1993), the pertinent disclosures of which are incorporated herein by reference.

In one aspect, the cationic polymer contains at least one repeating unit containing a quaternary ammonium salt moiety. Such polymers can be prepared by the polymerization of a diallylamine such as dialkyldiallylammonium salt or copolymer thereof in which the alkyl group contains 1 to about 22 carbon atoms in one aspect and methyl or ethyl in another aspect. Copolymers containing a quaternary moiety derived from a dialkyldiallylammonium salt and an anionic component derived from anionic monomers of acrylic acid and methacrylic acid are suitable conditioning agents. Also suitable are, polyampholyte terpolymers having a cationic component prepared from a derivative of diallylamine, such as a dimethyldiallylammonium salt, an anionic component derived from anionic monomers of acrylic acid or 2-acrylamido-2-methylpropane sulfonic acid and a nonionic component derived from nonionic monomers of acrylamide. The preparation of such quaternary ammonium salt moiety containing polymers can be found, for example, in U.S. Pat. Nos. 3,288,770; 3,412,019; 4,772,462 and 5,275,809, the pertinent disclosures of which are incorporated herein by reference.

In one aspect, suitable cationic polymers include the chloride salts of the foregoing quaternized homopolymers and copolymers in which the alkyl group is methyl or ethyl, and are commercially available under the Merquat® series of trademarks from Lubrizol Advanced Materials, Inc.

A homopolymer prepared from diallyl dimethyl ammonium chloride (DADMAC) having the CTFA name, Polyquaternium-6, is available under the Merquat 100 and Merquat 106 trademark. A copolymer prepared from DADMAC and acrylamide having the CTFA name, Polyquaternium-7, is sold under the Merquat 550 trademark. Another copolymer prepared from DADMAC and acrylic acid having the CTFA name, Polyquaternium-22, is sold under the Merquat 280 trademark. The preparation of Polyquaternium-22 and its related polymers is described in U.S. Pat. No. 4,772,462, the pertinent disclosures of which are incorporated herein by reference.

Also useful is an ampholytic terpolymer prepared from a nonionic component derived from acrylamide or methyl acrylate, a cationic component derived from DADMAC or methacrylamidopropyl trimethyl ammonium chloride (MAPTAC), and an anionic component derived from acrylic acid or 2-acrylamido-2-methylpropane sulfonic acid or combinations of acrylic acid and 2-acrylamido-2-methylpropane sulfonic acid. An ampholytic terpolymer prepared from acrylic acid, DADMAC and acrylamide having the CTFA name, Polyquarternium-39, is available under the Merquat Plus 3330 trademark. Another ampholytic terpolymer prepared from acrylic acid, methacrylamidopropyl trimethyl ammonium chloride (MAPTAC) and methyl acrylate having the CTFA name, Polyquarternium-47, is available under the Merquat 2001 trademark. Still another ampholytic terpolymer prepared from acrylic acid, MAPTAC and acrylamide having the CTFA name, Polyquarternium-53, is available under the Merquat 2003PR trademark. The preparation of such terpolymers is described in U.S. Pat. No. 5,275,809, the pertinent disclosures of which are incorporated herein by reference.

Other cationic polymers and copolymers suitable as conditioners in the disclosed technology have the CTFA names Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquarternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-42, Polyquaternium-43, Polyquaternium-44, Polyquaternium-45, Polyquaternium-46, Polyquaternium-47, Polyquaternium-48, Polyquaternium-49, Polyquaternium-50, Polyquaternium-51, Polyquaternium-52, Polyquaternium-53, Polyquaternium-54, Polyquarternium-55, Polyquaternium-56, Polyquaternium-57, Polyquaternium-58, Polyquaternium-59, Polyquaternium-60, Polyquaternium-61, Polyquaternium-62, Polyquaternium-63, Polyquaternium-64, Polyquaternium-65, Polyquaternium-66, Polyquaternium-67, Polyquaternium-68, Polyquaternium-69, Polyquaternium-70, Polyquaternium-71, Polyquaternium-72, Polyquaternium-73, Polyquaternium-74, Polyquaternium-75, Polyquaternium-76, Polyquaternium-77, Polyquaternium-78, Polyquaternium-79, Polyquaternium-80, Polyquaternium-81, Polyquaternium-82, Polyquaternium-83, Polyquaternium-84, Polyquaternium-85, Polyquaternium-86, Polyquaternium-87, and mixtures thereof.

Exemplary cationically modified natural polymers suitable for use in the hair conditioning composition includes polysaccharide polymers, such as cationically modified cellulose and cationically modified starch derivatives modified with a quaternary ammonium halide moiety. Exemplary cationically modified cellulose polymers are salts of hydroxyethyl cellulose reacted with trimethyl ammonium substituted epoxide (CTFA, Polyquaternium-10). Other suitable types of cationically modified cellulose include the polymeric quaternary ammonium salts of hydroxyethyl cellulose reacted with lauryl dimethyl ammonium substituted epoxide (CTFA, Polyquaternium-24). Cationically modified potato starch having the CTFA name, Starch Hydroxypropyltrimonium Chloride, is available under the Sensomer™ CI-50 trademark, from Lubrizol Advanced Materials, Inc.

Other suitable cationically modified natural polymers include cationic polygalactomannan derivatives such as guar gum derivatives and cassia gum derivatives, e.g., CTFA: Guar Hydroxypropyltrimonium Chloride, Hydroxypropyl Guar Hydroxypropyltrimonium Chloride, and Cassia Hydroxypropyltrimonium Chloride. Guar hydroxypropyltrimonium chloride is commercially available under the Jaguar™ trade name series from Rhodia Inc. and the N-Hance trade name series from Ashland Inc. Cassia Hydroxypropyltrimonium Chloride is commercially available under the Sensomer™ CT-250 and Sensomer™ CT-400 trademarks from Lubrizol Advanced Materials, Inc.

The non-polymeric cationic compounds and cationic polymers can be present from about 0.05 to about 5 wt. % percent in one aspect, from about 0.1 to about 3 wt. percent in another aspect, and from about 0.5 to about 2.0 wt. % in a further aspect (based on the total weight of the composition).

### Insoluble and Particulate Materials

The nonionic, amphiphilic polymers of the disclosed technology can stably suspend insoluble and particulate materials in the conditioning compositions of the disclosed technology. Examples of such optional materials include opacifiers and pearlescent agents (e.g., mica, coated mica, ethylene glycol monostearate (EGMS), ethylene glycol distearate (EGDS), polyethylene glycol monostearate (PGMS) or polyethyleneglycol distearate (PGDS)) and aesthetic and encapsulated materials (e.g., gas bubbles, liposomes, microsponges, cosmetic beads, cosmetic microcapsules, and flakes.

### Auxiliary Viscosity Modifier

The conditioning compositions of the disclosed technology must be easily pourable with a shear thinning index of less than 0.5 at shear rates between 0.1 and 1 reciprocal second, and an optical transmission of at least 10%. If desired the nonionic, amphiphilic polymers of the disclosed technology can be utilized in combination with an auxiliary rheology modifier (thickener). In one aspect, the nonionic, amphiphilic, emulsion polymer of the disclosed technology can be combined with a nonionic rheology modifier to enhance the yield stress value of a composition in which it is included. Any rheology modifier is suitable, so long as such is soluble in water, stable and contains no ionic or ionizable groups. Suitable rheology modifiers include, but are not limited to natural gums (e.g., polygalactomannan gums selected from fenugreek, cassia, locust bean, tara and guar), modified cellulose (e.g., ethylhexylethylcellulose (EHEC), hydroxybutylmethylcellulose (HBMC), hydroxyethylmethylcellulose (HEMC), hydroxypropylmethylcellulose (HPMC), methyl cellulose (MC), hydroxyethylcellulose (HEC), hydroxypropylcellulose (HPC) and cetyl hydroxyethylcellulose); methylcellulose and mixtures thereof; polyethylene glycols (e.g., PEG 4000, PEG 6000, PEG 8000, PEG 10000, PEG 20000), polyvinyl alcohol, polyacrylamides (homopolymers and copolymers), and hydrophobically modified ethoxylated urethanes (HEUR). The rheology modifier can be utilized in an amount ranging from about 0.5 to about 25 wt. % in one aspect, from about 1 to about 15 wt. % in another aspect, and from about 2 to about 10 wt. % in a further aspect, based on the weight of the total weight of the composition.

In one embodiment, the hair care composition of the disclosed technology is a moderately viscous mixture, having a Brookfield viscosity in the range of from about 1000 mPa·s to about 15,000 mPa·s in one aspect, from about 2,000 mPa·s to about 10,000 mPa·s in another aspect, from about 3,500 mPa·s to about 8,500 mPa·s in still another aspect, and from about 4,500 mPa·s to about 5500 mPa·s in a further aspect. The viscosities are adjustable by changing the amount of nonionic, amphiphilic polymeric material in the hair care composition. The product should be pourable from a relatively narrow mouth bottle (approximately 1.5 cm in diameter) and the product will not be so thin to run off of the hands or the hair.

The hair conditioning compositions of the present technology are stable indefinitely at temperatures normally found in commercial product storage and shipping. The compositions resist phase separation or settling of composition ingredients at a temperature of about 20° C. to about 25° C. essentially indefinitely. The compositions also must demonstrate sufficient stability to phase separation and settling of ingredients at temperatures normally found in commercial product storage and shipping to remain unaffected for periods of one year or more.

### Botanicals

The hair conditioning compositions of the disclosed technology can contain one or more botanical agents. Suitable botanical agents can include, for example, extracts from Echinacea (e.g., sp. angustifolia, purpurea, pallida), yucca glauca, willow herb, basil leaves, Turkish oregano, carrot root, grapefruit, fennel seed, rosemary, tumeric, thyme, blueberry, bell pepper, blackberry, spirulina, black currant fruit, tea leaves, such as for, example, Chinese tea, black tea (e.g., var. Flowery Orange Pekoe, Golden Flowery Orange Pekoe, Fine Tippy Golden Flowery Orange Pekoe), green tea (e.g., var. Japanese, Green Darjeeling), oolong tea, coffee seed, dandelion root, date palm fruit, gingko leaf, green tea, hawthorn berry, licorice, apricot kernel, sage, strawberry, sweet pea, tomato, sunflower seed extract, sandalwood extract, grape seed, aloe leaf, vanilla fruit, comfrey, arnica, Centella asiatica, cornflower, horse chestnut, ivy, Macadamia ternifolia seed, magnolia, oat, pansy, skullcap, seabuckthorn, white nettle, and witch hazel. Botanical extracts may also include, for example, chlorogenic acid, glutathione, glycrrhizin, neohesperidin, quercetin, rutin, morin, myricetin, absinthe, and chamomile.

In one aspect, the hair care composition can contain from about 0.01 wt. % to about 10 wt. % of one or more of the botanical extracts set forth above, from about 0.05 wt. % to about to about 5 wt. % in another aspect, from about 0.1 wt. % to about 3 wt. % in still another aspect, and from about 0.5 wt. % to about 1 wt. % in a further aspect, based on the total weight of the composition.

### Amino Acids

The hair conditioning composition provided herein can contain one or more amino acids. Examples of amino acids that can be used include, without limitation, capryl keratin amino acids, capryl silk amino acids, jojoba amino acids, keratin amino acids, palmitoyl keratin amino acids, palmitoyl silk amino acids, sodium cocoyl amino acids, sodium cocoyl silk amino acids, and sweet almond amino acids.

The hair conditioning composition can include an appropriate amount of amino acid(s). The amount of amino acid ranges from about 0.001 wt. % to about 5 wt. % in one aspect, from about 0.01 wt. % percent to about 3 wt. % in another aspect, from about 0.1 wt. % to about 2 wt. % in still another aspect, and from about 0.5 wt. % to about 1 wt. % in a further aspect, based on the total weight of the composition.

### Vitamins

The hair conditioning composition can contain one or more vitamins. Examples of vitamins that can be used include, without limitation, niacinamide, sodium starch octenylsuccinate, calcium pantothenate, maltodextrin, sodium ascorbyl phosphate, tocopheryl acetate, pyridoxine HCl, silica, panthenol (e.g., Pro Vitamin B5), phytantriol, calcium pantothenate (e.g., vitamin B5), vitamin E, and vitamin E esters (e.g., tocopheryl acetate, tocopheryl nocotinate, tocopheryl palmitate, or tocopheryl retinoate). The amount of vitamin(s) can range from about 0.05 wt. % to about 10 wt. % in one aspect, from about 0.1 wt. % to about 5 wt. % in another aspect, from about 0.5 wt. % to about 3 wt. % in still another aspect, and from about 0.75 wt. % to about 1 wt. % in a further aspect, based on the total weight of the composition.

### Chelating Agents

Chelating agents can be employed to stabilize the composition against the deleterious effects of metal ions. When utilized, suitable chelating agents include EDTA (ethylene diamine tetraacetic acid) and salts thereof such as disodium EDTA, citric acid and salts thereof, cyclodextrins, and the like, and mixtures thereof.

Such suitable chelating agents can comprise 0.001 wt. % to 3 wt. %, such as 0.01 wt. % to 2 wt. %, or 0.01 wt. % to 1 wt. % of the total weight of the hair conditioning composition.

### Buffering Agents

Buffering agents can be used in the conditioning compositions. Suitable buffering agents include alkali or alkali earth metal carbonates, phosphates, bicarbonates, citrates, borates, acetates, acid anhydrides, succinates, and the like, such as sodium phosphate, sodium citrate, sodium acetate, sodium bicarbonate, and sodium carbonate.

### pH Adjusting Agents

In one aspect, the pH of the hair conditioning composition of the disclosed technology ranges from about 2.5 to about 5.5. To provide the desired pH, the composition may be adjusted with one or more pH modifiers selected from organic and inorganic acids and bases. The pH of the composition can be adjusted with any combination of acidic and/or basic pH adjusting agents known to the art. Acidic materials include organic acids and inorganic acids, in particular, monocarboxylic acids, dicarboxylic acids, and tricarboxylic acids, for example, acetic acid, citric acid, tartaric acid, alpha-hydroxy acids, beta-hydroxy acids, salicylic acid, lactic acid, malic acid, glycolic acid, amino acids, and natural fruit acids, or inorganic acids, for example, hydrochloric acid, nitric acid, sulfuric acid, sulfamic acid, phosphoric acid, and combinations thereof.

Basic materials include inorganic and organic bases, and combinations thereof. Examples of inorganic bases include but are not limited to the alkali metal hydroxides (e.g., potassium hydroxide, sodium hydroxide) and alkali metal carbonates (e.g., potassium carbonate, sodium carbonate), and alkali metal salts such as sodium borate (borax), sodium phosphate, sodium pyrophosphate, and the like; and mixtures thereof. Examples of organic bases include ammonium hydroxide, triethanolamine (TEA), diisopropanolamine, triisopropanolamine, aminomethyl propanol, dodecylamine, cocamine, oleamine, morpholine, triamylamine, triethylamine, tetrakis(hydroxypropyl)ethylenediamine, L-arginine, aminomethyl propanol, tromethamine (2-amino 2-hydroxymethyl-1,3-propanediol), and PEG-15 cocamine.

The pH adjusting agent(s) and/or buffering agent is utilized in any amount necessary to obtain and/or maintain a desired pH value in the composition. The pH of the conditioning compositions of the disclosed technology can be any desired value so long as it does not deleteriously affect the components and/or the efficacy of the composition. In one aspect, the pH can range from about 2.5 to about 7. In another aspect, the pH can range from about 3.5 to about 6.5, and in a further aspect from about 4.5 to about 5.5.

### Preservatives

In one aspect, any preservative suitable for use in personal care formulations can be used in the hair conditioning compositions of the present technology. Suitable preservatives include polymethoxy bicyclic oxazolidine, methyl paraben, propyl paraben, ethyl paraben, butyl paraben, benzyltriazole, DMDM hydantoin (also known as 1,3-dimethyl-5,5-dimethyl hydantoin), imidazolidinyl urea, phenoxyethanol, phenoxyethylparaben, methylisothiazolinone, methylchloroisothiazolinone, benzoisothiazolinone, triclosan, and suitable polyquaternium compounds as disclosed above (e.g., Polyquaternium-1).

In another aspect, acid based preservatives are useful in the exemplary compositions. The use of acid based preservatives facilitates the formulation of products in the low pH range. Lowering the pH of a formulation inherently provides an inhospitable environment for microbial growth. Moreover, formulating at low pH enhances the efficacy of acid based preservatives, and affords a personal care product which maintains an acidic pH balance on the skin. Any acid based preservative that is useful in personal care products can be used in the exemplary compositions. In one aspect the acid preservative is a carboxylic acid compound represented by the formula: R⁸⁵C(O)OH, wherein R⁸⁵ represents hydrogen, a saturated and unsaturated hydrocarbyl group containing 1 to 8 carbon atoms or C₆ to C₁₀ aryl. In another aspect, R⁸⁵ is selected from a hydrogen, a C₁ to C₈ alkyl group, a C₂ to C₈ alkenyl group, or phenyl. Exemplary acids are, but are not limited to, formic acid, acetic acid, propionic acid, sorbic acid, caprylic acid, and benzoic acid, and mixtures thereof.

In another aspect, suitable acids include but are not limited to, oxalic acid, succinic acid, glutaric acid, adipic acid, azelaic acid, maleic acid, fumaric acid, lactic acid, glyceric acid, tartronic acid malic acid, tartaric acid, gluconic acid, citric acid, ascorbic acid, salicylic acid, phthalic acid, mandelic acid, benzilic acid, and mixtures thereof.

Salts of the foregoing acids are also useful as long as they retain efficacy at low pH values. Suitable salts include the alkali metal (e.g., sodium, potassium, calcium) and ammonium salts of the acids enumerated above.

The acid based preservatives and/or their salts can be used alone or in combination with non-acidic preservatives typically employed in personal care, home care, health care, and institutional and industrial care products.

The preservatives may comprise from 0.01 wt. % to 3.0 wt. % in one aspect, or from about 0.1 wt. % to about 1 wt. %, or from about 0.3 wt. % to about 1 wt. %, of the total weight of the hair care composition.

### Perfumes and Fragrances

Fragrance and perfume components that may be used in the exemplary composition to mask the odor of any of the various components in the hair conditioning composition or to give the composition an aesthetically pleasing fragrance. In one aspect, suitable fragrances and perfumes include natural and synthetic fragrances, perfumes, scents, and essences and any other substances which emit a fragrance. As the natural fragrances, there are those of vegetable origin, such as oil extracts from flowers (e.g., lily, lavender, rose, jasmine, neroli, ylang-ylang), stems and leaves (geranium, patchouli, petitgrain, peppermint), fruits (aniseed, coriander, fennel, mace, needle juniper), fruit skin (bergamot, lemon, orange), roots (angelica, celery, cardamom, costus, iris, sweet flag), woods (pine tree, sandalwood, guaiacum wood, cedar, rosewood, cinnamon), herbs and grasses (tarragon, lemongrass, sage, thyme), needles and twigs (spruce, pine, European red pine, stone pine), and resins and balsam (galbanum, elemi, benzoin, myrrh, frankincense, opopanax), and those of animal origin, such as musk, civet, castoreum, ambergris, or the like, and mixtures thereof.

Examples of synthetic fragrances and perfumes are the aromatic esters, ethers, aldehydes, ketones, alcohols, and hydrocarbons including benzyl acetate, phenoxyethyl isobutylate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styralyl propionate, and benzyl salicylate; benzylethyl ether; straight chain alkanals having 8 to 18 carbon atoms, citral, citronellal, citronellyloxyaldehyde, cyclamen aldehyde, hydroxycitronellal, lilial, and bougeonal; ionone compounds, α-isomethyl ionone, and methyl cedryl ketone; anethole, citronellol, eugenol, isoeugenol, geraniol, lavandulol, nerolidol, linalool, phenylethyl alcohol, and terpineol, alpha-pinene, terpenes (e.g., limonene), and balsams, and mixtures thereof.

The amount of fragrance agent or perfume employed can be any amount suitable to mask a particular odor or to impart a desired aesthetically pleasing aroma, fragrance or scent. In one aspect, the amount of fragrance agent can range from about 0.05 wt. % to about 10 wt. %, from about 0.1 wt. % to about 5 wt. % in another aspect, from about 0.5 wt. % to about 3.5 wt. % in still another aspect, and from about 1 wt. % to about 2.5 wt. % in a further aspect, based on the total weight of the composition.

In one embodiment, a composition of the disclosed technology is a clear composition that is stable to phase or ingredient separation at a temperature of about 25° C. for an indefinite period of time. For example, a clear conditioning composition of the present technology has demonstrated sufficient stability to phase and ingredient separation at temperatures normally found in commercial product storage and shipping to remain unaffected for periods of one year or more.

The conditioning compositions of the disclosed technology can be in the form of rinse-off products or leave-on products, and can be formulated in a wide variety of product forms, including but not limited to creams, gels, emulsions, mousses and sprays. In one aspect, the conditioning composition of the disclosed technology is suitable for rinse-off hair conditioner.

In one aspect, the conditioning composition of the disclosed technology is applied to the hair comprising the following steps: (i) after shampooing hair, applying to the hair an effective amount of the conditioning composition for conditioning the hair; and (ii) then rinsing the hair. Conditioning can be done right after shampooing when the hair is still wet or conditioning can be done separately from shampooing. The composition is topically applied to the wetted hair and then rinsed off. Application to the hair typically includes working the conditioning composition through the hair with the fingers to ensure complete coverage. An effective amount of the conditioning agent to apply ranges from about 0.1 ml to about 2 ml per 10 g of hair in one aspect, and from about 0.2 ml to about 1.5 ml per 10 g of hair in another aspect.

The hair conditioning compositions of the disclosed technology may be prepared by any known technique. The formulation of hair conditioning compositions are well-known in the formulation art and include conventional formulation and mixing techniques. Given the pH independent nature of the nonionic, amphiphilic polymer disclosed herein, it can be added at any point during the commercial production process of the conditioning product.

The disclosed technology is illustrated by the following examples that are merely for the purpose of illustration and are not to be regarded as limiting the scope of the technology or the manner in which it can be practiced. Unless specifically indicated otherwise, parts and percentages are given by weight.

### Test Methodology

### Yield Stress

The yield stress values of these polymers are determined by oscillatory and steady shear measurements on a controlled stress rheometer (TA Instruments AR1000N rheometer, New Castle, Del.) utilizing parallel plate geometry (40 mm stainless steel plate with a 1000 μm gap) at 25° C. The oscillatory measurements are performed at a fixed frequency of 1 rad/sec. The elastic and viscous moduli (G′ and G″ respectively) are obtained as a function of increasing stress amplitude. The stress corresponding to the crossover of G′ and G″ is noted as the yield stress.

### Brookfield Viscosity

Brookfield rotating spindle method (all viscosity measurements reported herein are conducted by the Brookfield method whether mentioned or not): The viscosity measurements are calculated in mPa·s, employing a Brookfield rotating spindle viscometer, Model RVT (Brookfield Engineering Laboratories, Inc.), at about 20 revolutions per minute (rpm), at ambient room temperature of about 20 to 25° C. (hereafter referred to as viscosity). Spindle sizes are selected in accordance with the standard operating recommendations from the manufacturer. Generally, spindle sizes are selected as follows:

| Spindle Size No. | Viscosity Range (mPa · s) |
|---|---|
| 1 | 1-50 |
| 2 | 500-1,000 |
| 3 | 1,000-5,000 |
| 4 | 5,000-10,000 |
| 5 | 10,000-20,000 |
| 6 | 20,000-50,000 |
| 7 | >50,000 |

The spindle size recommendations are for illustrative purposes only. The artisan of ordinary skill in the art will select a spindle size appropriate for the system to be measured.

### Clarity

The clarity (turbidity) of a composition is determined in Nephelometric Turbidity Units (NTU) employing a nephelometric turbidity meter (Mircro 100 Turbidimeter, HF Scientific, Inc.) at ambient room temperature of about 20 to 25° C. Distilled water (NTU=0) is utilized as a standard. Six dram screw cap vials (70 mm×25 mm) are filled almost to the top with test sample and centrifuged at 100 rpm until all bubbles are removed. Upon centrifugation, each sample vial is wiped with tissue paper to remove any smudges before placement in the turbidity meter. The sample is placed in the turbidity meter and a reading is taken. Once the reading stabilizes the NTU value is recorded. The vial is given one-quarter turn and another reading is taken and recorded. This is repeated until four readings are taken. The lowest of the four readings is reported as the turbidity value.

### Sensory Panel Testing of Conditioning Attributes

Conditioner formulations are compared by a trained panel (12 panelists) for conditioning attributes using a forced choice test design between two treated hair tresses. Hair tresses treated with a hair conditioning formulation according to the present technology (Formulation 1) are compared to hair tresses treated with a commercially available conditioning product purchased at retail (Formulation 2). Each panelist is asked to indicate which tress performs better for each of 5 sensory attributes evaluated in comparing the two formulations on the treated hair tresses. The sensory attributes evaluated by the panel include wet sensory attributes (evaluated immediately after the tresses are conditioned and rinsed), i.e., (1) ease of wet combing (after 3 comb strokes), (2) wet feel (relative slippery feel of the tress), and dry sensory attributes (evaluated after the tresses have been conditioned and rinsed followed by air drying under 50% relative humidity for 12 hrs.), i.e., (3) ease of dry combing (3 comb strokes), (4) dry feel (relative soft feel of the tress) and (5) static fly away (relative static repulsion of hair fibers after 3 comb strokes).

The test protocol utilizes a matrix design of 6 treated tresses (3 replicates for each of test Formulations 1 (disclosed technology) and Formulation 2 (commercial product). The test matrix allows for the direct blind comparison of the 3 replicate treated tresses of Formulation 1 (tress numbers 1, 2, and 3) versus the 3 replicate treated tresses of Formulation 2 (tress numbers 4, 5, and 6). By permutation of the 3 replicate treatments for each of Formulations 1 and 2, nine comparisons of paired tresses (Formulation 1 versus Formulation 2) are possible. Each panelist evaluates 6 paired combinations defined by the matrix illustrated in the table below (each panelist evaluates the paired combinations in one row of the matrix).

Comparison

| Panelist | 1 | 2 | 3 | 4 | 5 | 6 |
|---|---|---|---|---|---|---|
| 1 | 1 v 5 | 4 v 2 | 5 v 3 | 2 v 6 | 3 v 4 | 6 v 1 |
| 2 | 3 v 6 | 5 v 1 | 6 v 2 | 1 v 4 | 2 v 5 | 4 v 3 |
| 3 | 2 v 4 | 6 v 3 | 4 v 1 | 3 v 5 | 1 v 6 | 5 v 2 |
| 4 | 4 v 2 | 5 v 1 | 3 v 4 | 1 v 6 | 2 v 5 | 6 v 3 |
| 5 | 5 v 3 | 6 v 2 | 1 v 5 | 2 v 4 | 3 v 6 | 4 v 1 |
| 6 | 6 v 1 | 4 v 3 | 2 v 6 | 3 v 5 | 1 v 4 | 5 v 2 |
| 7 | 5 v 2 | 3 v 6 | 2 v 4 | 6 v 1 | 4 v 3 | 1 v 5 |
| 8 | 6 v 3 | 1 v 4 | 3 v 5 | 4 v 2 | 5 v 1 | 2 v 6 |
| 9 | 4 v 1 | 2 v 5 | 1 v 6 | 5 v 3 | 6 v 2 | 3 v 4 |
| 10 | 1 v 4 | 3 v 6 | 4 v 2 | 5 v 3 | 6 v 1 | 2 v 5 |
| 11 | 3 v 5 | 2 v 4 | 5 v 1 | 6 v 2 | 4 v 3 | 1 v 6 |
| 12 | 2 v 6 | 1 v 5 | 6 v 3 | 4 v 1 | 5 v 2 | 3 v 4 |

The preference of the panelist for each attribute in a paired tress combination is recorded by an independent data collector, and the sum of the number of times that tresses 1, 2, and 3 (Formulation 1) and tresses 4, 5, and 6 (Formulation 2) are selected as superior for a sensory attribute is tabulated and used for a statistical analysis for that particular attribute. The statistical analysis, (a Z-value calculation of preference of Formulation 1 versus Formulation 2) is used to determine the level of confidence that Formulation 1 is statistically different (better or worse) for the selected sensory attribute versus Formulation 2. The wet attributes are evaluated first where wet feel is evaluated followed by wet combing. Once the tresses are dried, the panelists come back to evaluate dry feel followed by dry combing and static fly away.

### Hair Tress Preparation Procedure for Sensory Panel Testing

Hair tresses (European virgin brown hair) weighing 2.5 g (dry wt.) are prewashed with a stripping shampoo (surfactant iso-propanol mixture containing 10 wt. % sodium lauryl sulfate and 10 wt. % iso-propanol) and thoroughly rinsed under warm tap water to remove the shampoo. Excess water is removed by pinching each tress between the index finger and the middle finger and gently pulling the tress through the gap of the fingers. The damp tress is placed on top of the weighing dish and 0.5 g of the test hair conditioning formulation is applied evenly down the length of the hair tress. The conditioner is massaged into the tress from the root to the tip of the hair tress for 60 seconds and the treated tress is allowed to rest for an additional 60 seconds. The tress is then rinsed under warm tap water (37° C., +2° C.; flow rate of 1.3 gal/min) for approximately 30 seconds. While rinsing, the tress is combed through its length at least 20 to 25 times to ensure that all residual conditioner is removed. The treatment step is repeated a second time for a total of two washes/rinses.

### Example 1

An emulsion polymer was polymerized as follows. A monomer premix was made by mixing 140 grams of deionized (D.I.) water, 3.75 grams of 40% (active) sodium alpha olefin sulfonate (AOS) aqueous solution, 175 grams (EA), 70.5 grams of (n-BA), 225 grams of (HEMA) and 33.3 grams of (BEM). Initiator A was separately prepared by mixing 3.57 grams of 70% t-butyl hydrogen peroxide (TBHP) in 40 grams of D.I. water. Reductant A was separately prepared by dissolving 0.13 grams of erythorbic acid in 5 grams of D.I. water, and Reductant B was separately prepared by dissolving 2.5 grams of erythorbic acid in 100 grams of D.I. water. A 3-liter reactor was charged with 800 grams of D.I. water, 10 grams of 40% AOS and 25 grams of Selvol™ 502 polyvinylalcohol and then heated to 70° C. under a nitrogen blanket with mild agitation. After holding the reactor at 70° C. for one hour the reactor was cooled to 65° C. Initiator A was then added to the reactor followed by adding Reductant A to the reactor. After about 1 minute, the monomer premix was metered into the reaction vessel over a period of 180 minutes. About 3 minutes after the start of monomer premix metering, Reductant B was metered into the reactor over a period of 210 minutes. The reaction temperature was maintained at 65° C. At about 110 minutes after the monomer premix metering, the premix metering was stopped for 10 minutes, and then 0.45 grams of (APE) in 4.05 grams of (n-BA) was added to the monomer premix. After 10 minutes the premix metering was re-started. After completion of Reductant B feed, the temperature of the reaction vessel was maintained at 65° C. for 60 minutes. The reactor was then cooled to 60° C. A solution of 1.96 grams of 70% TBHP and 0.13 grams of 40% AOS in 15 grams of D.I. water was added to the reactor. After 5 minutes, a solution of 1.27 grams of erythorbic acid in 15 grams of D.I. water was added to the reactor. The reactor temperature was maintained at 60° C. After 30 minutes, a solution of 1.96 grams of 70% TBHP and 0.13 grams of 40% AOS in 15 grams of D.I. water was added to the reactor. After 5 minutes, a solution of 1.27 grams of erythorbic acid in 15 grams of D.I. water was added to the reactor. The reactor temperature was maintained at 60° C. for about 30 minutes. Then the reactor was cooled to room temperature (22° C.) and filtered through 100-micron cloth. The pH of the resulting emulsion was adjusted to 4.5 with 10% ammonium hydroxide in water. The polymer emulsion had a solids content of 29.8%, a Brookfield viscosity of 18 cps, and a particle size of 110 nm as measured on a Nicomp 380 nanoparticle size analyzer (dynamic light scattering method), Particle Sizing Systems, Port Richey, Fla.

### Example 2

An emulsion polymer was polymerized as follows. A monomer premix was made by mixing 140 grams of D.I. water, 5 grams of RS-1618, 175 grams of EA, 75 grams of n-butyl acrylate n-BA, 225 grams of HEMA and 33.3 grams of BEM. Initiator A was separately prepared by dissolving 5 grams of 2,2′-azobis[2-methyl-N-(2-hydroxyethyl)propionamide] (Azo VA-086 from Wako Pure Chemical Industries, Ltd.) in 40 grams of water. Initiator B was separately prepared by dissolving 2.5 grams of Azo VA-086 in 100 grams of D.I. water. A 3-liter reactor was charged with 800 grams of D.I. water, 5 grams of 40% AOS and 10 grams of Selvol 203 from Sekisui and the charge was then heated to 87° C. under a nitrogen blanket with proper agitation. After holding the reactor at 87° C. for one hour, Initiator A was added to the reactor. After about 1 minute the monomer premix was metered to the reaction vessel over a period of 120 minutes. About 3 minutes after the start of monomer premix metering, Initiator B was metered to the reactor over a period of 150 minutes. The reaction temperature was kept at 87° C. After completion of Initiator B feed, the temperature of the reaction vessel was maintained at 87° C. for 60 minutes. The reactor was then cooled to 49° C. A solution of 0.61 grams of 70% TBHP and 0.29 grams of 40% AOS in 15 grams of water was added to the reactor. After 5 minutes, a solution of 0.59 grams of erythorbic acid in 15 grams of water was added to the reactor. The reactor was maintained at 49° C. After 30 minutes, a solution of 0.69 grams of 70% TBHP and 0.29 grams of 40% AOS in 15 grams of water was added to the reactor. After 5 minutes, a solution of 0.59 grams of erythorbic acid in 15 grams of water was added to the reactor. The reactor was maintained at 49° C. for about 30 minutes. The reactor then was cooled to room temperature (22° C.) and filtered through 100-micron cloth. The pH of the resulting emulsion was adjusted to 4.5 with 10% ammonium hydroxide in water. The polymer emulsion had a solids content of 30.2%, a Brookfield viscosity of 27 cps, and a particle size of 79 nm as measured on a Nicomp 380 nanoparticle size analyzer (dynamic light scattering method), Particle Sizing Systems, Port Richey, Fla.

### Example 3

An exemplary conditioning composition according to the disclosed technology was formulated and prepared from the components and in accordance with the procedures set forth below.

| Component | Ingredients | Function | Wt. % |
|---|---|---|---|
| A | D.I Water | Diluent | 33.62 |
| | Polymer of Ex. 2 | Rheology | 12.0 |
| | (25% Total Solids) | Modifier | |
| B | Deionized (D.I.) Water | Diluent | 41.11 |
| | Cetrimonium Chloride¹ | Conditioner | 9.6 |
| | (25% Active) | | |
| | PEG-120 Methyl Glucose Trioleate | Thickener | 3.0 |
| | (and) Propylene Glycol (and) | | |
| | Water² (40% Active) | | |
| | Stearyl Ethylhexyldimonium | Auxiliary | 0.62 |
| | Methosulfate³ | Conditioner | |
| | (83% Active) | | |
| | Methylchloroisothiazolinone (and) | Preservative | 0.05 |
| | Methylisothiazolinone⁴ | | |

¹Genamin ® CTAC 25 quaternium compound, Clariant Corporation
²Glucamate ™ LT ethoxylated methyl glucoside ester, Lubrizol Advanced Materials, Inc.
³Arquad ® PC HTL-8 MS quaternium compound, Akzo Nobel N.V.
⁴Kathon ™ CG preservative, The Dow Chemical Company

Procedure: The Part A components were mixed by adding D.I. water to the polymer in a suitable mixing vessel equipped with a mechanical stirrer. The components were mixed at 150 rpm until uniform. The Part B components were added to a separate mixing vessel and mixed until homogenous. The Part B mixture was then added to the Part A mixture and mixed at 450 rpm until a uniform composition was obtained.

The composition had a Brookfield viscosity (B.V.) of 9600 mPa·s measured after 24 hours at ambient room temperature (approximately 23° C.) at 20 rpm, and a yield stress (Y.S.) value of 7.1 Pa.

### Example 4

Conditioning compositions were formulated with the polymer of Example 1, a quaternium compound, a fatty acid ester of an ethoxylated alkyl glucoside, and water (q.s. to 100 wt. %) were prepared in a similar fashion to the procedure set forth in Example 3. For each formulation the amount of polymer and quaternium compound were kept constant, while the amount of ethoxylated glucoside ester was incrementally increased in each formulation as set forth in the table below. The pH for all compositions ranged from 2.8 to 3.5. Brookfield viscosity (measured 24 hrs. after formulation at ambient room temperature), yield value, and clarity properties were measured as reported below.

| Polymer | | Ethoxylated | | | |
|---|---|---|---|---|---|
| Ex. 1 | CTAC² | Glucoside Ester³ | Y.S. | B.V.⁴ | Clarity |
| (wt. %)¹ | (wt. %)¹ | (wt. %)¹ | (Pa) | (mPa · s) | (NTU) |
| 3.0 | 5.0 | 0.2 | 1.3 | 1160 | 27.4 |
| 3.0 | 5.0 | 0.5 | 2.0 | 2380 | 29.9 |
| 3.0 | 5.0 | 0.7 | 2.3 | 3080 | 28.4 |
| 3.0 | 5.0 | 1.0 | 2.4 | 3600 | 29.6 |
| 3.0 | 5.0 | 1.5 | 3.0 | 6040 | 31.9 |
| 3.0 | 5.0 | 2.0 | 3.4 | 8100 | 32.9 |
| 3.0 | 5.0 | 2.5 | 3.7 | 14180 | 31.5 |

¹Wt. % based on active material
²Genamin ® CTAC 25 Cetrimonium Chloride, Clariant Corporation
³Glucamate ™ LT LT ethoxylated methyl glucoside ester, Lubrizol Advanced Materials, Inc.
⁴Measured 24 hours after formulating at ambient room temperature (approximately 23° C.) at 20 rpm

As the concentration of ethoxylated glucoside ester increases viscosity increases accordingly, while clarity values remains fairly constant.

### Example 5 (Comparative)

Compositions containing the polymer of Example 1, Cetrimonium Chloride, and water were formulated utilizing the amounts set forth in the table below. Brookfield viscosity (measured 24 hrs. after formulation at ambient room temperature), yield value, and clarity properties were measured as reported below.

| Polymer Ex. 1 | CTAC² | | Y.S. | B.V.³ | Clarity |
|---|---|---|---|---|---|
| (wt. %)¹ | (wt. %)¹ | pH | (Pa) | (mPa · s) | (NTU) |
| 1.5 | 3 | 3.8 | 0.00 | 40 | 88.4 |
| 2.0 | 3 | 3.6 | 0.00 | 90 | 42.3 |
| 2.5 | 3 | 3.5 | 0.22 | 130 | 32.1 |
| 3.0 | 3 | 3.5 | 0.39 | 160 | 26.7 |
| 4.0 | 3 | 3.7 | 0.31 | 180 | 68.0 |

¹Wt. % based on active material
²Genamin ® CTAC 25 Cetrimonium Chloride, Clariant Corporation
⁴Measured 24 hours after formulating at ambient room temperature (approximately 23° C.) at 20 rpm

In the absence of the ethoxylated glucoside ester component, viscosity does not build and clarity becomes unstable.

### Example 6

A conditioning composition was formulated from the components set forth in the table below.

| Component | Ingredients | Function | Wt. % |
|---|---|---|---|
| A | D.I Water | Diluent | 33.8 |
| | Polymer of Ex. 2 | Rheology | 12.0 |
| | (25% Total Solids) | Modifier | |
| B | Deionized (D.I.) Water | Diluent | 41.11 |
| | Cetrimonium Chloride¹ | Conditioner | 9.6 |
| | (25% Active) | | |
| | PEG-120 Methyl Glucose Trioleate | Thickener | 3.0 |
| | (and) Propylene Glycol (and) | | |
| | Water² (40% Active) | | |
| | Stearyl Ethylhexyldimonium | Auxiliary | 0.62 |
| | Methosulfate³ | Conditioner | |
| | (83% Active) | | |
| | Methylchloroisothiazolinone (and) | Preservative | 0.05 |
| | Methylisothiazolinone⁶ | | |
| | Aqueous Sodium Hydroxide | pH Adjustor | 5.0 |
| | (20 wt. %) | | |

¹Genamin ® CTAC 25 quaternium compound, Clariant Corporation
²Glucamate ™ LT ethoxylated methyl glucoside ester, Lubrizol Advanced Materials, Inc.
³Arquad ® PC HTL-8 MS quaternium compound, Akzo Nobel N.V.
⁴Kathon ™ CG preservative, The Dow Chemical Company

Procedure: The Part A components were mixed by adding D.I. water to the polymer in a suitable mixing vessel equipped with a mechanical stirrer. The components were mixed at 150 rpm until uniform. The Part B components were added to a separate mixing vessel and mixed until homogenous. The Part B mixture was then added to the Part A mixture and mixed at 450 rpm until a uniform composition was obtained. The composition had a translucent appearance, a 24 hr. Brookfield viscosity of 8,100 mPa·s, and a yield stress of 7.1 Pa.

Hair tresses treated with the obtained experimental conditioning composition were compared to hair tresses treated with a leading commercially available conditioning product purchased at retail for sensory attributes in accordance with the hair tress preparation and sensory panel testing protocols set forth above. The commercial conditioner listed the following ingredients on the product label: Aqua, Stearyl Alcohol, Cetyl Alcohol, Behentrimonium Chloride, Cetearyl Ethylhexyldimonium Methosulfate, Bis-Aminopropyl Dimethicone, Parfum, Benzyl Alcohol, Isopropyl Alcohol, Disodium EDTA, Panthenol, Panthenyl Ethyl Ether, Butylphenyl Methylpropional, Linalool, Hexyl Cinnamal, Limonene, Magnesium Nitrate, Methylchloroisothiazolinone, Magnesium Chloride, and Methylisothiazol inone.

The results of the panel test indicated that hair tresses treated with the disclosed technology formulation has better dry combing attributes (>95% confidence level), better static fly away attributes (>95% confidence level) than the comparative formulation, with no statistical differences in wet combing, wet feel and dry feel attributes.

### Example 7

A conditioning composition was formulated from the components set forth in the table below.

| Component | Ingredients | Function | Wt. % |
|---|---|---|---|
| A | D.I Water | Diluent | 33.8 |
| | Polymer of Ex. 2 | Rheology | 12.0 |
| | (25% Total Solids) | Modifier | |
| B | Deionized (D.I.) Water | Diluent | 41.11 |
| | Cetrimonium Chloride¹ | Conditioner | 9.6 |
| | (25% Active) | | |
| | PEG-120 Methyl Glucose Trioleate | Thickener | 3.0 |
| | (and) Propylene Glycol (and) | | |
| | Water² (40% Active) | | |
| | Stearyl Ethylhexyldimonium | Auxiliary | 0.62 |
| | Methosulfate³ | Conditioner | |
| | (83% Active) | | |
| | Lauryl Methyl Gluceth-10 | Auxiliary | 0.5 |
| | Hydroxypropyldimonium | Cationic | |
| | Chloride⁴ | Surfactant | |
| | Silicone Quaternium-8⁵ | Auxiliary | 1.0 |
| | | Conditioning | |
| | | Agent | |
| | Methylchloroisothiazolinone (and) | Preservative | 0.05 |
| | Methylisothiazolinone⁶ | | |

¹Genamin ® CTAC 25 quaternium compound, Clariant Corporation
²Glucamate ™ ethoxylated methyl glucoside ester, Lubrizol Advanced Materials, Inc.
³Arquad ® PC HTL-8 MS quaternium compound, Akzo Nobel N.V.
⁴Glucquat ™ 125 cationic surfactant, Lubrizol Advanced Materials, Inc.
⁵SilSense ™ Q-Plus conditioning agent
⁶Kathon ™ CG preservative, The Dow Chemical Company

Procedure: The Part A components were mixed by adding D.I. water to the polymer in a suitable mixing vessel equipped with a mechanical stirrer. The components were mixed at 150 rpm until uniform. The Part B components were added to a separate mixing vessel and mixed until homogenous. The Part B mixture was then added to the Part A mixture and mixed at 450 rpm until a uniform composition was obtained. An aqueous solution of sodium hydroxide (20 wt. %) was added to the composition to adjust the pH to 4.5.

The composition had a Brookfield viscosity (B.V.) of 12,300 mPa·measured after 24 hours at ambient room temperature (approximately 23° C.) at 20 rpm.

Hair tresses treated with the obtained experimental conditioning composition were compared to hair tresses treated with a leading commercially available conditioning product purchased at retail for sensory attributes in accordance with the hair tress preparation and sensory panel testing protocols set forth above. The commercial conditioner listed the following ingredients on the product label: Water, Stearyl Alcohol, Cetyl Alcohol, Stearamidopropyl Dimethylamine, Fragrance, Benzyl Alcohol, Dicetyldimonium Chloride, Glutamic Acid, Bis-Aminopropyl Dimethicone, Disodium EDTA, Panthenol, Panthenyl Ethyl Ether, Citric Acid, Methylchloroisothiazolinone, and Methylisothiazolinone

The results of the panel test indicated that hair tresses treated with the disclosed technology formulation has better wet combing attributes (>95% confidence level), better dry combing attributes (>99% confidence level) and better static fly away attributes (>95% confidence level) than the comparative formulation, with no statistical differences in wet feel and dry feel attributes.

### Example 8

Comparative base formulations were prepared from the components and amounts listed in the table below. Each of the formulations were measured pH, Brookfield viscosity and clarity.

| Polymer | | Ethoxylated | | | |
|---|---|---|---|---|---|
| Ex. 2 | CTAC² | Glucoside Ester³ | | B.V.⁴ | Clarity |
| (wt. %)¹ | (wt. %)¹ | (wt. %)¹ | pH | (mPa · s) | (NTU) |
| — | 2.40% | — | 3.13 | Water-Like | 0.13 |
| — | 2.40% | 1.20% | 3.16 | Water-Like | 0.41 |
| 3% | 2.40% | — | 2.92 | 1100 | 13.2 |
| 3% | 2.40% | 1.20% | 2.99 | 6300 | 19.8 |

¹Wt. % based on active material
²Genamin ® CTAC 25 Cetrimonium Chloride, Clariant Corporation
³Glucamate ™ LT ethoxylated methyl glucoside ester, Lubrizol Advanced Materials, Inc.
⁴Measured 24 hours after formulating at ambient room temperature (approximately 23° C.) at 20 rpm

From the viscosity measurements it is evident that both the nonionic, amphiphilic, emulsion polymer component and the ethoxylated glucoside ester components are necessary to build viscosity.

### Example 9

Compositions containing the polymer of Example 1, Cetrimonium Chloride, ethoxylated glucoside ester and water were formulated utilizing the amounts set forth in the table below. The amounts of polymer and CTAC were kept constant, while the amount of ethoxylated glucoside ester was increasingly varied. Brookfield viscosity, yield value, and clarity properties were measured as reported below.

| Polymer | | Ethoxylated | | | |
|---|---|---|---|---|---|
| Ex. 2 | CTAC² | Glucoside Ester³ | Y.S. | B.V.⁴ | Clarity |
| (wt. %)¹ | (wt. %)¹ | (wt. %)¹ | (Pa) | (mPa · s) | (NTU) |
| 3 | 5 | 0.2 | 1.3 | 1160 | 27.4 |
| 3 | 5 | 0.5 | 2.0 | 2380 | 29.9 |
| 3 | 5 | 0.7 | 2.3 | 3080 | 28.4 |
| 3 | 5 | 1.0 | 2.4 | 3600 | 29.6 |
| 3 | 5 | 1.5 | 3.0 | 6040 | 31.9 |
| 3 | 5 | 2.0 | 3.4 | 8100 | 32.9 |
| 3 | 5 | 2.5 | 3.7 | 14,180 | 31.5 |

¹Wt. % based on active material
²Genamin ® CTAC 25 Cetrimonium Chloride, Clariant Corporation
³Glucamate ™ LT ethoxylated methyl glucoside ester, Lubrizol Advanced Materials, Inc.
⁴Measured 24 hours after formulating at ambient room temperature (approximately 23° C.) at 20 rpm

The results indicate that yield stress and Brookfield viscosity values increase as the amount of ethoxylated glucoside ester increases, while the clarity values remain relatively stable.

1. A hair conditioning composition comprising:
(a) at least one cationic compound selected from a mono(C₁₂-C₂₂) akyl tri(C₁-C₃) alkyl quaternary ammonium salt in an amount ranging from about 1.0 to about 10 wt. %;
(b) at least one fatty acid ester of an ethoxylated alkyl glucoside in an amount ranging from about 0.5 to about 10 wt. %;
(c) a nonionic, amphiphilic, emulsion polymer in an amount ranging from about 0.5 to about 6.5 wt. %; and
(d) an aqueous carrier;
wherein said emulsion polymer is prepared from a polymerizable monomer mixture comprising at least one hydrophilic monomer and at least one hydrophobic monomer, wherein said hydrophilic monomer is selected from hydroxy(C₁-C₅)alkyl (meth)acrylates, wherein said hydrophobic monomer is selected from esters of (meth)acrylic acid with alcohols containing 1 to 30 carbon atoms, vinyl esters of aliphatic carboxylic acids containing 1 to 22 carbon atoms, vinyl ethers of alcohols containing 1 to 22 carbon atoms, vinyl aromatic monomers, vinyl halides, vinylidene halides, associative monomers, semi-hydrophobic monomers, or mixtures thereof.

2. A hair conditioning composition of claim 1 further comprising (e) a silicone conditioning agent.

3. A hair conditioning composition of claim 1 further comprising a conditioning agent selected from a cationic compound different from (a), a cationic polymer, a hydrocarbon oil, a natural oil, an ester oil, a synthetic wax, and combinations thereof.

4. A hair conditioning composition of claim 1 wherein component (a) is represented by the formula:
(R′″)(R″)(R′)(R)N⁺A⁻
where R represents a C₁₂-C₂₂ alkyl radical, R′, R″, R′″ independently represent a C₁-C₃ alkyl, and A⁻ is an anion selected from chloride, bromide and methosulfate.

5. A hair conditioning composition of claim 1 wherein component (a) is represented by the formula: 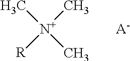
where R represents a C₁₂-C₂₂ alkyl radical, and A⁻ is an anion selected from chloride, bromide and methosulfate.

6. A hair conditioning composition of claim 1 wherein component (a) is selected from cetrimonium chloride, cetrimonium bromide, cetrimonium methosulfate, steartrimonium chloride, behentrimonium chloride, behentrimonium methosulfate, and mixtures thereof.

7. A hair conditioning composition of claim 1 wherein component (b) is represented by the following structure: 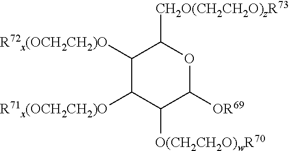
wherein R⁶⁹ represents C₁ to C₅ alkyl, R⁷⁰, R⁷¹, R⁷² and R⁷³ independently represent hydrogen or an acyl substituent represented by —C(O)R⁷⁴, where R⁷⁴ is selected from C₅ to C₂₁ alkyl, C₅ to C₂₁ alkenyl, and combinations thereof, subject to the proviso that at least one of R⁷⁰ to R⁷⁴ must be selected from said acyl substituent, and wherein the sum of w+x+y+z ranges from about 50 to about 400.

8. A hair conditioning composition of claim 7 wherein component (b) is substituted with from about 1 to about 4 moles of said acyl substituent.

9. A hair conditioning composition of claim 8 wherein said acyl substituent is an acyl residue of a fatty acid selected from caproic acid, enanthic acid, caprylic acid, pelargonic acid, capric acid, undecanoic acid, lauric acid, myristic acid, palmitic acid, palmitoleic acid, stearic acid, isostearic acid, oleic acid, ricinoleic acid, vaccenic acid, linoleic acid, α-linolenic acid, γ-linolenic acid, arachidic acid, gadoleic acid, arachidic acid, 5,8,11,14,17-eicosapentaenoic acid, behenic acid, erucic acid, 4,7,10,13,16,19-docosahexaenoic acid, and combinations thereof.

10. A hair conditioning composition of claim 7 wherein R⁶⁹ is selected from methyl, ethyl, propyl, butyl and amyl.

11. A hair conditioning composition of claim 7 wherein R⁶⁹ is methyl and at least one of R⁷⁰ to R⁷³ is acyl residue of oleic acid.

12. A hair conditioning composition of claim 2, wherein said silicone conditioning agent is non-volatile silicone selected from a silicone oil, silicone gum, silicone resin and mixtures thereof.

13. A hair conditioning composition of claim 12 further comprising a volatile silicone.

14. A hair conditioning composition of claim 12, wherein said silicone oil is selected from a compound represented by the formula: 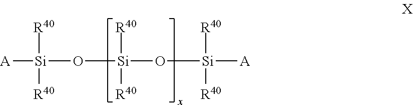
wherein A independently represents hydroxy, methyl, methoxy, ethoxy, propoxy, and phenoxy; R⁴⁰ independently represents methyl, ethyl, propyl, phenyl, methylphenyl, phenylmethyl; and x is an integer ranging from about 7 to about 8000.

15. A hair conditioning composition of claim 12 wherein said silicone conditioning agent is selected from polydimethylsiloxanes (dimethicones), polydiethylsiloxanes, polydimethyl siloxanes having terminal hydroxyl groups (dimethiconols), polymethylphenylsiloxanes, phenylmethylsiloxanes, amino functional polydimethylsiloxane (amodimethicone), non-ionic dimethicone copolyols, dimethicone copolyol esters, dimethicone copolyol quaternium nitrogen containing compounds, dimethicone copolyol phosphate esters, and mixtures thereof.

16. A hair conditioning composition of claim 15 wherein said amino functional silicone is selected from a compound represented by the formula: 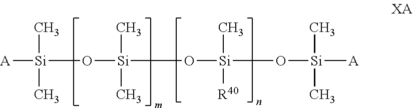
wherein A independently represents hydroxy, methyl, methoxy, ethoxy, propoxy, and phenoxy;
and R⁴⁰ is selected from:
—R⁴¹—N(R⁴²)CH₂CH₂N(R⁴²)₂;
—R⁴¹—N(R⁴²)₂;
—R⁴¹—N⁺(R⁴²)₃CA⁻; and
—R⁴¹—N(R⁴²)CH₂CH₂N(R⁴²)H₂CA⁻
wherein R⁴¹ is a linear or branched, hydroxyl substituted or unsubstituted alkylene or alkylene ether moiety containing 2 to 10 carbon atoms; R⁴² is hydrogen, C₁-C₂₀ alkyl (e.g, methyl), phenyl or benzyl; CA⁻ is a halide ion selected from chlorine, bromine, iodine and fluorine; and the sum of m+n ranges from about 7 to about 1000, subject to the proviso that m or n is not 0.

17. A hair conditioning composition of claim 2 wherein said silicone conditioning agent is present in an amount ranging from about 0.01 to about 20 wt. %, based on the weight of the total composition.

18. A hair conditioning composition of claim 2 wherein said silicone conditioning agent has a particle size ranging from about 0.003 to about 500 μm.

19. A hair conditioning composition of claim 2 wherein said silicone conditioning agent is in the form of emulsion droplets.

20. A hair conditioning composition of claim 3 wherein said cationic polymer is selected from Polyquaternium-4, Polyquaternium-6, Polyquaternium-10, Polyquatemium-11, Polyquarternium-16, Polyquaternium-24, Polyquaternium-28, Polyquaternium-29, Polyquaternium-32, Polyquaternium-33, Polyquatemium-35, Polyquatemium-37, Polyquarternium-39, Polyquaternium-44, Polyquaternium-46, Polyquaternium-47, Polyquaternium-52, Polyquaternium-53, Polyquarternium-55, Polyquaternium-59, Polyquatemium-61, Polyquaternium-64, Polyquaternium-65, Polyquaternium-67, Polyquaternium-69, Polyquaternium-70, Polyquaternium-71, Polyquaternium-72, Polyquaternium-73, Polyquaternium-74, Polyquaternium-76, Polyquaternium-77, Polyquaternium-78, Polyquaternium-79, Polyquaternium-80, Polyquaternium-81, Polyquaternium-82, Polyquaternium-84, Polyquaternium-85, Polyquatemium-87, and PEG-2-cocomonium chloride, Starch Hydroxypropyltrimonium Chloride, Guar Hydroxypropyltrimonium Chloride, Hydroxypropyl Guar Hydroxypropyltrimonium Chloride, and Cassia Hydroxypropyltrimonium Chloride, and mixtures thereof.

21. A hair conditioning composition of claim 3 wherein said cationic compound different from (a) is selected from a dialkyl quaternary ammonium compound, an asymmetric dialkyl quaternary ammonium compound, and mixtures thereof.

22. A hair conditioning composition of claim 21 wherein said dialkyl quaternary ammonium compound is selected from dicocodimonium chloride; dicocodimonium bromide; dimyristyldimonium chloride; dimyristyldimonium bromide; dicetyldimonium chloride; dicetyldimonium bromide; dicetylmethylbenzylmonium chloride; distearyldimonium chloride; distearyldimonium bromide; dimetyldi(hydrogenated tallow)monium chloride; hydroxypropylbisstearylmonium chloride; distearylmethylbenzylmonium chloride; dibehenyl/diarachidyldimonium chloride; dibehenyl/diarachidyldimonium bromide; dibehenyldimonium chloride; dibehenyldimonium bromide; dibehenyldimonium methosulfate; dibehenylmethylbenzylmonium chloride; dihydrogenated tallow benzylmonium chloride; dihydrogenated tallowethyl hydroxyethylmonium methosulfate; dihydrogenated tallow hydroxyethylmonium methosulfate; di-C₁₂-C₁₅ alkyldimonium chloride; di-C₁₂-C₁₈ alkyldimonium chloride; di-C₁₄-C₁₈ alkyldimonium chloride; dicocoylethyl hydroxyethylmonium methosulfate; disoyoylethyl hydroxyethylmonium methosulfate; dipalmitoylethyldimonium chloride; dihydrogenated palmoylethyl hydroxyethylmonium methosulfate; dihydrogenated tallowamidoethyl hydroxyethylmonium chloride; dihydrogenated tallowamidoethyl hydroxyethylmonium methosulfate; dihydrogenated tallowoylethyl hydroxyethylmonium methosulfate; distearoylethyl hydroxyethylmonium methosulfate; and mixtures thereof.

23. A hair conditioning composition of claim 21 wherein said asymmetric dialkyl quaternary ammonium compound is selected from stearylethylhexyldimonium chloride, stearylethylhexyldimonium bromide; stearyl ethylhexyl dimonium methosulfate; cetearyl ethylhexyldimonium methosulfate; and mixtures thereof.

24. A composition according to claim 1 wherein said hydroxy(C₁-C₅)alkyl (meth)acrylate is selected from at least one compound represented by the formula: 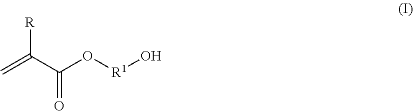
wherein R is hydrogen or methyl and R¹ is an divalent alkylene moiety containing 1 to 5 carbon atoms, wherein the alkylene moiety optionally can be substituted by one or more methyl groups.

25. (canceled)

26. (canceled)

27. A hair conditioning composition according to claim 1 wherein said ester of (meth)acrylic acid with alcohols containing 1 to 30 carbon is selected from at least one compound represented by the formula: 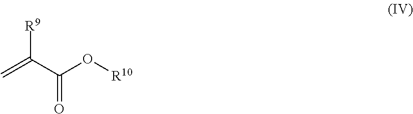
wherein R⁹ is hydrogen or methyl and R¹⁰ is C₁ to C₂₂ alkyl.

28. A hair conditioning composition according to claim 1 wherein said vinyl ester of aliphatic carboxylic acids containing 1 to 22 carbon atoms is selected from at least one compound represented by the formula: 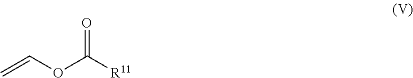
wherein R¹¹ is a C₁ to C₂₂ aliphatic group which can be an alkyl or alkenyl.

29. A hair conditioning composition according to claim 1 wherein said vinyl ether of alcohols containing 1 to 22 carbon atoms is selected from at least one compound represented by the formula: 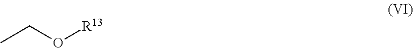
wherein R¹³ is a C₁ to C₂₂ alkyl.

30. A hair conditioning composition according to claim 1 wherein said associative monomer comprises (i) an ethylenically unsaturated end group portion; (ii) a polyoxyalkylene mid-section portion, and (iii) a hydrophobic end group portion containing 8 to 30 carbon atoms.

31. A hair conditioning composition according to claim 30 wherein said associative monomer is represented by formulas VII and/or VIIA: 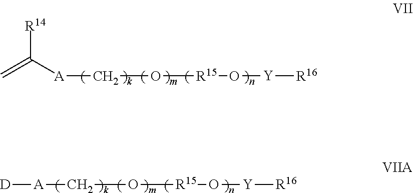
wherein R¹⁴ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; D represents a vinyl or an allyl moiety; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or C₄H₈, and combinations thereof; and n is an integer in the range of about 2 to about 150; Y is —R¹⁵O—, —R¹⁵NH—, —C(O)—, —C(O)NH—, —R¹⁵NHC(O)NH—, or —C(O)NHC(O)—; R¹⁶ is a substituted or unsubstituted alkyl selected from a C₈-C₃₀ linear alkyl, a C₈-C₃₀ branched alkyl, a C₈-C₃₀ carbocyclic alkyl, a C₂-C₃₀ alkyl-substituted phenyl, an aralkyl substituted phenyl, and an aryl-substituted C₂-C₃₀ alkyl; wherein the R¹⁶ alkyl group, aryl group, phenyl group optionally comprises one or more substituents selected from the group consisting of a hydroxyl group, an alkoxyl group, benzyl group styryl group, and a halogen group.

32. A hair conditioning composition according to claim 31 wherein said associative monomer is represented by formula VIIB: 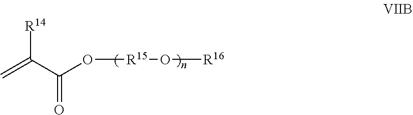
wherein R¹⁴ is hydrogen or methyl; R¹⁵ is a divalent alkylene moiety independently selected from C₂H₄, C₃H₆, and C₄H₈, and n represents an integer ranging from about 10 to about 60, (R¹⁵—O) can be arranged in a random or a block configuration; R¹⁶ is a substituted or unsubstituted alkyl selected from a C₈-C₃₀ linear alkyl, a C₈-C₃₀ branched alkyl, a C₈-C₃₀ carbocyclic alkyl, a C₂-C₃₀ alkyl-substituted phenyl, an aralkyl substituted phenyl, and an aryl-substituted C₂-C₃₀ alkyl, wherein the R¹⁶ alkyl group, aryl group, phenyl group optionally comprises one or more substituents selected from the group consisting of a hydroxyl group, an alkoxyl group, benzyl group styryl group, and a halogen group.

33. A hair conditioning composition according to claim 1 wherein said semi-hydrophobic monomer comprises (i) an ethylenically unsaturated end group portion; (ii) a polyoxyalkylene mid-section portion, and (iii) an end group portion selected from hydrogen or an alkyl group containing 1 to 4 carbon atoms.

34. A hair conditioning composition according to claim 33 wherein said semi-hydrophobic monomer is selected from at least one monomer represented by formulas VIII and IX: 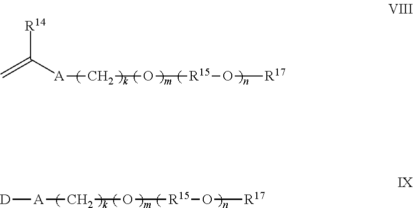
wherein R¹⁴ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or C₄H₈, and combinations thereof; and n is an integer in the range of about 2 to about 150; R¹⁷ is selected from hydrogen and a linear or branched C₁-C₄ alkyl group; and D represents a vinyl or an allyl moiety.

35. A hair conditioning composition according to claim 34 wherein said semi-hydrophobic monomer is selected from at least one monomer represented by formulas VIIIA and VIIIB:
CH₂═C(R¹⁴)C(O)O—(C₂H₄O)ₐ(C₃H₆O)_{b}—H VIIIA
CH₂═C(R¹⁴)C(O)O—(C₂H₄O)ₐ(C₃H₆O)_{b}—CH₃ VIIIB
wherein R¹⁴ is hydrogen or methyl, and “a” is an integer ranging from 0 or 2 to about 120, and “b” is an integer ranging from about 0 or 2 to about 120, subject to the proviso that “a” and “b” cannot be 0 at the same time.

36. A hair conditioning composition according to claim 35 wherein b is 0.

37. A hair conditioning composition according to claim 1 wherein said emulsion polymer is polymerized from a monomer mixture comprising at least 30 wt. % of said hydrophilic monomer(s) and at least 5 wt. % of said hydrophobic monomers.

38. A hair conditioning composition according to claim 1 wherein said monomer mixture comprises a crosslinking monomer which is present in an amount sufficient to be incorporated into said polymer from about 0.01 to about 1 wt. %, (based on the weight of the monomer composition).

39. A hair conditioning composition according to claim 38 wherein said crosslinking monomer contains an average of about 3 crosslinkable unsaturated moieties.

40. A hair conditioning composition according to claim 38 wherein said monomer mixture comprises a crosslinking monomer which is present in an amount sufficient to be incorporated into said polymer from about 0.01 to about 0.3 wt. %, (based on the weight of the monomer composition).

41. A hair conditioning composition according to claim 38 wherein the at least one crosslinking monomer is selected from polyallyl ethers of trimethylolpropane, polyallyl ethers of pentaerythritol, polyallyl ethers of sucrose, or mixtures thereof.

42. A hair conditioning composition according to claim 1 wherein said monomer mixture comprises a crosslinking reactive surfactant selected from at least one monomer represented by the formula:
R₁O—(CH₂CHR₂O)ₓ—(CH₂CH₂O)_{y}—(CH₂CHR₃O)_{z}—R₄ X
wherein R₁ is selected from alkyl, aryl, alkylaryl, and aralkylaryl of 8-30 carbon atoms, R₂ is —CH₂OCH₂CH═CH₂; R₃ is selected from H, CH₃, and CH₂CH₃; R₄ is H or —SO₃⁻M⁺ or —PO₃⁻M⁺, where M is selected from H or K, Na, NH₄, and alkanolamine; and x=2 to 100; y=4 to 200 and z=0 or 1 to 50.

43. A hair conditioning composition according to claim 42 wherein said reactive surfactant is selected from a monomer represented by the formula: 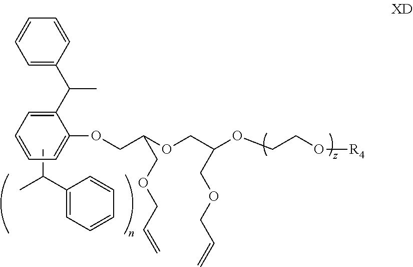
wherein n is 1 or 2; z is 4 to 40; and R₄ is SO₃⁻M⁺ or PO₃⁻M⁺, and M is selected from Na, K, and NH₄.

44. A hair conditioning composition according to claim 1 wherein said emulsion polymer is polymerized from a monomer mixture comprising at least 30 wt. % of at least one C₁-C₄ hydroxyalkyl (meth)acrylate, 15 to 70 wt. of at least one C₁-C₁₂ alkyl (meth)acrylate, 0 to 40 wt. % of at least one vinyl ester of a C₁-C₁₀ carboxylic acid (based on the weight of the total monomers), and 0.01 to 1 wt. % of at least one crosslinker (based on the weight of the monomer composition).

45. A hair conditioning composition according to claim 1 wherein said emulsion polymer is polymerized from a monomer mixture comprising at least 30 wt. % of at least one C₁-C₄ hydroxyalkyl (meth)acrylate, 15 to 70 wt. of at least one C₁-C₁₂ alkyl (meth)acrylate, 1 to 10 wt. % of at least one monomer selected from an associative monomer, a semi-hydrophobic monomer, or mixtures thereof (based on the weight of the total monomers), and 0.01 to 1 wt. % of at least one crosslinker (based on the weight of the monomer composition).

46. A hair conditioning composition according to claim 44 wherein said C₁-C₄ hydroxyalkyl (meth)acrylate is hydroxyethyl methacrylate, said C₁-C₁₂ alkyl acrylate is selected from methyl methacrylate, ethyl acrylate, butyl acrylate, or mixtures thereof, said vinyl ester of a C₁-C₁₀ carboxylic acid is selected from vinyl formate, vinyl acetate, vinyl propionate, vinyl butyrate, vinyl isobutyrate, vinyl valerate, vinyl hexanoate, vinyl 2-methylhexanate, vinyl 2-ethylhexanoate, vinyl iso-octanoate, vinyl nonanoate, vinyl neodecanoate, vinyl decanoate, vinyl versatate, vinyl laurate, vinyl palmitate, vinyl stearate, or mixtures thereof; or mixtures thereof.

47. A hair conditioning composition according to claim 45 wherein said emulsion polymer is polymerized from a monomer mixture comprising hydroxyethyl methacrylate, and a monomer selected from methyl methacrylate, ethyl acrylate, butyl acrylate, an associative monomer, a semi-hydrophobic monomer, or mixtures thereof.

48. A hair conditioning composition according to claim 44 wherein said emulsion polymer is polymerized from a monomer mixture comprising hydroxyethyl methacrylate, ethyl acrylate, butyl acrylate and a monomer selected from vinyl acetate, an associative monomer, a semi-hydrophobic monomer or mixtures thereof

49. A hair conditioning composition according to claim 1 wherein said emulsion polymer is polymerized from a monomer mixture comprising hydroxyethyl methacrylate, ethyl acrylate, butyl acrylate and a monomer selected from an associative and/or a semi-hydrophobic monomer.

50. A hair conditioning composition according to claim 48 wherein said emulsion polymer is polymerized from a monomer mixture comprising hydroxyethyl methacrylate, ethyl acrylate, butyl acrylate and vinyl acetate.

51. A hair conditioning composition according to claim 49 wherein said associative monomer comprises (i) an ethylenically unsaturated end group portion; (ii) a polyoxyalkylene mid-section portion, and (iii) a hydrophobic end group portion containing 8 to 30 carbon atoms.

52. A hair conditioning composition according to claim 51 wherein said associative monomer is represented by formulas VII and/or VIIA: 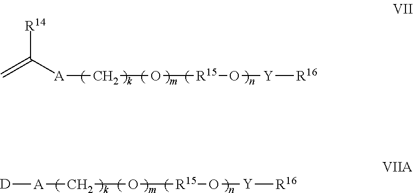
wherein R¹⁴ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; D represents a vinyl or an allyl moiety; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or C₄H₈, and combinations thereof; and n is an integer in the range of about 2 to about 150; Y is —R¹⁵O—, —R¹⁵NH—, —C(O)—, —C(O)NH—, —R¹⁵NHC(O)NH—, or —C(O)NHC(O)—; R¹⁶ is a substituted or unsubstituted alkyl selected from a C₈-C₃₀ linear alkyl, a C₈-C₃₀ branched alkyl, a C₈-C₃₀ carbocyclic alkyl, a C₂-C₃₀ alkyl-substituted phenyl, an aralkyl substituted phenyl, and an aryl-substituted C₂-C₃₀ alkyl; wherein the R¹⁶ alkyl group, aryl group, phenyl group optionally comprises one or more substituents selected from the group consisting of a hydroxyl group, an alkoxyl group, benzyl group styryl group, and a halogen group.

53. A hair conditioning composition according to claim 52 wherein said associative monomer is represented by formula VIIB: 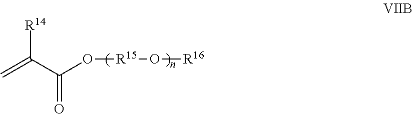
wherein R¹⁴ is hydrogen or methyl; R¹⁵ is a divalent alkylene moiety independently selected from C₂H₄, C₃H₆, and C₄H₈, and n represents an integer ranging from about 10 to about 60, (R¹⁵—O) can be arranged in a random or a block configuration; R¹⁶ is a substituted or unsubstituted alkyl selected from a C₈-C₃₀ linear alkyl, a C₈-C₃₀ branched alkyl, a C₈-C₃₀ carbocyclic alkyl, a C₂-C₃₀ alkyl-substituted phenyl, an aralkyl substituted phenyl, and an aryl-substituted C₂-C₃₀ alkyl, wherein the R¹⁶ alkyl group, aryl group, phenyl group optionally comprises one or more substituents selected from the group consisting of a hydroxyl group, an alkoxyl group, benzyl group styryl group, and a halogen group.

54. A hair conditioning composition according to claim 49 wherein said semi-hydrophobic monomer comprises (i) an ethylenically unsaturated end group portion; (ii) a polyoxyalkylene mid-section portion, and (iii) an end group portion selected from hydrogen or an alkyl group containing 1 to 4 carbon atoms.

55. A hair conditioning composition according to claim 54 wherein said semi-hydrophobic monomer is selected from at least one monomer represented by formulas VIII and IX: 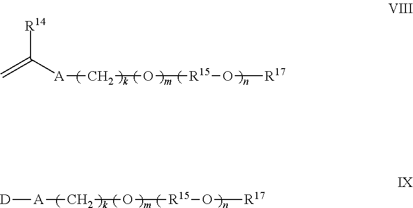
wherein R¹⁴ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or C₄H₈, and combinations thereof; and n is an integer in the range of about 2 to about 150; R¹⁷ is selected from hydrogen and a linear or branched C₁-C₄ alkyl group; and D represents a vinyl or an allyl moiety.

56. A hair conditioning composition according to claim 55 wherein said semi-hydrophobic monomer is selected from at least one monomer represented by formulas VIIIA and VIIIB:
CH₂═C(R¹⁴)C(O)O—(C₂H₄O)ₐ(C₃H₆O)_{b}—H VIIIA
CH₂═C(R¹⁴)C(O)O—(C₂H₄O)ₐ(C₃H₆O)_{b}—CH₃ VIIIB
wherein R¹⁴ is hydrogen or methyl, and “a” is an integer ranging from 0 or 2 to about 120, and “b” is an integer ranging from about 0 or 2 to about 120, subject to the proviso that “a” and “b” cannot be 0 at the same time.

57. A hair conditioning composition according to claim 56 wherein b is 0.

58. A hair conditioning composition according to claim 49 wherein said associative monomer is selected from lauryl polyethoxylated (meth)acrylate, cetyl polyethoxylated (meth)acrylate, cetearyl polyethoxylated (meth)acrylate, stearyl polyethoxylated (meth)acrylate, arachidyl polyethoxylated (meth)acrylate, behenyl polyethoxylated (meth)acrylate, cerotyl polyethoxylated (meth)acrylate, montanyl polyethoxylated (meth)acrylate, melissyl polyethoxylated (meth)acrylate, where the polyethoxylated portion of the monomer contains about 2 to about 50 ethylene oxide units, and said semi-hydrophobic monomer is selected from methoxy polyethyleneglycol (meth)acrylate or polyethyleneglycol (meth)acrylate, where the polyethoxylated portion of the monomer contains about 2 to about 50 ethylene oxide units.

59. A hair conditioning composition according to claim 49 wherein said crosslinker is selected from a monomer having an average of 3 crosslinkable unsaturated functional groups.

60. A hair conditioning composition according to claim 59 wherein said crosslinker is pentaerythritol triallyl ether.

61. A hair conditioning composition according to claim 60 wherein said pentaerythritol triallyl ether is present in an amount ranging from about 0.01 to about 0.3 (based on the weight of the monomer composition).

62. A hair conditioning composition according to claim 49 wherein said crosslinker is selected from a monomer represented by the formula: 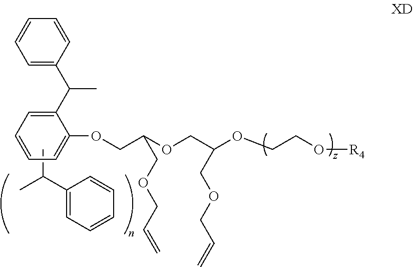
wherein n is 1 or 2; z is 4 to 40 in one aspect, 5 to 38 in another aspect; and R₄ is SO₃⁻M⁺ or PO₃⁻M⁺, and M is selected from Na, K, and NH₄.

63. A hair conditioning composition of claim 62 wherein said crosslinker is present in an amount ranging from about 0.1 to about 5 wt. % (based on the total weight of monomers).

64. A hair conditioning composition according to claim 49 wherein said monomer mixture is polymerized in the presence of a protective colloid.

65. A hair conditioning composition according to claim 64 wherein said monomer mixture is polymerized in the presence of poly(vinyl alcohol).

66. A hair conditioning composition according to claim 64 wherein said emulsion polymer is polymerized in the presence of partially hydrolyzed poly(vinyl alcohol).

67. A hair conditioning composition according to claim 66 wherein said partially hydrolyzed poly(vinyl alcohol) is hydrolyzed in the range from about 80 to 90%.

68. A hair conditioning composition according to claim 1 wherein said emulsion polymer is polymerized from a monomer mixture comprising from about 40 to 45 wt. % of hydroxyethyl acrylate, 30 to 50 wt. % of ethyl acrylate, 10 to 20 wt. % of butyl acrylate and from about 1 to about 5 wt. % of at least one associative and/or semi-hydrophobic monomer (based on the weight of the total monomers), and at least one crosslinker.

69. A hair conditioning composition comprising:
(a) cetrimonium chloride;
(b) an ethoxylated methyl glucoside esterified with a C₁₆ to C₂₂ fatty acid;
(c) a nonionic, amphiphilic, emulsion polymer; and
(d) water;
(e) an auxiliary conditioning agent selected from a silicone, a dimethicone copolyol, a dialkyl quaternary ammonium compound, an asymmetrical dialkyl quaternary ammonium compound, a cationic polymer, a hydrocarbon oil, a natural oil, an ester oil, a synthetic wax, and combinations thereof; and wherein said emulsion polymer is prepared from a polymerizable monomer mixture comprising:
i) 40 to 50 wt. % of at least one hydroxy(C₁-C₅)alkyl (meth)acrylate monomer (based on the total monomer wt.);
ii) 15 to 70 wt. % of at least two different monomers selected from a (C₁-C₅)alkyl (meth)acrylate monomer (based on the total monomer wt.);
iii) 0.5 to 5 wt. % of an associative and/or a semi-hydrophobic monomer; and
iv) 0.01 to 5 wt. % of at least one crosslinker (based on the weight of the monomer composition).

70. A hair conditioning composition according to claim 69 wherein said monomer i) is hydroxyethyl methacrylate.

71. A hair conditioning composition according to claim 69 wherein said monomers ii) are ethyl acrylate and n-butyl acrylate.

72. A hair conditioning composition according to claim 69 wherein ethyl acrylate is present in an amount ranging from about 15 to about 50 wt. % of the monomer mixture.

73. A hair conditioning composition according to claim 72 wherein butyl acrylate is present in an amount ranging from about 10 to about 20 wt. % of the monomer mixture.

74. A hair conditioning composition according to claim 73 wherein said associative monomer is selected from behenyl polyethoxylated methacrylate.

75. A hair conditioning composition according to claim 74 wherein said associative monomer contains 2 to 30 moles of ethoxylation.

76. A hair conditioning composition according to claim 69 wherein said ethoxylated methyl glucoside is PEG-120 Methyl Glucose Dioleate.

77. A hair conditioning composition according to claim 69 wherein said silicone conditioning agent is selected from polydimethylsiloxanes (dimethicones), polydiethylsiloxanes, polydimethyl siloxanes having terminal hydroxyl groups (dimethiconols), polymethylphenylsiloxanes, phenylmethylsiloxanes, amino functional polydimethylsiloxane (amodimethicone), non-ionic dimethicone copolyols, dimethicone copolyol esters, dimethicone copolyol quaternium nitrogen containing compounds, dimethicone copolyol phosphate esters, and mixtures thereof.

78. A hair conditioning composition according to claim 69 wherein said cationic polymer is selected from Polyquaternium-4, Polyquatemium-6, Polyquaternium-10, Polyquatemium-11, Polyquarternium-16, Polyquaternium-24, Polyquaternium-28, Polyquaternium-29, Polyquaternium-32, Polyquaternium-33, Polyquaternium-35, Polyquatemium-37, Polyquarternium-39, Polyquatemium-44, Polyquaternium-46, Polyquaternium-47, Polyquaternium-52, Polyquaternium-53, Polyquarternium-55, Polyquaternium-59, Polyquatemium-61, Polyquaternium-64, Polyquaternium-65, Polyquaternium-67, Polyquaternium-69, Polyquaternium-70, Polyquaternium-71, Polyquaternium-72, Polyquaternium-73, Polyquaternium-74, Polyquaternium-76, Polyquaternium-77, Polyquaternium-78, Polyquaternium-79, Polyquaternium-80, Polyquaternium-81, Polyquaternium-82, Polyquaternium-84, Polyquaternium-85, Polyquatemium-87, and PEG-2-cocomonium chloride, Starch Hydroxypropyltrimonium Chloride, Guar Hydroxypropyltrimonium Chloride, Hydroxypropyl Guar Hydroxypropyltrimonium Chloride, and Cassia Hydroxypropyltrimonium Chloride, and mixtures thereof.

79. A hair conditioning composition according to claim 69 wherein said dialkyl quaternary ammonium compound is selected from dicocodimonium chloride; dicocodimonium bromide; dimyristyldimonium chloride; dimyristyldimonium bromide; dicetyldimonium chloride; dicetyldimonium bromide; dicetylmethylbenzylmonium chloride; distearyldimonium chloride; distearyldimonium bromide; dimetyldi(hydrogenated tallow)monium chloride; hydroxypropylbisstearylmonium chloride; distearylmethylbenzylmonium chloride; dibehenyl/diarachidyldimonium chloride; dibehenyl/diarachidyldimonium bromide; dibehenyldimonium chloride; dibehenyldimonium bromide; dibehenyldimonium methosulfate; dibehenylmethylbenzylmonium chloride; dihydrogenated tallow benzylmonium chloride; dihydrogenated tallowethyl hydroxyethylmonium methosulfate; dihydrogenated tallow hydroxyethylmonium methosulfate; di-C₁₂-C₁₅ alkyldimonium chloride; di-C₁₂-C₁₈ alkyldimonium chloride; di-C₁₄-C₁₈ alkyldimonium chloride; dicocoylethyl hydroxyethylmonium methosulfate; disoyoylethyl hydroxyethylmonium methosulfate; dipalmitoylethyldimonium chloride; dihydrogenated palmoylethyl hydroxyethylmonium methosulfate; dihydrogenated tallowamidoethyl hydroxyethylmonium chloride; dihydrogenated tallowamidoethyl hydroxyethylmonium methosulfate; dihydrogenated tallowoylethyl hydroxyethylmonium methosulfate; distearoylethyl hydroxyethylmonium methosulfate; and mixtures thereof.

80. A hair conditioning composition according to claim 69 wherein said asymmetric dialkyl quaternary ammonium compound is selected from stearylethylhexyldimonium chloride, stearylethylhexyldimonium bromide; stearyl ethylhexyl dimonium methosulfate; cetearyl ethylhexyldimonium methosulfate; and mixtures thereof.
